SEQUENCE LISTING (82 sequences)

SEQ ID NO: 1 - DNA, 3885 bp, Zea maize
  source 1..3885: mol_type = unassigned DNA
atggcggacttggcgctagttggcttaaggtgggcagcatcgccgattgtcaaggagcttcttactaaagcttcagcttacctcagtgtggacatggtgcgtgagatcgaacgactacaagacactgtcctgccacagttcgagttggtgattcaagcggcccagaagagcccccataggggcaagctggaatcctggcttcggcgtctcaaagaagccttctatgatgccgaggacctgctggacgagcatgagtacaacgtccttaaggccaaggccaagagcggaaaaggtcccctgctccgagaggatgaaagctcctccactgcaaccactgtcatgaagccttttcattctgctatgaacagggcacgcaacttgctccctgggaacagaaggctaattagcaagatgaacgagctcaaagctatcctgacagaagcccaacaacttcgagatcttcttggcttgccacatggcaataccgtcgagtgcccagctgcagcacctaccagtgttcccacaaccacatcacttcccacttccaaggttttcggtcgcgacagggatcgtgatcgcatagtaaaatttcttctcggcaagacaacaactgctgaggcaagctcaactaagtactccggtttggccattgttggattgggaggaatggggaagtctaccttagcacaatatgtctataatgacaagaggattgaagaatgctttgatgtcaggatgtggatctgtatctcgcgcaaacttgatgtgcatcgtcacacaagggagattatagagtctgcaaaaaagggagagtgcccacgtgttgataatctcgatactctccagtgcaaattacgtgatatactacaagagtcacagaaattcctgcttgtcttggatgatgtttggtttgaaaaatctcatagtgagacagagtgggagttattccttgctccattagtctctaaacagtcagggagcaaagttttggttacttctcaaagtggaacacttccggccgctatttgttgtgaacaagaacatgtcattcatttggaaaacatggatgatactgagtttttggctctttttaaacaccatgctttctctggagcagaaatcaaagaccaactgttacgcacgaagctggaagacactgcagaggagattgctaaaaggcttggacaatgtcctttggcagcaaaagttctgggttctcgattgtgcaggaaaaaggatattgctgaatggaaagctgctctaaagcttggagatttaagtgatcccttcacatctctgttgtggagttacgagaagttagatccacgtctgcagaggtgcttcttgtattgcagcttgttgccaaaaggtcacagatatagacctgaagagttggttcacctttgggtggcagaaggatttgttggttcatgcaatttgagtaggagaacgttagaagaggttgggatggattacttcaatgatatggtctctgtatctttcttccaattggtttctcaaatgtattgtgattcgtactatgtcatgcatgatatccttcatgattttgcagagtcactctctagggaagactgctttagattagaagatgataatgtgacagaaataccatgcactgttcgacatctatctgttcatgttcaaagtatgcaaaagcataagcaaattatctgcaagctatatcatttacgcactattatctgcatcgatccgctaatggatggtccaagtgatatttttgatggcatgctacggaaccaaagaaaactgcgtgtattgtctctgtcattttacagcagcagcaagttgccagaatctattggtgagctgaagcacctccggtatttgaacctcgtcaggacgttagtttctgaattgcctacatcattatgtactctctaccacttacaattactttggttaaaccacatggtggagaatttgcctgacaaactatgcaatttaagaaagctacggcatctaggagcgtacgtgaatgatttcgcgattgaaaagcctatttgccaaattctgaatataggtaagttaacgtctctacaacacatttatgtcttttctgtacagaagaagcaaggctatgagttgcgacagttgaaggacttgaatgagcttggtggcagtttaaaagtgaaaaatcttgagaatgtcattggaaaggatgaagccgtagagtcgaagctatatctgaaaagtcgccttaaagagttggcatttgagtggagttccgagaatggcatggatgcaatggatattctagaaggtctaagaccgccaccccaactgagtaagctcagaatcaaaggttacagatctgatacatatcctgggtggttactagagcgatcctattttgagaatttggaaagttttgagcttagtaattgcagtttgctagaaggcctaccaccagatacagagctccttcggaattgctctaggttgcgtataaactttgttccaaatttgaaggaactatctaatcttccagcaggccttacagatttatcaattgattggtgcccactgcttatgtttatcaccaacaatgagctaggacagcatgacttgagggaaaatataataatcaaggcagacgacctggcatctaaacttacattgatgtgggaggtggattcaggaaaaaaagttaggagtatactgtcgaaagactattcatctctgaagcagttgatgacattgatgatggatgatgatatatcaaagcatcttcaaatcattgaaagtggtctggaggaaagagaagataaagtatggatgaaagaaaacatcatcaaggcatggctcttttgccatgagcagaggataagattcatttatggaaggaccatggagatgccattggttctaccgtcaggactctgtgaactttctctttcttcatgcagtattacagatgaagctttagctatttgccttggtggcctcacttcactgaggaatttaaaattgaaatataatatggcattaactacacttccatcagaaaaggtgtttgagcatttgacgaagcttgacaggttggttgtaataggttgtttgtgtctcaaatcactggggggcttacgtgctgctccatctctttcctgttttaactgttgggattgtccttctttagagctagcacggggagcagaactaatgccgttgaaccttgctagcaatctcagcatccttggctgcattcttgcagctgattcgttcattaatggcttgccacatctgaaacatctttccattgatgtctgcagatgctccccatccttatcgattggccacctgacctcccttgaatcattatgtctaaatggtctccctgatctttgctttgttgaaggcttgtcttccctgcaccttaagcgcctaagtttagtagatgttgcaaacctcactgccaagtgcatctcacagtttcgtgtccaggaatcgctcagggttagtagctctgtattgctcaaccacatgctaatggctgaagggtttacagccccaccaaatcttactcttttagattgcaaggagccgtcagtttcatttgaagaacctgcaaatctctcatccgtcaagcacctgcacttttcatgttgcgaaacagagtccctgcctagaaatctaaaatctgtctcaagtctggagagtctttctatagaacaatgccccaacatagcatctttaccagatctgccgtcctccctccagcgcataactatattgaattgccccgtcttgatgaagaattgccaagaacctgatggagaaagctggccaaagatttcgcacgttcgttggaagagctttccaccaaaatcgatctggcttccttag

SEQ ID NO: 2 - DNA, 1914 bp, Zea maize
  source 1..1914: mol_type = unassigned DNA
atggcggagaggttgcctgacaaactttgcaatttaagtaagctacgacatctaagagtgaacaacaatcaaattcccaacattggcaagcttacttcattacaacgaatagagatattttccgtgcaaaagaagcaaggatatgagttgcaacaactgaagtacttgaatgagcttggtggcagtttaagcgtacaaaatcttgagaatgtcatcggaaaggatgaagccttagagtcgaagctatatctgaaaagtcgccttaaagagttgacacttgtgtggagttctgataatggcatggatgcaatggatattctgcatttggatattctagaaggtctgagaccgccaccccaattgagtaaactcacaatcgaaggctacaaatctagcacatatcctgggtggttacttgagcgatcctattttgagaatttagaaagttttgagctcaataattgcagtttgctagcagtcctaccaccagatacagagctccttcggcattgctctaggctgcatataaaaaatgttccaaaattgaaggaactaccgtatcttccagcaggccttacagagttgtcaatttgtaggtgtccattacttatgtttatcaccaacaaggagctaggacaacatgacttgagggaaaatataatgaaggcagacgacctgtcgtctaaacttgcatcgatgtgggaggtggattcaagatcacatgttacgagcgtactgtcggaagactattcatttctgaagcagttgatgacattgatgatggatgatgatatatcaaagcatcttcaaattattgaaagtggtctagaggaaggaggagatgaagtatggatgaaagaaaacatcatcaaggcatggctcttttgccatgagcagaggataagattcatttatggaaggaccatggagatgtcactggttctaccgtcaggactctataaactttctctttcttcatgcattattacagatgaagctttagctatttgcctcagtggcctcacttcactgagaactttggaattgaaatataatatgacattaactacacttccgtcagaagaggcgtttcaacaaatgacaaagctgaagtgcttcgctataagtggttgttggtgtctcaaatcactggggggattacatgctgctccatctctttcggctcttgattgttgggattgtccttctttagagctagcacgtggagccgaactaatgccattgaaccttgctagctaccttgacatccagggctgcattcttgcagccgattcattcactaattatgtgccagacctgaaacaacttaccattattaactgcagatgctccccatccttatcgattggccacctgacctcccttgaatcattacaactaattggtctccctgatctttactttgttgaaggcttgtcttccctgcaccttaagcgcctaaagttaggagatgttgcaaaccttactgccaagtgcttctcacagtttcgtgtcatggaatcgctcaccgttagtagctccgtattgctcaaccagatgctcatggctgaagggtttatggtcccaccaaatcttgaattcttatattgcaaggagccgtcgattttatttgaagagcctgcaaatctctcatctgtcaagtgcctgaatttttcgttatgtgaaaccgagtccctgccaagaaatctaaaatctctatcaagtctggagagtctcgagatagggttttgccccaacatagcatctttaccagatctgccgtcctccctcgagcgcataactatatgggagtgccctgtcttgaagaagaactgccaagaacctgatggagaaagctggccaaagatttcacgcatccgtcggaaggacataggcttccaatattttatttag

SEQ ID NO: 3 - DNA, 24 bp, Zea maize
  source 1..24: mol_type = unassigned DNA
gcatcttcaaattattgaaagtgg

SEQ ID NO: 4 - DNA, 24 bp, Zea maize
  source 1..24: mol_type = unassigned DNA
gcatcttcaaatcattgaaagtgg

SEQ ID NO: 5 - DNA, 23 bp, Zea maize
  source 1..23: mol_type = unassigned DNA
gatctgatacatatcctgggtgg

SEQ ID NO: 6 - DNA, 23 bp, Zea maize
  source 1..23: mol_type = unassigned DNA
aatctagcacatatcctgggtgg

SEQ ID NO: 7 - DNA, 23 bp, Zea maize
  source 1..23: mol_type = unassigned DNA
ccttctttagagctagcacgtgg

SEQ ID NO: 8 - DNA, 23 bp, Zea maize
  source 1..23: mol_type = unassigned DNA
ggctcttttgccatgagcagagg

SEQ ID NO: 9 - DNA, 23 bp, Zea maize
  source 1..23: mol_type = unassigned DNA
gctttagctatttgccttggtgg

SEQ ID NO: 10 - DNA, 3399 bp, Glycine max
  source 1..3399: mol_type = unassigned DNA
ctcaataatcacttttatcatttcaaacaaagaaagttgttagtgcttaacattcctttgaatattaaactaagttcaatttttatttcttaccattagcgtgacatgaaaaaataaatttattaattccaatatatattggtggtataaagcacttccatatatgattggtcacaatttttttttacttagattccatcttcattgaaaaaagttaaaacacatgaaaaattaattatttcagttaaattaattcctttcactttttaaatatattctttatttgggtaaaataattcctttttgttatgtgtgagtttgaaattcttttaatttaatatatttgtaaataaattattttgacctaatatattgtaaatcaattcaaaaatagaatttactgtctaaagactcattattcaaccaaacgagagagttgtgttattctatagtgacggagcaacagaaaggttaagaatgtagcataaccaaaacaatgaaacaggtgatgtagaagatcatgctgtgaacggaggtagcaatggcgatgatgctttgactataaataagttgctatccgatctagcaaggcaagaagactactttgataaaagcttacagtggcgggagttccagggcaagaagcgcaagagacaagatgacaattggttggataaactaaaggacctgaaaaaaagagccattgatgtgaaaaactcactgcatcagtctgggtcgactaatgaattccccaagccttctgaattgggtgatgagtttttttatttatcgatagagaagcgttgggcgtcgcgagataaaaaagtggagaacatgtgggatcttctggaggatgaggaagtcttcattattggcatagatggaatggggggagttggaaaaacattcatggcaactcatatcaagaatgagattaaaagaaaggggactttcaaggatgtcttctgggtcactgtttcccatgatttcaccaatttcaaattgcaacatgacattgcagaaacaatacaggttaagctttacggagatgagatgactagagcaacaattttgacgtcagagttggagaaaagagaaaaagcactgcttattttggatgatgtttgggaatatattgatctgcaaaaggtggggattcctcttaaagtgaatggcattaaattgattatcacaactcgtttgaaacatgtgtgtctacagatggattgccaaccatataatataataacaatattcccctttgaagaagaagaagaagaagaagaagaagaagaagaagaagaaaaagaagaagaagaagaagcttgggagttatttttgctaaaacttggacaccgtggaacacctgcaagacttccccctcatgtactagagattgcaagatctgttgtaatgaaatgtgatggtttaccacttggaatcagtgtgatggctcgaaccatgaaagggaaaaatgagatccattggtggagacatgcattgaataaacttgacagattggaaatgggagaagaggtcttaagcgtactaaaacgtagctatgacaatttaattgaaaaggacatccaaaaatgtttcttacagtctgcactgtttcctaatgctgatgaagggaaatgggctatgatgattgttgagagtgggttgttaaatggaaaggggagtttggaggaaatatttgatgaggcacgtgtcatagtggataaactcataaaccattctttgttgttaggttattggagtttacgaatgaatggtctgttgaggaagatggcgtgcaatatcttgaacgagaatcacacttacatgataaaatgtcatgaaaatttgagaaagatacctcagatgcgggaatggacagctgatctggaggcagtttctttggcgggtaatgagatagaagaaatagcagagggcacatcacctaattgtccacgcttgtccaccttcatcttatctcgtaattccatcagtcatattcccaagtgttttttcagacacatgaacgctctaacacaacttgatttatcatataatcgtagattaacatctttgccaaagtcgctgtctaagttgaggtctcttacttctttagtgctccgccaatgttcaaaattgaaagatatacctccactgggagatctacaagcattgtcaagattggacatttccggttgtaattcgctcctcagggtaccggaaggcttgcaaaatctaaaaaagttgcaatggcttagtctttcccgcaagctgaatttatcattagtaccgttatgcgtactgcccggtttgagcaatatgcaatatctggatctccgcggttggtctggtataaaagtagaagatgtaaaagggatgactatgcttgaatgttttgcagtaagctttctcgatcaggattactacaaccgttctgtgcaagaaattcaggacactggttatggacctcaaacctattttatctattttggaaaatttaaggattacacacttggattttctgagaatcttatttatggtgaattcaagcatcgaagggtatgttttggagattgcgatggattacactatttactgccaagagaccttgcagaattacttgtaagtggcaatgatcaatgggaatgcttatgtgctcctctgtcatctaatggttctctatctttaaagcacattaccattcgggactgcacaaaattgaagagtttattctgtgtatcttgtcccttatgcactaatatccaaaacctcaaatctttgacacttaataatttggatagtttaagtgtcatctgcaaggaagatgttgctggtttaacacaatctttatctcggaggggggtgttttctcatctcaaggaattgagtatcagtggatgccatcaaatagagaagttgctgacgccagggttagtgccacaacttcaaaacctgaagtctatatcggtatcgaactgccaatcaataaaggagatatttgcaggagatagttcccacaacattgcacttcccaatttaaccaacttgcaactatactggttaccagacttaaagacagtgtgcaaaggaattttactcattaactctgaggatatattgcacatcaacaattgtcccaattatgaaaaacccagaattagtcgtgtatgattgattctgttgattcagattatggtacttattttatgtggcttagtctttgtattcgtaattcttgcaaagaaaaaacaaaatcataatttgctttttgaaatagtatactggcttttattggtaccattcataatcggttcggggaagaatgtatttccgggcattgcaatttcatatggtggagggaggtttggcaagtatccaacaactccctctctggtctcc

SEQ ID NO: 11 - DNA, 3800 bp, Glycine max
  source 1..3800: mol_type = unassigned DNA
caaagtttgatttttattttaaaatcctcaattttatagtttgtgtgtgagagagaaagatttgccctatcaaacttttcttaattgttagcattattataagtaccctatcatctttaagatattaagttagtattaatagtattagtatgttactatttgacatattttttcttgattttggttaaagtttgaagatggtgactatgttttgttttgtctatatcttttgttgcattgaagcggctgaaggagacagtaaaatgattgttggtggttcagttaaccctcaaaacaatacacaattctcagctgctttattaggaggtacgcattctagttgccattccaatatattcttaaatcttaccctcaccaattccattatactattttccttcttcacacggatgagccttttatttttttatttaggtgatacaacacatagattttttttttaattctcaataatcagttttatcatttcacacaaaaaagttgttagtgcttaacattcctttgaatattaaactaagttcaatttttatttcttaccgttagcgtgacatgaaaaagtaaatttattacattccaatatatattggtgctataaagcactttcatatatatatatatatgaatggttacatttttttttttacttagattccaccttcattgaaaaaagttaaaacacatgaaaaattaattatttcagttaaattaattcctttcagtttttaaatatattctttatttgggtaaaataattctttttgttatgtgtgagtttgaaattcttttaatttaatatatttctaaataaattattttgacatataatatattgtaaatcaatttaaaaatagaattttctgtctaaagactcattattaaaacacacgagagagttgtgttattctacagtgaaggagcaacagaaaaaatttaagaatgtagcataaccaaaacaatgaaacaggtgatgtagaagatcatgctgtgaacggaggtagcaatggcgatgatgctttgactataaataatttgctatccgatctagcaagggaagaagactactttgataaagaattacagtggcgggagttccaggccattaagcgcaagagacaagatgacgattggtcggataaactaaaggacctgaaaaaaagagttattgatgtgaaaaactcactgcatcagtctgggtcgactaatgaattgcccaagccttctgaattgcatgctaagtttatttatttattgatagagaagctttgggagttgcgagatgaaaatgtgaagaagatgtgggatcttctggaggatgaggaagtcttcattattggcatagatggaatggggggagttggaaaaacattcatggcaactcatttcaagaatgagattaaaagaaaggggactttcaaggatgtcttctgggtcactgtttcccatgatttcaccattttcaaattgcaacatcacattgcagaaacaatgcaggttaagctttacggagatgagatgaccagagcaacaattttgacgtcagagttggagaaaagagaaaaaacactgcttattttggatgatgtttgggaatatattgatctgcaaaaggtggggattcctcttaaagtgaatggcattaaattgattatcacaactcgtttgaaacatgtgtggctacagatggattgcctaccaaataatacaataacaatattcccctttgatgaactagaagaagaagcttgggagttatttttgctaaaacttggacaccgtggaacacctgcaagacttccccctcatgtactagagattgcaagatctgttgtaatgaaatgtgatggtttaccacttggaatcagtgcgatggctcgaaccatgaaagggaaaaatgagatccattggtggagacatgcattgaataaacttgacagattggaaatgggagaagaggtcttaagcgtactaaaacgtagctatgacaatttaattgaaaaggacatccaaaaatgtttcttacagtctgcactgtttcctaatcatatttttaaagaggaatgggttatgatgcttgttgagagtgggttgttagatggaaagaggagtttggaggaaacatttgatgagggacgtgtcataatggataaactcattaaccattctttgttgttaggttgtttgatgttacgaatgaatggtctggtgaggaagatggcgtgccatatcttgaacgacaatcacacttacttgataaaatgtaatgaaaaattgagaaagatgcctcagatgcgggaatggacagctgatctggaggcagtttctttggcgggtaatgagatagaagaaatagcagagggcacatcacctaattgtccacgcttgtccaccttcatcttatctcgtaattccatcagtcatattcccaagtgttttttcagacgcatgaacgctctaacacaacttgatttatcatttaatcttagattaacatctttgccaaagtcgctgtctaagttgaggtctcttacttctttagtgctccgtcaatgttcaaaattgaaagatatacctccactgggagatctacaagcattgtcaagattggacatttcaggttgtgattcgctcctcagggtaccggaaggcttgcaaaatctaaaaaagttgcaatgccttaatctttcccgcgatttgtatttatcattgttacccggatgcgcactgcccggtttgagcaatatgcaatatctggatctccggggttcgtcaggtataaaagtagaagatgtaaaagggatgactatgcttgaatgttttgcagtaagctttctcgatcaggattactacaaccgttatgtgcaagaaattcaggacactggttatggacctcaaatctattttatctattttggaaaatttgatgattacacacttggatttcctgagaatcctatttatctttgtcttgaattcaagcgtcgaagggtatgttttggagattgcgatgaattaccctatttactgccaagagaccttacggaattacttgtaagtggcaatgatcaatgggaatgcttatgtgctcctctgtcatctaatggtcctctatctttaaaggacattaacattaaacactgtacaaaattgaagagtttattctgtgtatcttgttccttatgcactaatatccaaaacctcaaatctttgaaacttgataatttgggtagtttaagtgtcctctgcaaggaagatgttgctggtttaacacaatctttatctcggagtggggtgttttctcatctcaaggaattgagtatcgagaaatgccatcagatagagaagttgctgacgccagggttagtgccacaacttcaaaacctggcgtctatatcggtagaggactgcgaatcaataaaggagatatttgcgggagatagttccgacaacattgcacttcccaatttaaccaaattgcaactacgctatttaccagaattacagacagtgtgcaaaggaattttactctgtaactctgagtatatattctacatcaaggattgtcccaattatgaaacacccagaattggtggtcgtgtatgattgattctgttgattcagattatggtacttattttatgtggcttactctttgtattcgtaattcttgtaaagaaaaaacaaaatcataatttgctttttgaaatagtatactcgcttttattggtaccattcatagtcggttcggggaagaatgtatttccgggcattgcaatttcata

SEQ ID NO: 12 - DNA, 23 bp, Glycine max
  source 1..23: mol_type = unassigned DNA
gtgggatcttctggaggatgagg

SEQ ID NO: 13 - DNA, 23 bp, Glycine max
  source 1..23: mol_type = unassigned DNA
gtgggttgttaaatggaaagggg

SEQ ID NO: 14 - DNA, 23 bp, Glycine max
  source 1..23: mol_type = unassigned DNA
gtgggttgttagatggaaagagg

SEQ ID NO: 15 - DNA, 23 bp, Glycine max
  source 1..23: mol_type = unassigned DNA
ggaatggacagctgatctggagg

SEQ ID NO: 16 - DNA, 23 bp, Glycine max
  source 1..23: mol_type = unassigned DNA
gcttgtagatctcccagtggagg

SEQ ID NO: 17 - DNA, 23 bp, Glycine max
  source 1..23: mol_type = unassigned DNA
aaatagataaaataggtttgagg

SEQ ID NO: 18 - DNA, 23 bp, Glycine max
  source 1..23: mol_type = unassigned DNA
aaatagataaaatagatttgagg

SEQ ID NO: 19 - DNA, 3135 bp, Glycine max
  source 1..3135: mol_type = unassigned DNA
atgggtggggttggaaaaactactttggcccaattggtgtacaatgatgagaatttgaaacagatatttgattttgattttaaggcatgggtttgtgtttctcaagaatttgatgttctcaaggtcacaaaaactataatagaggcggtgactggaaaggcttgtaaattgagtgatctgaatctacttcatcttgaattgatggacaagctgaaagataaaaaattcttaattgttttggatgatgtttggacagaggattatgttgattggcgtcttcttaagaaaccatttaaccgtgggattattaggagaagtaaaattcttctaacaacccgcagtgaaaaaacagcatctgtagtccaaactgttcacacctatcatctaaaccaattgtcgaatgaagattgttggtcagtgtttgcgaaccatgcatgtctttccacggaatctaacgagaacacagcaacactagaaaaaattggaaaggagattgttaaaaagtgcaacggactgcctttagcagcagagtcgcttggaggcatgttgagaagaaagcatgacattggggattggaataatattctcaatagtgacatttgggaactttctgaaagtgagtgtaaagttattccagcactgagacttagttatcattatctccctccacatttaaaacgatgctttgtttattgttcgttgtatccacaagattacgaatttgaaaaaaatgaattaatcttgttgtggatggctgaagatcttttgaagaaaccaaggaaaggtaggactttagaagaggttggtcatgagtattttgatgatttggtttcgagatcatttttccaacgttcaaacacaagtagaagtagttggccttatggcaaatgttttgtgatgcatgacctcatgcatgatctagccacatcactcggtggagatttttactttagatcagaagaacttgggaaagaaacaaagatcaacaccaagactcgtcatttgtcatttgccaaattcaattcttcagtcttggacaactttgatgttgttggtagagcaaaatttctgagaactttcttgtccattataaattttgaagctgctccattcaacaatgaggaggcacaatgtatcataatgtcgaagcttatgtacttgagagttttatcattttgtgacttccaaagtctggattctttgcctgactcaataggtaaattgatccatctgcgctatttagatctctctttttcaagtgtagaaacactgccaaagtcattatgtaatttatacaatctgcaaactttgaagttgtgtagttgcagaaagctgactaagttgcctagtgacatgtgcaatcttgttaacttgcgtcatcttgagatacttggaactcctataaaagagatgccgagaggaatgagtaaattaaatcatttacaacatctggatttctttgctgtgggcaagcacgaagagaatggaatcaaagaattgggagcactttcaaatcttcgtggtcaacttgaaattaggaacttggagaatgtttcccaaagtgatgaagcgttggaggcaaggatgatggataaaaaacacattaatagtttacagttggaatggtctggatgtaacaacaacagtaccaacttccaacttgaaatagatgtgctttgcaagttacagcctcactttaacattgaatcgttgtatataaaaggctataaaggaaccagatttccagattggatgggaaattcttcctactgcaatatgatgagtctaaaattgcgtgattgtgacaactgtagtatgcttccttcacttggacaactaccttctctcaaggtccttaagattgcacgattgaataggctgaagactattgatgcaggtttttacaagaacgaagattgtcgttctgggacgccctttccctcccttgaatctctggccattcatcacatgccttgttgggaggtgtggagttccttcgattcagaagcttttcctgtgcttgaaattcttgaaatacgtgactgccccaaactagagggaagtttgccgaatcaccttcctgctctgaaaacacttacaattagaaattgtgagctgcttggctcttctctcccaacggctcccgccattcaaagtttggagataagtaaaagcaataaagtagcactgcatgcgtttcctctcttgctagagactatagaagtagaaggaagcccaatggtggagtccatgatggaggccatcacaaacatccaaccaacttgtctccggtctttaacattaagggattgctcgtcagccatgtcatttccgggtggtcgtttacctgaatcactgaagagtctgtatatcgaggatcttaaaaaactggaattcccgacgcaacacaaacatgagttactggaaacactgtcaatagaaagcagttgtgattcactcacatctcttccattggttacctttccaaatctcagagatgtcacaatcggaaagtgtgaaaatatggaatatcttttggtttcaggggcagagtcatttaagagtctgtgttctttgagtatttaccaatgccccaactttgtatcattcgggagagaaggattgcctgaagagatgagtactcttctcccaaagttagaagatctttacatatccaactgcccagaaattgagtcctttccaaaacggggtatgccacctaacctgagaacagtttggattgtgaattgtgagaaactactgagcggcctagcatggccatccatgggcatgcttactcatctcaatgttgggggtcgatgtgatggcatcaagtccttccctaaagagggtttgctgcctccctcccttacgtctctgtatctatttaagttttcaaatctggagatgttggactgcacagggcttctccatctcacatccctgcaagaattaaccatgaggggatgtcctttgctggagaatatggctggagaaaggcttcctgactctctaataaaattaaccatatgggaatgtcctttgctggaaaaacgatgccgcatgaagcaccctcaaatttggcctaaaatttcccacatccctggcattaaggttgacgatagatggatttag

SEQ ID NO: 20 - DNA, 3399 bp, Glycine max
  source 1..3399: mol_type = unassigned DNA
atggctgaggcagttggtggtgcattcctctctgctttccttgatgttgtgttcgacaagctgtctacagatgaggttgttgacttcatccgtggaaagaagcttgacctcaatttgcttgaggacttgaagaccactctgagagtggttggagctgtgcttgatgatgctgagaagaaacaaatcaaactctccagtgtccaccagtggctcatcgaactcaaagatgctctctatgatgccgatgacttgctggatgaaatttctaccaaatctgcaactcgaaagaaggtatgtaaagtgctttctcgctttactgataggaaaatggccagtaagttggaaaaaatagttgacaaattagataaagttttaggaggcatgaagggtcttcctttgcaagtgatggcaggggagatgaacgagtcatggaatactcagccaacgacatcactggaagatggatatggcatgtatggtagggatacagacaaggaggccataatgaagttgttgttgtcagatgatagcagtgatggtgtactagtgtctgtgatcgctattgtaggcatgggtggggttggaaaaaccactttagcccgttctgtgttcaacaatgagaatttgaagcagatgtttgatttaaatgcatgggtttgtgtttctgatcaatttgatattgttaaggtcacaaaaactatgatagagcaaatcacgcaagagtcatgtaaattgaatgatctaaacttgcttcaacttgaattgatggacaaactgaaagttaaaaaattcttaattgtcttggatgatgtatggatcgaggattatgagaattggagtaatcttacaaaaccatttctacatggaaaaaggggaaattgttggctagtgtttgcaaaccatgcatttccccccttagaatcaagtggggaggatagaagagctttagaagaaattggaagggagattgttaaaaagtgtaatggattgcctttagcagcacggtcacttggaggtatgttgagaagaaagcatgctattagggattggaataatatacttgaaagtgacatttgggaacttcctgaaagtcagtgtaaaattattccagccctgagaattagttatcaatatctccctccacatttaaaacggtgctttgtttattgttcattataccctaaagattatgaatttcgaaagaaggacttgatcttgttgtggatggctgaagatcttttgaagcttccaaacagaggaaaggcattagaagttggttatgagtattttgatgatttagtttcgagatcattttttcaacgttcaagtaatcaaacttggggcaattattttgtaatgcatgacctcgtgcatgatctagcattataccttggtggagaattctatttcagatcagaagaacttggaaaagaaaccaagattggtataaagactcgtcatttgtcagttacagagttcagtgatccaatctcagatattgaagtttttgacagactacaatatttgagaactttattggcaattgattttaaagattcttcattcaacaaggaaaaggcaccaggtaaattgatccatttgcgttatttaaatctctctcatacaagtataaaaacactgccggagtcattgtgtaatttgtacaatctacaaactctggcgttgtctcgttgcgaaatgttgaccaggttgcctactgacatgcaaaatcttgtaaacttgtgtcatcttcatattgatcatactccgataggagagatgcctagaggaatgggaatgttaagtcatttgcagcatttggatttctttattgtgggcaagcataaagagaacggaatcaaagaactggggacactttcaaatcttcatggttcgctttctattaggaatttggagaatgtaaccagaagcaatgaagcattggaggctaggatgatggataagaagaacattaatcatttatcgttgaaatggtctaatggcaccgacttccaaactgaattagatgtactgtgcaaattaaaacctcacccaggcctggaatctctatcgatatcgggttataatggaaccatatttccagagtgggtgggaaatttttcctaccacaacatgacaagtctaagtttacgtggttgtaataactgttgtgtgcttccttcgcttgggcaactaccatctcttaagcagctctatatttcaagattgaagtcagtgaagactgttgatgcagggttttacaaaaatgaagattgtccttcttctgtgacacccttttcttcccttgaaacgctatacattggtcacatgtgttgctgggagttgtggagtatccctgagtcagacgcctttcctctactcaagtctcttacaatagaggattgccccaaactaaggggagatttgccaaatcaccttcctgctctggaaacactcaatattacaagatgccagcttcttgtctcttccctcccaagggctcccacccttaacatattagtgatatggaaaagtaacaatgtatcgttgcatgtgtttcctcttttgttggaatggatagatgtagaaggaagcccaatggtggagtccatgatcgaggccatctccagcatcgagccaacttgcctccaacgtttaagattgagggattgttcgtcagccatatcttttccgggtggtcgtctacctgcatctctgaaggatctgcatatcagcaatcttaaaaatctggaattcccgacccaacacaaacacgatttactggaatcactgtcactatacaatagttgtgattctctcacgtctcttgcattggctacctttccaaatctcaaaagtctcggaatcgataactgtgaacatatggaatctcttttggtttcaggggcagagtcatttaagatctttctccaaatatccaactgcccagaaattgagtcgtttccagaagggggtatgccacctaacctgagaacagtttcgattgagaattgtgagaaactaatgagcggcctagcatggctatccatgggcatgcttactgatctcactgtttggggtcgatgtgatggcatcaagtcattccctaaggagggtttgctgcctccctcccttacgtttctgtatctatatggattctcaaatctggagatgttggactgcacggggcttctccatctcacatccctgcaagaattaaccataagggaatgtcctttgctggagaatatggtgggagaaaggcttcctgtctctctaataaaattaaccataagcggatgtcctttgctggaaaaacaatgccgcaggaagcaccctcaaatttggcctaaaatatcccacatccgtcacattaaagttgacagtagattgatttag

SEQ ID NO: 21 - DNA, 23 bp, Glycine max
  source 1..23: mol_type = unassigned DNA
gatctagccacatcactcggtgg

SEQ ID NO: 22 - DNA, 23 bp, Glycine max
  source 1..23: mol_type = unassigned DNA
gatctagcattataccttggtgg

SEQ ID NO: 23 - DNA, 23 bp, Glycine max
  source 1..23: mol_type = unassigned DNA
ccaaagtgatgaagcgttggagg

SEQ ID NO: 24 - DNA, 23 bp, Glycine max
  source 1..23: mol_type = unassigned DNA
cagaagcaatgaagcattggagg

SEQ ID NO: 25 - DNA, 23 bp, Glycine max
  source 1..23: mol_type = unassigned DNA
ggaatatcttttggtttcagggg

SEQ ID NO: 26 - DNA, 23 bp, Glycine max
  source 1..23: mol_type = unassigned DNA
ggaatctcttttggtttcagggg

SEQ ID NO: 27 - DNA, 23 bp, Glycine max
  source 1..23: mol_type = unassigned DNA
attgagtcctttccaaaacgggg

SEQ ID NO: 28 - DNA, 23 bp, Glycine max
  source 1..23: mol_type = unassigned DNA
attgagtcgtttccagaaggggg

SEQ ID NO: 29 - DNA, 23 bp, Glycine max
  source 1..23: mol_type = unassigned DNA
tggagatgttggactgcacaggg

SEQ ID NO: 30 - DNA, 24 bp, Glycine max
  source 1..24: mol_type = unassigned DNA
ctggagatgttggactgcacgggg

SEQ ID NO: 31 - DNA, 23 bp, Glycine max
  source 1..23: mol_type = unassigned DNA
gcaaacttccctctagtttgggg

SEQ ID NO: 32 - DNA, 23 bp, Glycine max
  source 1..23: mol_type = unassigned DNA
gcaaatctccccttagtttgggg

SEQ ID NO: 33 - DNA, 21 bp, synthetic construct
  misc_feature 1..21: note = Synthetic
  source 1..21: mol_type = other DNA
atgagtattttgatgatttgg

SEQ ID NO: 34 - DNA, 24 bp, synthetic construct
  misc_feature 1..24: note = Synthetic
  source 1..24: mol_type = other DNA
ggttacgagtattttgatgattta

SEQ ID NO: 35 - DNA, 22 bp, synthetic construct
  misc_feature 1..22: note = Synthetic
  source 1..22: mol_type = other DNA
cattatgatacattgtgcctcc

SEQ ID NO: 36 - DNA, 20 bp, synthetic construct
  misc_feature 1..20: note = Synthetic
  source 1..20: mol_type = other DNA
ctacaatacctggtgccttt

SEQ ID NO: 37 - DNA, 21 bp, synthetic construct
  misc_feature 1..21: note = Synthetic
  source 1..21: mol_type = other DNA
attacaatacctggtgctttt

SEQ ID NO: 38 - DNA, 22 bp, synthetic construct
  misc_feature 1..22: note = Synthetic
  source 1..22: mol_type = other DNA
atggaatcaaagaattgggagc

SEQ ID NO: 39 - DNA, 22 bp, synthetic construct
  misc_feature 1..22: note = Synthetic
  source 1..22: mol_type = other DNA
atggaatcaaagaattgggagg

SEQ ID NO: 40 - DNA, 22 bp, synthetic construct
  misc_feature 1..22: note = Synthetic
  source 1..22: mol_type = other DNA
acggaatcaaagaactggggac

SEQ ID NO: 41 - DNA, 23 bp, synthetic construct
  misc_feature 1..23: note = Synthetic
  source 1..23: mol_type = other DNA
agtgaggctgtaacttgcaaagc

SEQ ID NO: 42 - DNA, 22 bp, synthetic construct
  misc_feature 1..22: note = Synthetic
  source 1..22: mol_type = other DNA
acwaacatccaaccaacttgtc

SEQ ID NO: 43 - DNA, 22 bp, synthetic construct
  misc_feature 1..22: note = Synthetic
  source 1..22: mol_type = other DNA
ctccagcatcgagccaacttgc

SEQ ID NO: 44 - DNA, 22 bp, synthetic construct
  misc_feature 1..22: note = Synthetic
  source 1..22: mol_type = other DNA
cttcagcattgatccaacttgc

SEQ ID NO: 45 - DNA, 22 bp, synthetic construct
  misc_feature 1..22: note = Synthetic
  source 1..22: mol_type = other DNA
ttgggagaagastactcatctc

SEQ ID NO: 46 - DNA, 21 bp, synthetic construct
  misc_feature 1..21: note = Synthetic
  source 1..21: mol_type = other DNA
tccagcaaaggacatcccctc

SEQ ID NO: 47 - DNA, 20 bp, synthetic construct
  misc_feature 1..20: note = Synthetic
  source 1..20: mol_type = other DNA
cgtcaaccttaatgccaggg

SEQ ID NO: 48 - DNA, 21 bp, synthetic construct
  misc_feature 1..21: note = Synthetic
  source 1..21: mol_type = other DNA
agatgcagagacgtaagggag

SEQ ID NO: 49 - DNA, 21 bp, synthetic construct
  misc_feature 1..21: note = Synthetic
  source 1..21: mol_type = other DNA
attagagagacaggaagcctg

SEQ ID NO: 50 - DNA, 21 bp, synthetic construct
  misc_feature 1..21: note = Synthetic
  source 1..21: mol_type = other DNA
acatcccattatggttaattg

SEQ ID NO: 51 - DNA, 21 bp, synthetic construct
  misc_feature 1..21: note = Synthetic
  source 1..21: mol_type = other DNA
tttccagcaaaggacattcca

SEQ ID NO: 52 - DNA, 21 bp, synthetic construct
  misc_feature 1..21: note = Synthetic
  source 1..21: mol_type = other DNA
tccccttatggttaattttac

SEQ ID NO: 53 - DNA, 22 bp, synthetic construct
  misc_feature 1..22: note = Synthetic
  source 1..22: mol_type = other DNA
aggaagactttctccagccatt

SEQ ID NO: 54 - DNA, 21 bp, synthetic construct
  misc_feature 1..21: note = Synthetic
  source 1..21: mol_type = other DNA
cccaaatgccagggatgtggc

SEQ ID NO: 55 - DNA, 20 bp, synthetic construct
  misc_feature 1..20: note = Synthetic
  source 1..20: mol_type = other DNA
tttgagggtgcttcatgcag

SEQ ID NO: 56 - DNA, 20 bp, synthetic construct
  misc_feature 1..20: note = Synthetic
  source 1..20: mol_type = other DNA
tgacggatgtgggatatttg

SEQ ID NO: 57 - DNA, 20 bp, synthetic construct
  misc_feature 1..20: note = Synthetic
  source 1..20: mol_type = other DNA
tttccagcaaaggacatccg

SEQ ID NO: 58 - DNA, 20 bp, synthetic construct
  misc_feature 1..20: note = Synthetic
  source 1..20: mol_type = other DNA
tctccagcaaaggacatcta

SEQ ID NO: 59 - DNA, 20 bp, synthetic construct
  misc_feature 1..20: note = Synthetic
  source 1..20: mol_type = other DNA
caaaggacatctctctatga

SEQ ID NO: 60 - DNA, 20 bp, synthetic construct
  misc_feature 1..20: note = Synthetic
  source 1..20: mol_type = other DNA
aagcctttctccagccatac

SEQ ID NO: 61 - DNA, 20 bp, synthetic construct
  misc_feature 1..20: note = Synthetic
  source 1..20: mol_type = other DNA
ggaaaggcttgtaaattgag

SEQ ID NO: 62 - DNA, 22 bp, synthetic construct
  misc_feature 1..22: note = Synthetic
  source 1..22: mol_type = other DNA
acagaggattatgttgattggc

SEQ ID NO: 63 - DNA, 20 bp, synthetic construct
  misc_feature 1..20: note = Synthetic
  source 1..20: mol_type = other DNA
ggcatgggtttgtatttcta

SEQ ID NO: 64 - DNA, 22 bp, synthetic construct
  misc_feature 1..22: note = Synthetic
  source 1..22: mol_type = other DNA
gatattctgaaggtcacaaaag

SEQ ID NO: 65 - DNA, 20 bp, synthetic construct
  misc_feature 1..20: note = Synthetic
  source 1..20: mol_type = other DNA
aatagaggcggttactggac

SEQ ID NO: 66 - DNA, 20 bp, synthetic construct
  misc_feature 1..20: note = Synthetic
  source 1..20: mol_type = other DNA
acaagctgaaagataaaaag

SEQ ID NO: 67 - DNA, 20 bp, synthetic construct
  misc_feature 1..20: note = Synthetic
  source 1..20: mol_type = other DNA
gaattgatggacaagctaag

SEQ ID NO: 68 - DNA, 31243 bp, Glycine max
  source 1..31243: mol_type = unassigned DNA
ttttctcttgaactgataatcaaatagttattgagatttttaattgagctgcatttgttaagaagtcacggctaaaagagttacctagttgtcagttatactattttcatgactaagcagcaagcacagatattgcagtgatacacaaccgagagcatattctccaaaaggcaaattgccttgatgcaatttgctagtttgtacactgatagaattgcttatttatcaatagtgttccaatgtataggtatctcatggcactgattaaggataaaacatggtgatttattcctttctaaaatcttttgtccctgcacaagttgtttatattaaacattgtttaccttacttttgctcacaagatgaaagaaggaagaagatagttgagatgggtggagctcaagaactcttaaatatgttaagcactgctaaagacgaccgtacacggaaagaagcattgcatgctcttgatgcactgtcacaatcaggtgaaatcataattttaatatttttttaaatagttattatcatgctggtggagaggtagattattgtgatcaattagtacctttgtggttctaaatagtaaaccagaaatgcccaaccacttatggaatttgttaatttatttgtataatattgagctggaaatcaattttatgagcaaagtgtgataagaagcatatgaaacttttatatgtttccagtttctgttatccttattcaatagatatgggcttgtaaagatgaaaatgaacataaattgttttgtgcattaattttgggacatattatacgtgcacaagcttatgtgtaacaatatctatacctgctactttccctgtcaacatattgatttttaagaatccagttcaagtaatatttatgaggttgaaagatatgcaacagtacaaccaaattagtagtgctagctagtactagccttttctgttctctttcgattgagataagctacttcaatgttatagatgaagctcttgcatccttgcatcatgctggggccatttcagtaattaggtctgcaccaaattcacttgaggatgcagaagttgagggattcaagttgagcttgatgaaaagatttcaagatctcagatatgatgtgccatcatgacttgaggtgcatgcctccttttgctttatgtttttggttggttggagcatgaaataacatgatatgagaaattaagctggcaaccaaagcttttgtggggaagagtacttgaaattactgtgtatcatttgaccaaatctaatggaagattatagttctattgtcattttagtttttttcacttgtcaaatgcgatttgtcgctcattgttctgtcaatcataataaaatggaaaagatttatgtgcatgtcaatttttattttttgaaatatgtgtttagaagataaaagattacaacaaactagtattgaagttgtaagtgtttagatactgtaattgtatatttggttaacactactagattaaatttaagcctcaactttcaaatgtgattgatcatataatgtcataaaatgtgtgtaattataggttgatgctttaattgttatttacatatgcctcaactctcaaccgtatgtcatcatcaggcttcttgtttaggttcagctggcgccttgctcgttctattttgttcgtattcctttgttcattcgatgtttttttaggaaaatatgttcgttgaaagaaaaaatcagtcaacagaagatacatgcccttacttttctctactccacgtctccacctaccctaacccttggagttacttttcaattgagcacgttaacaagcctaactaaacgtggttctgtggagataatatactaaaaaaatattattttttatttaatttaataagacttgtgcgcgtaactcttttcaaagtgctagctttcttttttggtgaattttcaaagtgctaggtgaatatgcgtatttggagatagaaagcttttttttttgggacacaaaagcttgtttaacatgtgataaacttaaaactaataatcattttttttaattatccctattcaatgttttagttttaaaaataccccatttgggaaaatagcccatctgtggatgtaaaaattactagagtacaagttaattagggttagttgttttttttttctttcttcttttcctacgagatcaagaggaacggagcaaaatcatgttttttcttccaccaaaataatgtaaaattttaaaacaaaatactaaaataataggttataatatatttcttttccttcattttatacttagtcttttattttttttaaaaatatcattctagttatttaactctcacatttgagttaatgttatacttttgaaacttcagtcattttttatttattttggatacgattttgagccttttttcttttactaattaaagatataaaatttgtctcattaaattaaactatgaatccaattaatgttcaaacaaaacataaatccttaacatgatgaaaattgaatgaaaaatgaatttcataagcaattggaaaactgaaaaaaaaaatctaataacaatatttgaaaatctattaacaatgagcagtaggcttctttggaacttgaaatgaagaataaaagagtcattggaaataaatctcaattaattgaaagatattttttagaaaatgtcattaaatagaataaaaataatcaaaatttgcttatatttgaatctaataaaaaaattgttaattactctttttgtctcgaatataaacaaaaaatactttgagtattagtctcaaataaaagtaaaatttaactattgttactttatttaatgagatattcctaaattatattttatttaattaaagttttattacttattatctctcttttttatttttgatatgaactttcaagaaagtgtagtttagaagaagaattttttttaataagaaaggtgtaattaaataatctaattatctaactactaactttttgaataaaccgtaagttaatttcttttatatagaaagaagggattaaattaattttaaaagagttcctcttcattttaatcattaatttttttgaaattcaataatcaatacctactcattacaataataaaatacaaaaacttcttcacgaaataaattgattccttgtgttatatatatatatatatatatatatatatatatatatatatatatatatatatatatatatatatatatatattcagtatataatttagtgtagctggacacatattagtgccccgtggccgtgtgttgtgcttttttgttgggcgaaacaacggccaaagcgacgaatcacttccttacgtgacacacctctgtctaatagacgataggccaaagtcacaaatactttttgattgagtattttttaatgccacatatcatatctgtcagcgtcacatgttcaaataaatccctagtaaaaacgccagaaaacaaatgcatgagcaatttttggactttggactagttacaatttttcaacgtcacatctttaaatgatttcgtctctatttagtagttgtttttaatgcggccatgcccactttctcgttacaaagcatgcattttattatttgaacgaaaatatttaatattgtgtattactcgttttacacaattgcttttattcttttttttttattttttaaatacagtcatctttaaaaacaaaatcttcgatcattccatttcattgttcacaaaacattatcctatcacatgcaccctatgtaatataatacacggttgtggataaaataattctgcacctgcccaacttttgtattgatatcatttttttatttcctctatattttccatatttatatattaattctatcactttcctgcaccccaataagtcatgctccaaatattattgttttggataagtatattgcacaatctattcttggcttcttcatgaccatgacacggcaatggagacgaacgataatgaaaggcgcgtaaatcatttgaaaggttgaattatgtaccaaatgctattatattagcatttcatacgccattctaacaataatgagaacgagttgccccacaattgatcaatatttgtatccttgcacggcacaaacttgtaagatatggccccaacttcgtcaccccatcaagttgatttcatttctttaatatttggtattttacatagaaatgctgccacaagacatgaattctacaataaacaacaagggatccaaaatacaaaagtaacacaatcgccacagcaaccaaatgcctctagaatctccccagccataccctctctccccaaataaaattttctatacatataaaaaaaacaatagaaggggagaaatgagagaaccagcttgtatttatgacttgctactaaaagcattatatatgttggtggaaatggcaagcacacttgtaaccacagctagtataatcattatcagtgcaataattttgtctcttctcgttgatatacctttaacatccctgtcatcagaaacaatgaaaattaagagactaatttatttatttattcaattcaattttcaacttgagctctactttatcagaagtcaataagtctatgctagattaccttaaaacaatagagccggggaaaatgaaggcaaggcacactgcggatgaggatcccaggaactgaaagaagtaccaaatatctgggattgctatagctgcaaggtaggagaatacaagcagcaccagagtgaggatcataaatcttttgttgtctgtggctagcataggcttcttagggaagagaacttcatctatgttggttctcaaagagaagttcaagagaggaaacaccagcatgatgtggagggcatagcttacacggaccaaactattgagcaaggaaccaactgctgaaccagcattctggtcaaaattgatgagaatgtctgactgggttgaatccccaaataacatgtacccaaataagcctattgcaaggtagatcacagcacaaagcaataatgctaatcgaactgctgttgtcatttgggatgccttggcaagctcaaacccaatggggtgcactgaaagtaaaatcaaccatcagcacagaaattttctaggcatacccttaaaaaaaatgagcaaatccattaggccaattaccattaaagtgaaatgtgaaggctgtgacaacaacaggaactgcagtgaacagatcaaagaatgaggtttggtagtctagccgaggaaacaatctaggagtttgtgtttttccttgcaccagagctgtgatagccaacccacaacatatgccaacaaatgccactgcaagaagagttgacactgcagagctgtacttcaaggactctaccaattccaaaacaccaagaggaagaacccatgttagttactgaaactcttcaattccaaatacacaagaaagcatgttatataacaaaatattcaattgtaacactcacctacacgtttgtacaataccaatggaagcataacaaagaccaaggtgaaaagcaaagcaaattcccgggaattccaccagtgaattccaaaccactgttgcaaaatgcccaaatgcacttcccctccattttgctttccagatagcacatctcctgttgttcaacaaaaggtaattattaaaatacatctatttttttctttcatcttctttctactacattttcttcttatatttctctttatttcctgtcatttcacttttttttttcctgtctttcttctacccattacatagaccaaaaattgaggtgtgcattgcggaaacagattccccacattcactttttttttcatgaatcagaactattagattgaatatcgaaggttatatttggaatacatattttaagagtatgtttggatacaaagttagaagtgcatttgacaacttttgggagctcctctaatggaaaaagattaatatttttagtagaaaactctcaaaaacacttctagtttgtatcgaaacaggcctaagttagattcattgagataagttatgagtaatacaatctgtatttaacagtgaattcccagcggtaggaaatcccgatccttacatttccattatcttgttaaattaattaccatacacaaacattaaagaaaacgacttactacatatattttttcaaaaaatgaacctttctatttatagtaaaaaaaataaaaaacaaatttcacttattattagcaacaatttcaccaatcaaaatgaatctgactgaaaacccggcaaaactcagaacaagcataccaaaccaaaaacatgaaaaaatctacatttttttttccttttttacgaattcagtagaaaggaaattaaaaaaaaagggaaaagttccgttacgttaccgatgatgataaggtagagaattaaacccccaacgttggtgatgatgacgcaaacttgcgcggctaatgctccacccgatccgaacgcctccctcatgacgccagcgtacgtcgtcgtttcgccggagtgcgtgaaccgcatcaggaagtccacggacagttccgccagcacggccaccacgagaatcatcgcgaaagcgggaactacgccgagaaccttcatgatcgccggaatcgacatgattccggcgccgactatgctggtggccacgttgaacaccgcgccggggacggaagccggcggcggcgttcctttggaatcccccaggagggggacgctgactccggcggccggagacatgccggaggcaaaattgtgaggatcggagaaagtgcggtggcggtgtgcggtgcctggcagcctactatctttgaattgaatggtttttgttttgttgtctctcacgaaaatttcacttcctctctctttataaatgatacaagtggatttgggaagttaagaaaacaaaaaatgaagttataataagtaagattttattttataagttttgtaggatgaaaagaaatatagttgaatgaggaaatttcattgaaaatagttagctagattttataatagagattaaacaataataaaatctgcagatacttcaacatgagtatgataataataataataaaaaattgttgttttctatttttactccaacatggactgaaattcatatgaatttttttgaatagtctatttttttttatttaatttaatattcatatcaaagttatttcatactgaaaaaaatattaaatactagcattctattattaccatttggaggaatgattgaaagagtgttaaagtgcaccttttcagtcaacagttaaaaataaggcgtttaattcaattcaatattacaaagttaagttggctgtataataataacagtggtagtaagtagtagagtgaaagaaaaatttttttggtcaaaatatttaaatcaagactagaagatatgcaaatcagagattacattggatgatacggtcgaccaataaaaaataaaagaaaaaacataaattgggatgttcaaatactaataataataactctaaacaaacattaacacgtgagttttctttcccacgttgtaatcattttgaatttttaaaatgttatgacacaaataataagttaataataattataatttaacatttgaattgataaaagtgtttagttttattgtagattaaactaatctttcttcgagtaaaaataacattaaattcctacacaacaggtttatcagtttatagagtaataacactcttattcttaatcgttttcttttctggaagaaaaaataaatcttagtcttgttatttttttgagaatgtaaaatataccttaaaaaattcccttaaagtttgtataattttttggtatgtaaatatatttataaataaaaaaatgtttgcgaaaagtaatatttacataacaaacactatttacagaacattgatgaaattatttttagatatataattattaatacgaatatatgaatatgttattaaagtaatcaatagttatgttaaaactgatctgttgactagacagtttgtcaatttattttttattcacttaattgctatttttttctaggtttgttctttcgttaaaaaaccttgcattggaggaaggccaatgctagttataaaaatataaaccatgatttgaatataaaattatttttagtcgaaaaacaatgaattatgttgcaagtatcactattgaaaaaatgccaacggagcccaagaaggtgaggcccaaactgaaagcgtgaagcggcccaagactgagtgaggaaataaataattatccagaaaatcggaaatggacaatccttcttgttacgcaattctgaatttgcgggttttggatttggacttggtcgtcaacacagtctaattaatatctttttgctccttcgcttatgaatcttcttcttcttcttcttgttcctgcaacgcactgaattcgatcaatcaatccatcttcaattgctttgtttcgatcggaggaaaatggccgatcagttatcgaagggagaggaattcgagaaaaaggctgagaagaagctcagcggttggggcttgtttggctccaagtatgaagatgccgccgatctcttcgataaagccgccaattgcttcaagctcgccaaatcatgtttttcctctttctctctactttttttaaattccatttcgtgtctcctcaaaatgctgatttagtgtcataaatcataattattattctcttctattgttgttattttattgttattacttcaatcgacgagtgtgttgagttttgaggtgtccgatttcccgattaattgaagtatagttttaatctgattttactggaaaatattttttgcctgattttttttttttggaacaattactagcatataaattagaattgtggatgaagtacgacaatcaactctgtgttgtttgtgactgcgctcactttcaatttgacgactaatctctttattttgttgaaagtgacgaactttgaaattgatgttggaatagttctgtttattgttcttgatttgatctatgtggcattttaggggacaaggctggagcgacatacctgaagttggcaagttgtcatttgaaggtaacattcatcagacttggggttttggagtgggctgaatctcttttgcatcctttagttctctattaagcctgcatgacattgttgtgttctgtttccatttagttggaaagcaagcatgaagctgcacaggcccatgtcgatgctgcacattgctacaaaaagactaatataaacggtatgcatgtgtctcagttgttaccactacatgcactacaatactttctcatttatgatttgtgctttaaatgctgctcttgcttccatgcagcaaggccaattccttttagcctcaatgtttctctgtataactttaatgtaaatcatataaaacaattgctacctttttgcatgaacaaattatataaagcaaatctctttgtttaatctttacatatgtgtaaatcaaatactgggcttcatatcgataaggtctaagtaggggttcagtcttttatttggattagtttaagtcagaaattgaagttaatttgtgcttgcataagttgcttccatctgattgctttctttttatggctgtctgtatgtcatagccttattttgatttgttatttgctgactattattagattggaactcatgatcatatccctaagcaggagcaaattattttgctgtcttgcttgtcttagtatgtcccacttgcattaggaagaactaagacaattaaagttaccttttctttctttgaatacagagtctgtatcttgcttagaccgagctgtaaatcttttctgtgacattggaagactctctatggctgctagatatttaaaggtatattatgtttatgatattgatatctcttctcctgggtatgatttttaatttattctcttgtccatatcccagattttagatattgatcctgcaataaaatgcgttgaagtatactaagttatctgaatccccattaacatgttttaactgggttcactattttatacacaggaaattgctgaattgtacgagggtgaacagaatattgagcaggctcttgtttactatgaaaaatcagctgatttttttcaaaatgaagaagtgacaacttctgcgaaccaatgcaaacaaaaagttgcccagtttgctgctcagctagaacagtaagatattgtcctttctgcatatattatctcttttattatgctgatgaattgatcaatatttcttcaacttgggtttattctttaattggttagtaatttcttctgagaactttctttctggcctttattttgttcagtaccctttctctaacccactctcctcaggttaacattagcttaggtcagtgtaggttgtttgacactgagtttttattggtatggatgtatggtctattatgatctcaatggaaatctagcatattttttttccacaatccataatatgatgacttgtgtacatggtgtgaataaaagtcagtccattgctgcatttggtattggttacgtgttactgtactttctgcatatattatctcttttatcatgtcgatgatttgattaatatttcttcaatttggatttattctttaattggttagtaatttcttctgtgaacttctagttagagcatgaactgctaaagaaatccaaaactttattttttacatggaaggaactttatcagagttttatttatttatttatttttatgttaaattgaactttaactgtttctatgttatgataactcttcttcagatatcagaagtcgattgacatttatgaagagatagctcgccaatccctcaacaataatttgctgaagtatggagttaaaggacaccttcttaatgctggcatctgccaactctgtaaagaggacgttgttgctataaccaatgcattagaacgatatcaggtctaagttttttcaatagttcacttctggagactggacagcttatttgttgctaaattattcagatatgtttttattttgcaggaactggatccaacattttcaggaacacgtgaatatagattgttggcggtaggtcactggttttgaaatttcgttatgaattttttatgaccaagtaaattggattagaatatttgaacttctttgtagctgtctcctgggtcataatgttttattatatttttgtatttatcatagcattgtgatagccctgttactactttgtttgctgatttactcatacatttgccagatgaaactgacatttttttttaatcctggtggataggacattgctgctgcaattgatgaagaagatgttgcaaagtttactgatgttgtcaaggaatttgatagtatgacccctctggtaagctccaaaagttgttaaataggataacttctagtggtgtttaacaaaaaaaaaaattccacttgtattttttatccacattttataacagaataatcataacctttcacaacttaattctcaattttcacagtaattaaatgtgtaattttaaaaaaatattttccttaacttaaacctgattgaaatttccccctgaaatttaagttctatttgattacctagagtgtaatttccgtgttttgtcacttaatcactgtgtaaagttaatttttttgcttacaaaggtgtcttgtttggaatgctaaaataacaagtacacgtgtcaccaaatttagtaggattaacatttgttgttttttgccataataaacggttgaacttaacatttgttgtacgtgtcatcaaattctacaaattgtgagctgcttagtgggttggacaaacattttagcaggtggtttcgattgcctgttgaatacgtgaaattaaaccaaggcaaaattataatttgtttcttttgtctgtgtttcactcatacacattgaatcttgatgatacacagccttgttaattgttatccttccaattttttttagtgtttttgagcatctattcttgttggtcatgtgttttcttcactcatgtacctggttcttttcctacaacgataaatatgtatcctttgttttttttttccaactaaatatgtaatttcaaatttctaatcaatcattgcttccaaaatactctctctgtttcaaaataagtattatcctatattgttttacaagaccaagaaaagctaatatatagatgaaagaaattagtaattttacaaaactaaccttagtattaatattatactgaaaaactaaattgacacttattaggggtgttagtgtaaaaaagcaattaatattacattgaaaagctaacatgatacttattttgggacaacttttttctttcaaatgcaacacttgttttggaacggagggaatactagatattgtgctcccttgtatgccctggacataacgtatttaactggtctggatgagtttatgaatgtcattaatttagggggagtcatttagaatagcttacctataagtactttctaacttttctcaattagtttcacagtgcaatttattaaaaatgtctgtatctaatcaacattgtctgtgtgcttgtgcaggattcttggaagaccacacttctcttaagggtgaaggaaaagctgaaagccaaagaacttgaggaggatgatcttacttgaattgtacctttaatattcctggtggttggacctattgtttgatgatttacattgtactactatgtgtgtctgtccagttgttcgtcttacacatccacttacagagtgtacctgctttggttttaattgaattggtctgactgaattcaaaaaatgagtttttaatgaggttgctggagtctgatcttatctacattattatgaattgaattactatctatacttgttttacaccaggtttatgcgaacctgttagcaatactaatccatgttactttatgcggataatccataaaaacagaaagaattaaccatcacaatttattgataagtatatgatagcatattttaccgagagttgaaatttgaaaataagtcgtttctgtatgatttttgcttattttgaccgacattaaatacctacctggtaaagaaggatggaacatatcaaggtagggtatttcttattacaagacaaacaaaaaatatagtaacaaactgggtgttaaaaaagtcgtgtaatgaatggacggtccgcaaccgcaaattatacctttgaactagaaaagtctatagcttcctttctttttcttttttccgttcctagactgttctactttttgtaaaggaaaagattcaaaagagagaacgcaaaataccacgcggcaattttttacttttttgcttgattatcttatcgtggaattccttttccaatctcaataactttttattttcaaatgtgtttttagattttatacttttgtcaatcaggttcacctactttattatattgataaacttggtcctcaaatttttaataacctgaaaaattattttcaattcattaagaaatcatgatttttcaaaattagaagtgactgaattcacatattttttaaagtttgagaactaaatttaacaatttaaaaatagagattcatcatgtttcacaaaaaatataaggactagaaatattttttcttcttcaacaatttactcttaacattaggctaaaatagaatatttgttcttacggtttgccactttaggaacaaaatttaacttaaaatacaaataaaggattaaaaatccattttaacttatttttaataaaatactgtatgatcattaatctaattaatatgtttttttcttgatgaaagtatgactttgttaatcataataatctaattatacgaagaatcgcgtgaccttatactcgtgcttggaaataaaagagagacatttgataaattgctttaacttttgatcttctttcttacgtagtagttttttgtcccacaaaatgaattttgcatgtattcagtgcttccttcttcggcttcactttttctggccggtggcagccggtaaccagtagtcatatttggatgttaaagagacagaaaatcttaatcaccgctcattgtttgactgcattgttacctgcgactgcgagtacgtggattcattcttgtttcgccaagtgtatgagtgcgtgtcagtgtgactcttttttgtaactttttcctttgtattagcgcttagacttttgattccaaaccatttttcttttctttgtttctgaaattttatcaatcaaacataactctgaggaaatcacacttccccggccgtcatgagtcagctaattaattggtatgtctaattttcaccaccaccttatcgggtcttcaaaatttaatgctattattctcagatttcttttcttgttcctacattctgactttgcatctgtcactgttctgagaactgaattttgttgaattattagaaaaatagaaatagattctgggttttcatttctagttttaaaattgtttattaagcatatgaacaccgtcaccactcaccatagctcaattctctgtctttatttttttatttatttgctttgatactttccccggtgcttatatggatgcatagaaatagaaaatttgctacagacttgtaaccttggcatagtttcttcatgtcttcttcgcagtatttttctgtacctatatcattgatgatacggttctgatttgatcgagaaagctcagttttttatcagaatggtttcggttgatgatgggattgtgaatcccaatgatgaaattgagaaatctaacgggagtaaagtgaatgagtttgcatctatggatatttcagcaactcaaaaatcatatctgaacagtgaagatcctcagagaaggcttcagggaaccttaataagttcttctgttactaataggataaactttcttaaatttggttctgcatctgccaaattcaaaaggcttgctactgagagagaccaggtttctatatctgtgccttctcctcgttcaaagagcctaagatcacgtttcagtggcatgtttgctcagaaacttgactgggcttcagtcaagaaaatgtgcatggaatggattagaaatccagtgaacatggccctttttgtgtggatcatttgtgtcgcggtttcgggtgctattctgttccttgtcatgacaggcatgttgaatggtgtgctaccaagaaagtctaagagaaatgcatggtttgaagtaaacaaccaaatactcaatgcagtgtttacactcatgtgtttgtaccaacaccctaagagattctaccaccttgttcttctgaccagatgaagaccaaatgacatctctagccttaggaaggtatattgcaagaatgtcacttacaagccccatgagtggacacatatgatggtagttgtcattctccttcatgttaactgttttgctcaatatgcactttgtggtctaaacttagggtataaaaggtccgagagacctgccattggagttggaatatgcatatcttttgcaattgctggtttgtacaccattcttagcccacttgggaaggactatgattgtgagatggatgaagaagcacaggttcaaattacagcttctcaagggaaagagcagctgagagagaaaccaactgagaagaaatattcatttgcatccaaagatcaacaaagggttgttgaaaatagaccaaagtggagtggaggaatacttgacatttggaacgatatttccttagcatatctctcacttttctgcaccttttgtgtgcttgggtggaatatgaagaggcttggctttggaaacatgtatgttcacattgccatttttatgctgttctgtatggctcctttctggatttttcttttggcttccgttaacatagatgatgacaatgttaggcaggctctagcagctgttggaatcattctttgttttcttggtttattgtatggtggattttggaggatccaaatgagaaagaggttcaatttaccagcctatgacttctgttttggcaaaccttcagcttctgattgcacactttggctaccctgttgctggtgctctctcgctcaagaagcgcgtaccaggaataactatgatcttgtagaagataaattctcaaggaaagaaactgatactagtgatcaaccatcaatttcacctttggctcgtgaagatgtagtgtcaaccagatctggcacaagttctcctatgggtagcactagcaactcttccccttatatgatgaaaacatctagttctccaaattcaagcaatgtcttaaagggatattacagtccagataagatgctatcaactttgaatgaagacaattgtgaaagaggtcaagatggaacaatgaaccccttatatgcacaaaaataatatcaaaatactatagacattaactctgtcttatcatcttttatgaatctcaatatatttctgtgcatatatcagcatcagcgtcactgaattccttggacattgacactgaacactcatgtatgagctggagatatttattgtctttattgaacaaattcaagaaaaagaagaagaaataagcctcgaactttgattcttaaaaatgtgtatttcattttaatatctaattctctaaaacttttactttaatttctaatttgatatgttgtcatataataaaatataatttagtttttctcatttcatttttattggtataaaaaattaaaaagaataatattttataataccttacacatcaacaaacaaagtgaagatttttatgcaataaaaaaattaagccattaatacttatactaaaatgaaggtttactcaaacaaattataaaaaaaaaataaataagtaagacattaatataatgtagatacccaacaacataaatggtcgacgtgtcacggcacacaatccaattttctttgctaaaatactataaagtataaacaaaagagtatatgattagcattaccaatgaacgttaaaaaaaaaaaaaaagcattaccaatgaagttggggccatacttcccttagtaccaaaaagaaaagcatatgaagttgggtttaacagaaaagagttagttctactgtgaaatttgtctaaatttaatatccggtcatatttaaaatagaattacctttaaaaaaaatacatgtgagaaactaaagaaaaattgtttaacattaaaaccatcgacactctataaatgatgccctatccacagaccactctttagtgtttttaataccctgactcagcaagcaatataatccacttactccccaaacctaaaaaatatacaaaagtcaaacaaaatgctatttatgtgaccattttaaatacaaggaaattggagtttcagatctttttttctttgcaaacttgccacctttgaagtgaaacataagacgaaatagatagtgacacactgacagtgacacctaaatagtagctacattgatgcattgtgtctagtgtaattccatgactcattgaggagcttttccatagatgaatagcatgtttatggtatgggactgtagagtagtggagagttgcttctgggggagtgcttatttggagcattggcaaatggcaattgccatccaaaacaatacaatacattacacatactgataaaatagtacagtggatatgatagaccatgcttatgtcttatgtgattcggattgattgcattccatattagaacagttgctattgttactatgtcatttgaatttaaatttgagaaagagttagtagtgaaattacatgtcatttaagcacaatcattatatattaaatttattaatttttataataattatatttaaaatcatataagtaatgattcataattatatgacaatctaaaatcaatacataatcatcaaattatttaaattttactgtcaatatgagaattttgccaataattaatatataaaatctcagggattttaatttattgagtttgtaatttttaaatttgaacctgaccgatgattaaaattaactcatatttttctcttccatttagtttggattgatccagtcatctggtagatctagatatgggttcactcttatgtgtattaactcgttatcattcattttttctttttgagaaaatgatatgctgaataattatacgcgaaacctcctttttcttatatggtaccatttgaaatatgagttagtcataaaagaaatttgaattataagttgataaaataagcatgctttcttttttcttaaatttttaatcattattattatgatgatggtttttgtttggaatcttagtcggtgccgtcagttgaatccgaaaagaatgaatagacagaagtgctttccgacccagagaaccaaacagtagctaactttgcgcttcttgttggtgttgttcgttgagatgcatagatggtgtgaaaattcatttttgctttttttacgaatgaatggaaacagattatctttttttaatttaaatttctttttgccttgtttcctgcaatgcatctttttttttttttttacgaagaaatggaaacagattaactttttttttataatttaaatcgttttcccttgtttcctgcaatacatctaattgcgtacaagatacaactacatttgtaattaacttttttttttattatttatatgtcactttaaaaatatagagtattatgaacggcgtctttacattttttcaattaaatgaccttggaaaattatgtacattatcaacgtataacctattttctcattattgttttatacacttgccataaaatttgataataaattgtttcatattaagcaaaataggctataattgaagtaattttctaactcatttttaatattaagcaaaatttaagtgaaatttattagatcactaaatcctattatttattacatattggtttacgtgagttatcgaaaaaaaaaaggttacgtaattttgcaatattcttttcccacattaattagcactaagacaaaaaaaaaaaaaaacattgtttttgggagtgaaaactgaaaagtctaaattgccaaaacttgtaatacctactatggttctacctagcttaccttgtggattgtagttaagctaaacccgttaaagacttaatgcaactaagtaaccaaaaaacaaagacaagcgaacgcatttgtatatacatgctttcacttatcactttttttttataaaaaaagaaaagatagcaacaaggtttgaacatatgatcatgtataaaaaatatgttttttatttattattttaccttaaatatgaaggaaataaaaatatacttttattgaagaatatttattaatgatataactaaaaatatgtcaatcatagtattgtaatgaataataatttaattgactaaaaaattaatgatataacaaattgtatccagtaaaagaaactaaaaatgtagtatcaatcattaataaatgataaaatcaaaatttacaaaacaacatattttagaattaccaaggtatcaaaatgttactatttcctcctccgtttcaaattatatgatattttaaaaagaaaaaaattgttcaaaaatagatattattttacaaaattaatgtaatattaaatatatatttttaaattattcttaatcaatattcagtgaaaataatatatagaaagaataaattaattgttaattaaagagataaatattatattaaaaattacttaatatttatttgaaattaatcgtatttattattattaagtgtgctttcaattctttaacgtggacaaatacttatttctgcataataagaatattaaaggtgaagagaatatccgatccacatatttttggtcaaactccttcagacatcagtgaagtccttaaaaactcaaattaagtcaaaaaagtaggcttgactactatcatcaatttgaacactcatgacgttgcaacatttaaaacccgcctactatttccatcttactccacttctattccatcgcccaatcaaaacccaacacgtcatcaaatcaaaaaatattcaaactagtaccgcccgcacccgatcgcaagcttcccggtacggaattcctatataaacccctcttccttcccattttaagacacaactccaacaaggcattgtttggtttggtttggttacttgaaaatctcgaatcgcttaattttgatttagttttccaccgcaacgcggaacctctttctcgaactggctaactctcaggcaagtggctcggacgctgattccagcaacacgcggctggtggttgcactgtatgacgccctaaactccggcgactccaacgccgtcgtcaagatcgtcgccgccgatctcgagtggtggttccatggtccgccctctcaccagtttttgatgcgcatgctcaccggcgactccgccgccgacaactccttccgattcgttccgcagtccatcgccgccttcggctccaccgtcatcgtcgagggctgcgactccgcccgcaacattgcctgggtccacgcctggaccgtcactgatgggatgatcactcaaatcagagagtacttcaacaccgccctcaccgtcactcgcatccacgattccggcgagattgttccggccagatccggcgccggccgtttgccctgcgtctgggagagcagcgtctccggtcgggtcgggaaatccgtccccggtttggttctcgcaatataaaatataaaataagtaattagggaaggacgaggtcacgtgttgccgttgctataataattaaataagggacttgtgcacgtggcggtgactggatcgatcggtttcagggaacattgatactttgtgttagtattggaggttagggagatgtgagagctttgttgttattggtgtggtttgttttgtttgcttgtgtgtttttcaccactatgggcgtattcaggtggttgtatctttcttttgttatttggagtgttgatgatgatgcaataagaatatctatggactatgcttttaagagttgggttgtgatgatgccaaatattctatttcgtctttgtcttctcattgttccaagttaattaattgggtaatgtgatttttaaatctaactgctcttggacacccagatgatccaagtcatctccttctcttctatatactaaaaaaacatttgctttctctaaaatgaggatttgtattttagagaacaaaaaaactaaatttgatgacaatttcaattattaatttcgtaattgatagttattattatattttatcctctaaaatgtcatttcctcattttacatgaaccaaatccagaaaaataaggattttttacttgcattaaatccaaatatggatataagattattacataactttttggtactaattcctcgcggatcatgtgtcattttagttagcataattccgacatttggatataaggatttattttattttattttctatcttcaataatttgtataaagtttgagagagaccattttgcacatatgtctttctctttttctctttttctttaatttaagggaaaataatcgatccaaacaattaaattaaataataagaattatataattttggtaattacaataattctaagtcacgaacagaaaatgttagactcatttttttaactttttggaagtggtggggctaagatccttcaggaaaattccaaaagggatgcaactgaaagagggattagcaaaattcctcttcgataatttaagaaagtgaaagataaatttggttctactatatgatgtgatgttgcgctttacatatgctcgttgcttggtgcatacttgattgaaaagactcaattttagggcataaacaatacaatacccttttacattacatcaaaacttctaataagttgtttcaattcgtgcgtgtattgtctatttatcaaattgccaacaaccctaaattatgatttaattggtctaagagattatgtcaatggtgtaatcggaattgaggtagagatgcgcctggtatattgtctaaccaataaaccaattgaatttggcaacttgaatcaaatttcaaatatcatctttgtaagctcagctactaaaaataagtagaatgcatctgtttttgttttgtcctctaatattggtctgaaatatctattaattttgttattattaattaatttttgttaaaaaatttaattgattatttttgttgaaatatttcttattaattatatttttttatttacagaacttaaatctcttgtttaaaaagaagattaagatccaacaccttgtttaaagagattaaaaaatttgatatcactaccaattgactttttggtaaatatgtaggatgtataataatgataattagagtttttatgaaaaatcttttaggggcaaaaaattatttttaagaaatatatatacgacttttctagaatacgtggaggcacagctaacaggaagctggagtaaggtccaccatgtcttgggctaatacaacataaacctcttcacttccttttaaattgcaataatcacatgcatcgcatcgtaacgcatggtcttgttttttgttagatgattggtcactttcccacggtcgcggtagaatcagccaccaccatttcttttttcttctaggacccatttgtcagaccaggattcgtggactcaagatattcactgtactatgtgcattcattttgctacatgtttaggaacaaaagagaagagaatctctatgttaagtgctaattacacataaccagccagataggggcatccattgatatctttgtttttccctctcatattgtcacgtttttatatattcttttacatcttaggaaggaactataaattaaaaacacccatgtgcgtgtcccttttaaacttaataatttaggtggatgcatgatctggaactcaattaagtatagcttaatttatagttttattttggagcgtgctaaataaaataattaaactccatgcagagcaggattttattccttcgggataattgtgtttactgattagtgtgcatgaacaaaagctgcggctctttgttttatacatttgtttgatactttgactagataaaagattaaaaaaaagttacagcagaaaataataaatgtatctgtgaatggaattataattatattgtttcaaaaaagaagcaaagggtcaaattttcctatacaacacaacaatttaacatctatctccttgcatcagacaccaaaccacaaatccggcactggttattcattgatcaattcgaactcaaacaagaagacacaaataattcaccgtctcttatggatgacttcgcaaatccaagacgtaacttatttattggaatttgagttcagacagaaagacacaaacaaatggattacaagcctcattattccgtcaggaaaaaaaataggtggttttgtttttctcttttcactatgtctagtagagtccacaccacatactcactagtacaaagataacaagataaacagttgaacacccgtactatttcaacgaaaaaaataataaacgatccaaatttaacaactaatattgtggagttgttaaatcatagctataggtcaatttgcacaaggaatgagagatccattagaaccaattagtgttttccactagtttatgacatctagttctcatagaactttaaactagttgtaactaatttcacaaatctaaagataaagttattaaagcttaaattttagacttattaataaaaaaaataccacatatattgataaaaaaaaaagcccaacatatacatctttcattttaagtagaggagtgcttcccccaaaaaccctagaaacaataacaccctaggaatcctagtaacagaacacgacaacacgtaccctttaattttaaggggacttctccctcctccttggagagcttctacctccattggaagacttctctcccatctcaggaggttttcttttccaacatcacgctccccctcaatccaccacctcttttcctcatctttcttttttcctttttcagttgctattctcatctcttctctagctttataatttttttgtttgtttttttaacattcccattattttttattcctatcacatttctcctttacgatggagcttgttagaggcctatatattgcagatggatctaacagaatgaaagatcgatagaaaatattagaaaaaatgtcattaacattgaaaaaaaaacatttcttaaaaattatttttaagtctcacttttgctagctaacctcgagatcacaaattatatataactttttttttatcgtgctctgttgcaatttcatctcaaatttattttgataattaatttaatttttaattatgatttttatttgtctatgaatattgatatatcaatacgactttaaacattaaactaattgctcaaatagtttacacaagtactaatgaggtcaaggttgaacaactaaccttgatcgaattctagattgctctaatggttcgtttagaagggatgaaaatgcattttctatggattaagatttgggatgtttagatgacggtgctgctttcaacacacctgaaacattgtgaatatatttttaaaaaaatctcattatatagttaataaaaaatattaacgtcatcatttaaataaacatttaataaaagaatatgaagaatttgacataaatattttcacttggtgcatgtttggtaacatgtgagttgaactcgatacgattttttaaaatggtttttgtatttatttatttttaaaatatattttttaccaccaattcaaaaacactatactatccaacaacacgtgcattaactaacatgggatttgttaagtttgatatgtgatcttgagtttttgagacctcactatttagttatattaaatatttgaaagaggaaaaataatttgcaatccataataatcttacaaaagtgaagtaaaattatttttttataaaagatacatgtgacaatgttccaaaacacactattataattattattatctaactcatatgcattgacaatttgattcagtggagaggagagagaagttatcacgttaagtggggattagttaattaagtgcacaaatacgtcaccttcgttttcaatatgccattctgcttttcctcccacgttttttcaagccgttcgatttgttttctgtaattgttttcaaccaaggaagagaataaatccaataaaaggacatactgtcggggtagtgatcgtttgatgatgatattgataacattggcatcttggagaatatcgcataaacacaattttgattttctctttataggagtttccctgaactttgatcattatacgtattcacataacagaatccattttttttttaaaaaaaaaaggatccatatttcatttcctcttaattatttgcttccttttcccatgtaggtgactaggtggtgttttggccagggtcccagacttgctaaattgcacagttaaggaatattttcatcattggattccactaacaaaccctgaagagtcttatcaatatgaattatttactatgccaccatatatgatatatgttaaacactaattagtgatctgatgctaattgtgaagtgctgagaaattttaagctacgaaagcgatcgtatgaaagataagtggttttcagcataaaaattaaaacatattataaaaaaataataacacatgaatcattccttaatttaaatattttatcgacaattcttatttttttatttttttctcacatcataaatttcattatatttatattttttttctctctttctaaacgtaggaattaagataatatatgctatgtatatcaaacactttcgattataaagaaaaatatttgagatatttagaaaagtttgtacaatgatttagaatgggagaactacttcaccgaattctgtggtaaagaagggtgacctcagttttttctttgcttatttcttcagttaattattactactgttattattgcatgtacacccaataagtacttaaattaaagaggcaaaaaataaagaaaatctaattttattaatgtaatatttttatttttaatttttagaaggttacaggacacatcagttaaaagagtggatgtcatgatttttagaacaaaaaaagagaaaagaggaaaaccttaaataacattgaaaacactcattaaaaaagacttgagactttgaaaataattcttaaaccttaatttttaactgtgttaaatgatgtcatgtgatttatataattgatttcacttaataataagacttttgctgttattgttgtttatttattttgtttattttaacttgcacagttggtgcgtagaataaaaaagaaattaatcgttataactatagctatcaatacaagtagatcaagctacaagaagtttttatcatagaattgaattagttactagttgggtgtgtttggttcacgcattaataacatagttttcatatctggatatatggaagtgatctcttatagctttttgcatcttttattcaatgaatatgatcaatcactgcacaatgcatttccgaacacttggttagtggttggaggaatctagtctccgaaattgtccatgactttaatttaaattaggtgttacgttgttatcatttatcaagcacccgttcccgtcacttctggttaaaatccattagcctttgctgctaaaaaaacttagaatttatcttagtggggaacattttgatacaacctaaagtaagcattcaagttttgaagacagtaagcaacttaaattttatggctctctttactaatgacgccattagttaaccacacgactcaaaatttaagttatttgatatttacgtttaataacataacactacctcatcccaatagccaaatatgtatatatatatatatatataaaattgagagaaattttactattttcacgtcttggtcagttccattcttgtagaaagtcatttcctttgttttttctgcggcttccacatcactgtcgacatttttccacgacctttacttttttttctctagcttatgagataagataagaggcagcctattcatatcacattatcatataccaccactgatatatgaatttttaagcctgtgttgcgtccttatcataaatttacatataaccctacgtgtgttatctatattagaaactgaaatatgttattaacattaatatacaccactgcataggataagtactactttcgactttcaaagattaagaaaaatgaggtgtccatatcaaaacagtgggataaaattatatattaagactaattcaaaatgaatttaaatcgggcttagttttcacatgcttaaatatttccatttctatatcttagacttccaattccaatgttgaattgtggaagtaaattagcatatataggaactattttaagggatgaaggcgagcatactactcctaataataaaggtatctgaatgaactcaaatgaagaataagagataacttgtaaaaaaaaactattagaattgagagaatattattttatcaataaatataaagtattttttattatttatatattttctataactaccataacagaaaactaattacttctaactcattataacaaacttagtttagttaggtatcactaactcaaattacactcggatgggtaatacctcaagacatcaattggcgtaatgtcatagtcatagtcatataattctcagatcttgagaacaatacaagcaagattctgaaatgacatcggatcggtatcttattccatggctagcctttttaattggttaaactgaagaagaaaaaaatagcacaagaaataaagagagagatggaatcagatcgcgtatatgctcattgctgtaagagaataaaattcataaacgtgctagtttttaagaatattcgtagatcagttggaatgtgacaatcgtggttgctgaaagttttggaataatggtctaaaatggccaattttatggcagagctaagatgctttagtctaccggggtgtcaagacaatctagtacttcagttggttgattaagcctgtgtaaattattataaaatttgtaatattatctttaattcttatgaaaaaaaaataagaatatattgggagtggattaacacatcattcagcttatttttttaaatgaaattagataaagatacattttttaagaaaattaggtaatttaaataatatcatctataaaagtacttgcctttttttatgtattcatgtatcttacttatttgatgaatgatgataaatgttaaattaatattttctctttgttaattcaagaaatattaaaacattgagatatataaaaatcattactattaaaatattatttttattaattaaatatctttatatttaaaattttaaacgtatgcaattcttatgttttatcatcaaaatcttttctaaagttaaatcaatcttttctgcgtatttttatttatatttaagataccttatatttaaaagtacgtattactcatgcataaaatactttctactttttttaaataaaatactttttattttttataatttttttatatctataataatagtctaaagtattcattaattttattgataactaattcttactgtagaatctgatcaatcatctttagtgtctttcaaattacatttaatttattcgtaagactaaaacatgagattttgtttaattagatcaaactcaataccacttgaataaattacttgttagtattacttttcatattatgttcgcaagctaaaaattatagcattagttgaatttcatctagccatttacaaactttattcaatgggccaatttctatgaaaaaaaatattttatcctctaaatgttgtatatattttggaatttgagagaaacaaaaaaaaagagaaagattgtgaatgtgatagatgatataataaaaatataaaaaaaaaatagaaaatgatataaaatagatgtaaattttagggtgcccaaaataattgaatctatgtgtagtaataataatggaaaaataatataaaaatattataaaaagtgcaacaatttcttaactatagtctataggttacttaaagtagggttgttaaaatgagtagatccgtttaactgagtcagctaattaaaatgtgttgattgaatttgaaaattttgaactcaatttaattttgattttactttattcactccaaagatttgtttgaacgtaaagttgtaatgagactagttcgttagtccattttcttcttaagcaaatatatatcacccattcattaaattgagctaggttttgaattaaattttacaaaatttgaaatagtcaaatttgtcaatccaacttgattagaaattctcatttaatgaatgaggggaactagcctgaattgctacttgctatatacctgaagtcttactttcattgtcatattataattatggagtgagaaacaaattattttaaaacaatcattaatttaatgttttaatgtaatattaattattttttatttatatctcttataatattaataataaacatcaaaatttataaataaattaataataatataagattaattttataaaattattattttttaatttgtataaaataacttaagattagtattattttaaaagggatggagtatgatatgcaatataaagcaataaaatcatatatatctgccatgtcagcatgtataatgacatctaaattaaaatggctggtggataagattttttgaacttcgcttcttatctctcaacaactggaggctattatgtccactttctacgacatcaaacacatgttaggtgcatttggttgcaatttgcaatctataggattaggagggtgtttggtatgtagcttacaagactatttgctttgaattttcaagtgaccgtatccagagtttttgctaatatctcatcagatattagacatttagttttcattagtatctatctccgcgcaatgcaagagacttgacgttaagaatttatgagaaccacaggtggaactgcattagcatccttcaattttcacgaaaatgatatctattactcaaactaaagtggcattttaagctggaattttaagaatcataaattttttgagctttgtttgtgtggagtaaagactcaaatgagaatacttattttatctcacttatttaagtagaaagaggtcaattaaggaaaaaaaaaactcatttgaaaggaaaaaaataattacaaattattgagagagaaatgagagagaaaatcattgtgattttcgtacactcaccgatttttagattacaactgcggattctttggattaggttgattgttagacagcaggttctacatatataatacttcaaattgtttgatgagatcgttaaaaagatatttgaagagagagataaatgtttttcattttctgttttatattttgggatttcatcttcttgtctctattgtatcaattgggggtctgttttgatttggctgtttgtagcacgtttcgatgcacttgttgaagactttcttgtaactttattgattatagtgaaattatttctttggtataaatgatccgtgatttttacccttgcattaagggatttttcactttaaacaaattgtgtctcttgtgcgcttttaatttctattttgctctctttatattttagcctgctccccaagtttggaccaatttatttctgctgcctatattactttgttattgtgttggtatattttcttcatcaaaaaatatccatttgtgctttaaattttgggtagttttgtttcttgctccacaaatttttgcagttttagtggaaaggcccaaccaatattctcaacattttgggccttcctcgaacaaggacccgtaagaatagaagtttctaaaatggacttgtaattggtg

SEQ ID NO: 69 - DNA, 23 bp, Glycine max
  source 1..23: mol_type = unassigned DNA
ggcaaggcacactgcggatgagg

SEQ ID NO: 70 - DNA, 23 bp, Glycine max
  source 1..23: mol_type = unassigned DNA
gtacgctggcgtcatgagggagg

SEQ ID NO: 71 - DNA, 23 bp, Glycine max
  source 1..23: mol_type = unassigned DNA
ggcggccggagacatgccggagg

SEQ ID NO: 72 - DNA, 23 bp, Glycine max
  source 1..23: mol_type = unassigned DNA
ggaaattgctgaattgtacgagg

SEQ ID NO: 73 - DNA, 23 bp, Glycine max
  source 1..23: mol_type = unassigned DNA
gatcttaggctctttgaacgagg

SEQ ID NO: 74 - DNA, 23 bp, Glycine max
  source 1..23: mol_type = unassigned DNA
gggaagcttgcgatcgggtgcgg

SEQ ID NO: 75 - DNA, 23 bp, Glycine max
  source 1..23: mol_type = unassigned DNA
gaatcggaaggagttgtcggcgg

SEQ ID NO: 76 - DNA, 23 bp, Glycine max
  source 1..23: mol_type = unassigned DNA
ggctgattctaccgcgaccgtgg

SEQ ID NO: 77 - protein, 1387 aa, synthetic construct
  REGION 1..1387: note = Synthetic
  REGION 1..10: note = MISC_FEATURE - nuclear localization sequence
  REGION 1378..1387: note = MISC_FEATURE - nuclear localization sequence
GSKKRRIKQDMDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSKKRRIKQD

SEQ ID NO: 78-79 - skipped (2 entries)

SEQ ID NO: 80 - DNA, 300 bp, synthetic construct
  misc_feature 1..300: note = Synthetic
  source 1..300: mol_type = other DNA
aagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggtt

SEQ ID NO: 81 - DNA, 300 bp, synthetic construct
  misc_feature 1..300: note = Synthetic
  source 1..300: mol_type = other DNA
aaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggatt

SEQ ID NO: 82 - DNA, 84 bp, synthetic construct
  misc_feature 1..84: note = Synthetic
  source 1..84: mol_type = other DNA
gttttagagctagaaatagcaagttaaaataaggctagtccgttatcaacttgaaaaagtggcaccgagtcggtgctttttttt

(19) United States
(12) Patent Application Publication
(10) Pub. No.: US 2026/0028635 A1
(43) Pub. Date: Jan. 29, 2026

(54) ENHANCED RECOMBINATION OF GENOMIC LOCI

(71) Applicant: Monsanto Technology LLC, St. Louis, MO (US)

(72) Inventors: David G. CALDWELL, St. Louis, MO (US); Ervin D. NAGY, Lake Saint Louis, MO (US)

(73) Assignee: Monsanto Technology LLC, St. Louis, MO (US)

(21) Appl. No.: 19/352,281

(22) Filed: Oct. 7, 2025

Related U.S. Application Data
(63) Continuation of application No. 15/753,928, now Pat. No. 12,460,217.
(60) Provisional application No. 62/208,405, filed on Aug. 21, 2015.

(51) Int. Cl. C12N 15/82

(52) U.S. Cl. CPC C12N 15/8213; C12N 15/8271

(57) ABSTRACT

The present disclosure provides methods to accelerate recombination at selected genomic loci, allowing recombination to occur, and selecting events with molecular variation within the selected loci. The accelerated recombination generates novel variations in gene clusters that are present in the plant or mammalian genomes.

### CROSS-REFERENCE TO RELATED APPLICATIONS

This application is a continuation of U.S. application Ser. No. 15/753,928, filed internationally on Aug. 19, 2016, which is a U.S. National Stage Application under 35 U.S.C. § 371 of International Application No. PCT/US2016/047748, filed on Aug. 19, 2016, which claims priority to U.S. Provisional Application No. 62/208,405 filed Aug. 21, 2015, each of which are herein incorporated by reference in their entirety.

### REFERENCE TO AN ELECTRONIC SEQUENCE LISTING

The contents of the electronic sequence listing (777052056401SeqList.xml; Size: 139,841 bytes; and Date of Creation: Oct. 6, 2025) is herein incorporated by reference in its entirety.

### FIELD

The present disclosure provides compositions and methods for enhancing recombination at preselected genomic loci by introducing site-specific genome modifications, allowing recombination to occur, and selecting events with molecular variation within the selected genomic loci.

### BACKGROUND

Genetic diversity underlies environmental adaptation. Currently, plant breeders are dependent on natural mechanisms of producing genetic diversity that rely on rare random mutation or recombination events for production of plants with desirable traits, such as resistance to new plant pathogen biotypes. Standard plant breeding is then used to integrate the desirable traits into select elite germplasm lines, though this is a slow and costly process involving multiple rounds of back crossing and selection. In some instances standard breeding techniques cannot overcome certain genetic linkages associated with unfavorable traits.

Therefore, there is a need for methods that will facilitate accelerated development of new loci for development of plants with improved environmental adaptation and agronomic traits. There is also a need in breeding programs to have methods to stimulate cis-chromosome exchange, sister chromosome exchange, or multiple chromosome exchange events within a single cell.

### BRIEF SUMMARY

Several embodiments relate to a method of generating a plant having a new array of tandemly duplicated genes, comprising contacting a plant cell with a first site-specific genome modification enzyme that introduces a genome modification in at least one target sequence of a first array of tandemly duplicated genes, thereby inducing recombination with a second array of tandemly duplicated genes, and selecting at least one plant comprising a new array of tandemly duplicated genes. In some embodiments, two new arrays of tandemly duplicated genes loci are generated. In some embodiments, the first array of tandemly duplicated genes and the second array of tandemly duplicated genes are on homologous chromosomes. In some embodiments, the first array of tandemly duplicated genes and the second array of tandemly duplicated genes are on non-homologous chromosomes. In some embodiments, the first array of tandemly duplicated genes and the second array of tandemly duplicated genes are on homoeologous chromosomes. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new array of tandemly duplicated genes has an increased number of genes compared to the first array of tandemly duplicated genes or the second array of tandemly duplicated genes. In some embodiments, the recombination is asymmetric and the new array of tandemly duplicated genes has a reduced number of genes compared to the first array of tandemly duplicated genes or the second array of tandemly duplicated genes. In some embodiments, the recombination is symmetric. In some embodiments, the recombination is symmetric, the first array of tandemly duplicated genes and second array of tandemly duplicated genes are heterologous and the new array of tandemly duplicated genes comprises a new combination of genes. In some embodiments, the target sequence is within a gene of the first array of tandemly duplicated genes. In some embodiments, the target sequence is within an intergenic region. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array. In some embodiments, the target sequence is in a genomic locus of the first array that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are in corresponding positions in the genome. In some embodiments, the target sequence is in a genomic locus of the first array that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are not in corresponding positions in the genome. In some embodiments, the first array of tandemly duplicated genes and the second array of tandemly duplicated genes are homologous. In some embodiments, the first array of tandemly duplicated genes and second array of tandemly duplicated genes are heterologous. In some embodiments, the first array of tandemly duplicated genes and second array of tandemly duplicated genes are homoeologous. In some embodiments, the first array of tandemly duplicated genes and second array of tandemly duplicated genes are paraologous. In some embodiments, the first array of tandemly duplicated genes and second array of tandemly duplicated genes are identical. In some embodiments, the first array of tandemly duplicated genes and the second array of tandemly duplicated genes are not identical. In some embodiments, the first array of tandemly duplicated genes is located in a first parental genome and the second array of tandemly duplicated genes is located in a second parental genome. In some embodiments, the first parental genome and the second parental genome are not sexually compatible. In some embodiments, the first parental genome and the second parental genome are different species. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the first parental genome and/or the second parental genome are haploid. In some embodiments, the first parental genome and/or the second parental genome are diploid. In some embodiments, the first array of tandemly duplicated genes, the second array of tandemly duplicated genes and the new array of tandemly duplicated genes encode NBS-LRR disease resistance proteins, pathogen recognition receptor (PRR) proteins, seed storage proteins, cell wall component extension proteins, F-box proteins, ABC transporters, or serine-threonine/tyrosine protein kinases. In some embodiments, the first array of tandemly duplicated genes, the second array of tandemly duplicated genes and the new array of tandemly duplicated genes encode ribosomal RNAs. In some embodiments, the at least one progeny comprising the new array of tandemly duplicated genes exhibits improved disease resistance compared to a plant comprising the first array of tandemly duplicated genes, a plant comprising the second array of tandemly duplicated genes, or a plant comprising the first array of tandemly duplicated genes and the second array of tandemly duplicated genes. In some embodiments, the new array of tandemly duplicated genes confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a plant having a new array of tandemly duplicated genes, comprising contacting a plant cell with a first site-specific genome modification enzyme that introduces a genome modification in at least one target sequence of a first array of tandemly duplicated genes and in at least one target sequence of a second array of tandemly duplicated genes, thereby inducing recombination between the first and second array of tandemly duplicated genes, and selecting at least one plant comprising a new array of tandemly duplicated genes. In some embodiments, two new arrays of tandemly duplicated genes loci are generated. In some embodiments, the first array of tandemly duplicated genes and the second array of tandemly duplicated genes are on homologous chromosomes. In some embodiments, the first array of tandemly duplicated genes and the second array of tandemly duplicated genes are on non-homologous chromosomes. In some embodiments, the first array of tandemly duplicated genes and the second array of tandemly duplicated genes are on homoeologous chromosomes. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new array of tandemly duplicated genes has an increased number of genes compared to the first array of tandemly duplicated genes or the second array of tandemly duplicated genes. In some embodiments, the recombination is asymmetric and the new array of tandemly duplicated genes has a reduced number of genes compared to the first array of tandemly duplicated genes or the second array of tandemly duplicated genes. In some embodiments, the recombination is symmetric. In some embodiments, the recombination is symmetric, the first array of tandemly duplicated genes and second array of tandemly duplicated genes are heterologous and the new array of tandemly duplicated genes comprises a new combination of genes. In some embodiments, the target sequence is genic. In some embodiments, the target sequence is within an intergenic region. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are in corresponding positions in the genome. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are not in corresponding positions in the genome. In some embodiments, the target sequence in the first array of tandemly duplicated genes has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the target sequence in the second array of tandemly duplicated genes. In some embodiments, the first array of tandemly duplicated genes and the second array of tandemly duplicated genes are homologous. In some embodiments, the first array of tandemly duplicated genes and second array of tandemly duplicated genes are heterologous. In some embodiments, the first array of tandemly duplicated genes and second array of tandemly duplicated genes are homoeologous. In some embodiments, the first array of tandemly duplicated genes and second array of tandemly duplicated genes are paraologous. In some embodiments, the first array of tandemly duplicated genes and second array of tandemly duplicated genes are identical. In some embodiments, the first array of tandemly duplicated genes and the second array of tandemly duplicated genes are not identical. In some embodiments, the first array of tandemly duplicated genes is located in a first parental genome and the second array of tandemly duplicated genes is located in a second parental genome. In some embodiments, the first parental genome and the second parental genome are not sexually compatible. In some embodiments, the first parental genome and the second parental genome are different species. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the first parental genome and/or the second parental genome are haploid. In some embodiments, the first parental genome and/or the second parental genome are diploid. In some embodiments, the first array of tandemly duplicated genes, the second array of tandemly duplicated genes and the new array of tandemly duplicated genes encode NBS-LRR disease resistance proteins, pathogen recognition receptor (PRR) proteins, seed storage proteins, cell wall component extension proteins, F-box proteins, ABC transporters, or serine-threonine/tyrosine protein kinases. In some embodiments, the first array of tandemly duplicated genes, the second array of tandemly duplicated genes and the new array of tandemly duplicated genes encode ribosomal RNAs. In some embodiments, the at least one progeny comprising the new array of tandemly duplicated genes exhibits improved disease resistance compared to a plant comprising the first array of tandemly duplicated genes, a plant comprising the second array of tandemly duplicated genes, or a plant comprising the first array of tandemly duplicated genes and the second array of tandemly duplicated genes. In some embodiments, the new array of tandemly duplicated genes confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a plant having a new array of tandemly duplicated genes, comprising contacting a plant cell with a first site-specific genome modification enzyme that introduces a genome modification in at least one target sequence of a first array of tandemly duplicated genes and a second site-specific genome modification enzyme that introduces a genome modification in at least one target sequence of a second array of tandemly duplicated genes, thereby inducing recombination between the first and second array of tandemly duplicated genes, and selecting at least one plant comprising a new array of tandemly duplicated genes. In some embodiments, two new arrays of tandemly duplicated genes loci are generated. In some embodiments, the first array of tandemly duplicated genes and the second array of tandemly duplicated genes are on homologous chromosomes. In some embodiments, the first array of tandemly duplicated genes and the second array of tandemly duplicated genes are on non-homologous chromosomes. In some embodiments, the first array of tandemly duplicated genes and the second array of tandemly duplicated genes are on homoeologous chromosomes. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new array of tandemly duplicated genes has an increased number of genes compared to the first array of tandemly duplicated genes or the second array of tandemly duplicated genes. In some embodiments, the recombination is asymmetric and the new array of tandemly duplicated genes has a reduced number of genes compared to the first array of tandemly duplicated genes or the second array of tandemly duplicated genes. In some embodiments, the recombination is symmetric. In some embodiments, the recombination is symmetric, the first array of tandemly duplicated genes and second array of tandemly duplicated genes are heterologous and the new array of tandemly duplicated genes comprises a new combination of genes. In some embodiments, the target sequence is genic. In some embodiments, the target sequence is within an intergenic region. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are in corresponding positions in the genome. In some embodiments, the target sequence in the first array of tandemly duplicated genes is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second array, where the genomic locus of the first array and the genomic locus of the first array are not in corresponding positions in the genome. In some embodiments, the target sequence in the first array of tandemly duplicated genes has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the target sequence in the second array of tandemly duplicated genes. In some embodiments, the first array of tandemly duplicated genes and the second array of tandemly duplicated genes are homologous. In some embodiments, the first array of tandemly duplicated genes and second array of tandemly duplicated genes are heterologous. In some embodiments, the first array of tandemly duplicated genes and second array of tandemly duplicated genes are homoeologous. In some embodiments, the first array of tandemly duplicated genes and second array of tandemly duplicated genes are paraologous. In some embodiments, the first array of tandemly duplicated genes and second array of tandemly duplicated genes are identical. In some embodiments, the first array of tandemly duplicated genes and the second array of tandemly duplicated genes are not identical. In some embodiments, the first array of tandemly duplicated genes is located in a first parental genome and the second array of tandemly duplicated genes is located in a second parental genome. In some embodiments, the first parental genome and the second parental genome are not sexually compatible. In some embodiments, the first parental genome and the second parental genome are different species. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the first parental genome and/or the second parental genome are haploid. In some embodiments, the first parental genome and/or the second parental genome are diploid. In some embodiments, the first array of tandemly duplicated genes, the second array of tandemly duplicated genes and the new array of tandemly duplicated genes encode NBS-LRR disease resistance proteins, pathogen recognition receptor (PRR) proteins, seed storage proteins, cell wall component extension proteins, F-box proteins, ABC transporters, or serine-threonine/tyrosine protein kinases. In some embodiments, the first array of tandemly duplicated genes, the second array of tandemly duplicated genes and the new array of tandemly duplicated genes encode ribosomal RNAs. In some embodiments, the at least one progeny comprising the new array of tandemly duplicated genes exhibits improved disease resistance compared to a plant comprising the first array of tandemly duplicated genes, a plant comprising the second array of tandemly duplicated genes, or a plant comprising the first array of tandemly duplicated genes and the second array of tandemly duplicated genes. In some embodiments, the new array of tandemly duplicated genes confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a corn plant having a new allele of an Rp1 disease resistance locus, comprising contacting a corn cell with a first site-specific genome modification enzyme that introduces a genome modification in at least one target sequence of a first Rp1 disease resistance locus, thereby inducing recombination with a second Rp1 disease resistance locus, and selecting at least one corn plant comprising a new allele of the Rp1 disease resistance locus. Several embodiments relate to a method of generating a corn plant having a new allele of an Rp1 disease resistance locus, comprising contacting a corn cell with a first site-specific genome modification enzyme that introduces a genome modification in at least one target sequence of a first Rp1 disease resistance locus and in at least one target sequence of a first Rp1 disease resistance locus, thereby inducing recombination between the first Rp1 disease resistance locus and the second Rp1 disease resistance locus, and selecting at least one corn plant comprising a new allele of the Rp1 disease resistance locus. Several embodiments relate to a method of generating a corn plant having a new allele of an Rp1 disease resistance locus, comprising contacting a corn cell with a first site-specific genome modification enzyme that introduces a genome modification in at least one target sequence of a first Rp1 disease resistance locus and contacting the corn cell with a second site-specific genome modification enzyme that introduces a genome modification in at least one target sequence of a second Rp1 disease resistance locus, thereby inducing recombination between the first Rp1 disease resistance locus and the second Rp1 disease resistance locus, and selecting at least one corn plant comprising a new allele of the Rp1 disease resistance locus. In some embodiments, two new alleles of the Rp1 disease resistance locus are generated. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new allele of the Rp1 disease resistance locus has an increased number of Rp1 genes compared to the first Rp1 disease resistance locus or the second Rp1 disease resistance locus. In some embodiments, the recombination is asymmetric and the new allele of the Rp1 disease resistance locus has a reduced number of RP1 genes compared to the first Rp1 disease resistance locus or the second Rp1 disease resistance locus. In some embodiments, the recombination is symmetric. In some embodiments, the recombination is symmetric, the first Rp1 disease resistance locus and second Rp1 disease resistance locus are heterologous and the new allele of the Rp1 disease resistance locus comprises a new combination of Rp1 genes. In some embodiments, the target sequence is within a gene. In some embodiments, the target sequence is within an intergenic region. In some embodiments, the target sequence is selected from one or more of the group comprising SEQ ID NO: 3, SEQ ID NO: 4, SEQ ID NO: 5, SEQ ID NO: 6, SEQ ID NO: 7, SEQ ID NO: 8, and SEQ ID NO: 9. In some embodiments, the target sequence in the first Rp1 disease resistance locus is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of the second Rp1 disease resistance locus. In some embodiments, the first Rp1 disease resistance locus and the second Rp1 disease resistance locus are homologous. In some embodiments, the first Rp1 disease resistance locus and second Rp1 disease resistance locus are heterologous. In some embodiments, the first Rp1 disease resistance locus and second Rp1 disease resistance locus are homoeologous. In some embodiments, the first Rp1 disease resistance locus and second Rp1 disease resistance locus are identical. In some embodiments, the first Rp1 disease resistance locus and second Rp1 disease resistance locus are not identical. In some embodiments, the first Rp1 disease resistance locus is located in a first parental genome and the second Rp1 disease resistance locus is located in a second parental genome. In some embodiments, the new allele of an Rp1 disease resistance locus confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), and Northern Corn Leaf Blight (Exserohilum turcicum). In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a soy plant having a new allele of an Rpp1 disease resistance locus, comprising contacting a soy cell with a first site-specific genome modification enzyme that introduces a genome modification in at least one target sequence of a first Rpp1 disease resistance locus, thereby inducing recombination with a second Rpp1 disease resistance locus, and selecting at least one soy plant comprising a new allele of the Rpp1 disease resistance locus. Several embodiments relate to a method of generating a soy plant having a new allele of an Rpp1 disease resistance locus, comprising contacting a soy cell with a first site-specific genome modification enzyme that introduces a genome modification in at least one target sequence of a first Rpp1 disease resistance locus and in at least one target sequence of a first Rpp1 disease resistance locus, thereby inducing recombination between the first Rpp1 disease resistance locus and the second Rpp1 disease resistance locus, and selecting at least one soy plant comprising a new allele of the Rpp1 disease resistance locus. Several embodiments relate to a method of generating a soy plant having a new allele of an Rpp1 disease resistance locus, comprising contacting a soy cell with a first site-specific genome modification enzyme that introduces a genome modification in at least one target sequence of a first Rpp1 disease resistance locus and contacting the soy cell with a second site-specific genome modification enzyme that introduces a genome modification in at least one target sequence of a second Rpp1 disease resistance locus, thereby inducing recombination between the first Rpp1 disease resistance locus and the second Rpp1 disease resistance locus, and selecting at least one soy plant comprising a new allele of the Rpp1 disease resistance locus. In some embodiments, two new alleles of the Rpp1 disease resistance locus are generated. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new allele of the Rpp1 disease resistance locus has an increased number of Rpp1 genes compared to the first Rpp1 disease resistance locus or the second Rpp1 disease resistance locus. In some embodiments, the recombination is asymmetric and the new allele of the Rpp1 disease resistance locus has a reduced number of RPP1 genes compared to the first Rpp1 disease resistance locus or the second Rpp1 disease resistance locus. In some embodiments, the recombination is symmetric. In some embodiments, the recombination is symmetric, the first Rpp1 disease resistance locus and second Rpp1 disease resistance locus are heterologous and the new allele of the Rpp1 disease resistance locus comprises a new combination of Rpp1 genes. In some embodiments, the target sequence is within a gene. In some embodiments, the target sequence is within an intergenic region. In some embodiments, the target sequence is selected from one or more of the group comprising SEQ ID NO: 12, SEQ ID NO: 13, SEQ ID NO: 14, SEQ ID NO: 15, SEQ ID NO: 16, SEQ ID NO: 17, and SEQ ID NO: 18. In some embodiments, the target sequence in the first Rpp1 disease resistance locus is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of the second Rpp1 disease resistance locus. In some embodiments, the first Rpp1 disease resistance locus and the second Rpp1 disease resistance locus are homologous. In some embodiments, the first Rpp1 disease resistance locus and second Rpp1 disease resistance locus are heterologous. In some embodiments, the first Rpp1 disease resistance locus and second Rpp1 disease resistance locus are homoeologous. In some embodiments, the first Rpp1 disease resistance locus and second Rpp1 disease resistance locus are identical. In some embodiments, the first Rpp1 disease resistance locus and second Rpp1 disease resistance locus are not identical. In some embodiments, the first Rpp1 disease resistance locus is located in a first parental genome and the second Rpp1 disease resistance locus is located in a second parental genome. In some embodiments, the new allele of an Rpp1 disease resistance locus confers resistance to one or more diseases selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a soy plant having a new allele of an Rps1 disease resistance locus, comprising contacting a soy cell with a first site-specific genome modification enzyme that introduces a genome modification in at least one target sequence of a first Rps1 disease resistance locus, thereby inducing recombination with a second Rps1 disease resistance locus, and selecting at least one soy plant comprising a new allele of the Rps1 disease resistance locus. Several embodiments relate to a method of generating a soy plant having a new allele of an Rps1 disease resistance locus, comprising contacting a soy cell with a first site-specific genome modification enzyme that introduces a genome modification in at least one target sequence of a first Rps1 disease resistance locus and in at least one target sequence of a first Rps1 disease resistance locus, thereby inducing recombination between the first Rps1 disease resistance locus and the second Rps1 disease resistance locus, and selecting at least one soy plant comprising a new allele of the Rps1 disease resistance locus. Several embodiments relate to a method of generating a soy plant having a new allele of an Rps1 disease resistance locus, comprising contacting a soy cell with a first site-specific genome modification enzyme that introduces a genome modification in at least one target sequence of a first Rps1 disease resistance locus and contacting the soy cell with a second site-specific genome modification enzyme that introduces a genome modification in at least one target sequence of a second Rps1 disease resistance locus, thereby inducing recombination between the first Rps1 disease resistance locus and the second Rps1 disease resistance locus, and selecting at least one soy plant comprising a new allele of the Rps1 disease resistance locus. In some embodiments, two new alleles of the Rps1 disease resistance locus are generated. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new allele of the Rps1 disease resistance locus has an increased number of Rps1 genes compared to the first Rps1 disease resistance locus or the second Rps1 disease resistance locus. In some embodiments, the recombination is asymmetric and the new allele of the Rps1 disease resistance locus has a reduced number of RPS1 genes compared to the first Rps1 disease resistance locus or the second Rps1 disease resistance locus. In some embodiments, the recombination is symmetric. In some embodiments, the recombination is symmetric, the first Rps1 disease resistance locus and second Rps1 disease resistance locus are heterologous and the new allele of the Rps1 disease resistance locus comprises a new combination of Rps1 genes. In some embodiments, the target sequence is within a gene. In some embodiments, the target sequence is within an intergenic region. In some embodiments, the target sequence is selected from one or more of the group comprising SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32. In some embodiments, the target sequence in the first Rps1 disease resistance locus is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of the second Rps1 disease resistance locus. In some embodiments, the first Rps1 disease resistance locus and the second Rps1 disease resistance locus are homologous. In some embodiments, the first Rps1 disease resistance locus and second Rps1 disease resistance locus are heterologous. In some embodiments, the first Rps1 disease resistance locus and second Rps1 disease resistance locus are homoeologous. In some embodiments, the first Rps1 disease resistance locus and second Rps1 disease resistance locus are identical. In some embodiments, the first Rps1 disease resistance locus and second Rps1 disease resistance locus are not identical. In some embodiments, the first Rps1 disease resistance locus is located in a first parental genome and the second Rps1 disease resistance locus is located in a second parental genome. In some embodiments, the new allele of an Rps1 disease resistance locus confers resistance to one or more diseases selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of generating a soy plant having a new allele of an Rhg1 soy cyst nematode resistance locus, comprising contacting a soy cell with a first site-specific genome modification enzyme that introduces a genome modification in at least one target sequence of a first Rhg1 soy cyst nematode resistance locus, thereby inducing recombination with a second Rhg1 soy cyst nematode resistance locus, and selecting at least one soy plant comprising a new allele of the Rhg1 soy cyst nematode resistance locus. Several embodiments relate to a method of generating a soy plant having a new allele of an Rhg1 soy cyst nematode resistance locus, comprising contacting a soy cell with a first site-specific genome modification enzyme that introduces a genome modification in at least one target sequence of a first Rhg1 soy cyst nematode resistance locus and in at least one target sequence of a first Rhg1 soy cyst nematode resistance locus, thereby inducing recombination between the first Rhg1 soy cyst nematode resistance locus and the second Rhg1 soy cyst nematode resistance locus, and selecting at least one soy plant comprising a new allele of the Rhg1 soy cyst nematode resistance locus. Several embodiments relate to a method of generating a soy plant having a new allele of an Rhg1 soy cyst nematode resistance locus, comprising contacting a soy cell with a first site-specific genome modification enzyme that introduces a genome modification in at least one target sequence of a first Rhg1 soy cyst nematode resistance locus and contacting the soy cell with a second site-specific genome modification enzyme that introduces a genome modification in at least one target sequence of a second Rhg1 soy cyst nematode resistance locus, thereby inducing recombination between the first Rhg1 soy cyst nematode resistance locus and the second Rhg1 soy cyst nematode resistance locus, and selecting at least one soy plant comprising a new allele of the Rhg1 soy cyst nematode resistance locus. In some embodiments, two new alleles of the Rhg1 soy cyst nematode resistance locus are generated. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric and the new allele of the Rhg1 soy cyst nematode resistance locus has an increased number of Rhg1 genes compared to the first Rhg1 soy cyst nematode resistance locus or the second Rhg1 soy cyst nematode resistance locus. In some embodiments, the recombination is asymmetric and the new allele of the Rhg1 soy cyst nematode resistance locus has a reduced number of Rhg1 genes compared to the first Rhg1 soy cyst nematode resistance locus or the second Rhg1 soy cyst nematode resistance locus. In some embodiments, the recombination is symmetric. In some embodiments, the recombination is symmetric, the first Rhg1 soy cyst nematode resistance locus and second Rhg1 soy cyst nematode resistance locus are heterologous and the new allele of the Rhg1 soy cyst nematode resistance locus comprises a new combination of Rhg1 genes. In some embodiments, the target sequence is within a gene. In some embodiments, the target sequence is within an intergenic region. In some embodiments, the target sequence is selected from one or more of the group comprising SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76. In some embodiments, the target sequence in the first Rhg1 soy cyst nematode resistance locus is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of the second Rhg1 soy cyst nematode resistance locus. In some embodiments, the first Rhg1 soy cyst nematode resistance locus and the second Rhg1 soy cyst nematode resistance locus are homologous. In some embodiments, the first Rhg1 soy cyst nematode resistance locus and second Rhg1 soy cyst nematode resistance locus are heterologous. In some embodiments, the first Rhg1 soy cyst nematode resistance locus and second Rhg1 soy cyst nematode resistance locus are homoeologous. In some embodiments, the first Rhg1 soy cyst nematode resistance locus and second Rhg1 soy cyst nematode resistance locus are identical. In some embodiments, the first Rhg1 soy cyst nematode resistance locus and second Rhg1 soy cyst nematode resistance locus are not identical. In some embodiments, the first Rhg1 soy cyst nematode resistance locus is located in a first parental genome and the second Rhg1 soy cyst nematode resistance locus is located in a second parental genome. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of providing a plant with improved disease resistance, comprising: (a) providing to one or more plant cells a site-specific genome modification enzyme that introduces a genome modification in at least one target sequence in a disease resistance locus; (b) screening for asymmetric recombination between disease-resistance loci on homologous chromosomes to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a site-specific genome modification enzyme that introduces a genome modification in at least one target sequence in a first disease resistance locus; (b) screening for recombination between the first disease-resistance locus and a second disease resistance locus, where the first and second disease resistance loci are on non-homologous chromosomes to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a site-specific genome modification enzyme that introduces a genome modification in at least one target sequence in a first disease resistance locus; (b) screening for recombination between the first disease-resistance locus and a second disease resistance locus, where the first and second disease resistance loci are on homoeologous chromosomes to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a site-specific genome modification enzyme that introduces a genome modification a first target sequence in a disease resistance locus and a second target sequence in a disease resistance locus, wherein the first target sequence and second target sequence are on homologous chromosomes; (b) screening for asymmetric recombination between disease-resistance loci on homologous chromosomes to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a site-specific genome modification enzyme that introduces a genome modification a first target sequence in a first disease resistance locus and a second target sequence in a second disease resistance locus, wherein the first disease resistance locus and second disease resistance locus are on non-homologous chromosomes; (b) screening for recombination between the disease-resistance loci to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a site-specific genome modification enzyme that introduces a genome modification a first target sequence in a first disease resistance locus and a second target sequence in a second disease resistance locus, wherein the first disease resistance locus and second disease resistance locus are on homoeologous chromosomes; (b) screening for recombination between the disease-resistance loci to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a first site-specific genome modification enzyme that introduces a genome modification a first target sequence in a disease resistance locus and a second site-specific genome modification enzyme that introduces a genome modification in a second target sequence in a disease resistance locus, wherein the first target sequence and second target sequence are on homologous chromosomes; (b) screening for asymmetric recombination between disease-resistance loci on homologous chromosomes to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a first site-specific genome modification enzyme that introduces a genome modification a first target sequence in a first disease resistance locus and a second site-specific genome modification enzyme that introduces a genome modification in a second target sequence in a second disease resistance locus, wherein the first disease resistance locus and second disease resistance locus are on non-homologous chromosomes; (b) screening for asymmetric recombination between the disease-resistance loci to identify plant cells comprising a recombinant disease resistance locus; (c) testing plants obtained from the plant cells identified in step (b) and their progeny for improved disease resistance; and (d) selecting the plant with improved disease resistance. Several embodiments relate to a method of providing a plant with improved disease resistance, comprising: (a) providing to one or more plant cells a first site-specific genome modification enzyme that introduces a genome modification a first target sequence in a first disease resistance locus and a second site-specific genome modification enzyme that introduces a genome modification in a second target sequence in a second disease resistance locus, wherein the first disease resistance locus and second disease resistance locus are on homoeologous chromosomes; (b) screening for asymmetric recombination between the disease-resistance loci to identify plant cells comprising a recombinant disease resistance locus; (c) testing plants obtained from the plant cells identified in step (b) and their progeny for improved disease resistance; and (d) selecting the plant with improved disease resistance. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombinant disease resistance locus has an increased number of genes compared to the disease resistance locus in either parent. In some embodiments, the recombinant disease resistance locus has reduced number of genes compared to the disease resistance locus in either parent. In some embodiments, the recombinant disease resistance locus has a different combination of genes compared to the disease resistance locus in either parent. In some embodiments, the target sequence is genic. In some embodiments, the target sequence is within an intergenic region. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome that is in a different position. In some embodiments, recombination is between homologous disease-resistance loci. In some embodiments, recombination is between heterologous disease-resistance loci. In some embodiments, recombination is between homoeologous disease-resistance loci. In some embodiments, recombination is between paraologous disease-resistance loci. In some embodiments, recombination is between identical disease-resistance loci. In some embodiments, the homologous chromosomes are from sexually incompatible parental genomes. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the homologous chromosomes are different plant species. In some embodiments, the first disease resistance locus is Rgh1 and the second disease resistance locus is Rgh4. In some embodiments, the disease resistance locus encodes NBS-LRR disease resistance proteins, pathogen recognition receptor (PRR) proteins, seed storage proteins, cell wall component extension proteins, F-box proteins, ABC transporters, or serine-threonine/tyrosine protein kinases. In some embodiments, the recombinant disease resistance locus confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of providing a corn plant with improved disease resistance, comprising: (a) providing to one or more plant cells a site-specific genome modification enzyme that introduces a genome modification in at least one target sequence in a Rp1 disease resistance locus; (b) screening for asymmetric recombination between Rp1 disease-resistance loci on homologous chromosomes to identify corn cells comprising a recombinant Rp1 disease resistance locus; (c) testing corn plants obtained from the corn cells identified in step (b) and their progeny for improved disease resistance; and (d) selecting the corn plant with improved resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), and Northern Corn Leaf Blight (Exserohilum turcicum). Several embodiments relate to a method of providing a corn plant with improved disease resistance, comprising: (a) providing to one or more corn cells a site-specific genome modification enzyme that introduces a genome modification a first target sequence in a Rp1 disease resistance locus and a second target sequence in the Rp1 disease resistance locus, wherein the first target sequence and second target sequence are on homologous chromosomes; (b) screening for asymmetric recombination between Rp1 disease-resistance loci on homologous chromosomes to identify plant cells comprising a recombinant Rp1 disease resistance locus; (c) testing plants obtained from the plant cells identified in step (b) and their progeny for improved disease resistance; and (d) selecting the corn plant with improved resistance to a disease selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), and Northern Corn Leaf Blight (Exserohilum turcicum). Several embodiments relate to a method of providing a corn plant with improved disease resistance, comprising: (a) providing to one or more plant cells a first site-specific genome modification enzyme that introduces a genome modification a first target sequence in a Rp1 disease resistance locus and a second site-specific genome modification enzyme that introduces a genome modification in a second target sequence in a Rp1 disease resistance locus, wherein the first target sequence and second target sequence are on homologous chromosomes; (b) screening for asymmetric recombination between Rp1 disease-resistance loci on homologous chromosomes to identify plant cells comprising a recombinant Rp1 disease resistance locus; (c) testing corn plants obtained from the corn cells identified in step (b) and their progeny for improved disease resistance; and (d) selecting the corn plant with improved resistance to a disease selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), and Northern Corn Leaf Blight (Exserohilum turcicum. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombinant Rp1 disease resistance locus has an increased number of genes compared to the Rp1 disease resistance locus in either parent. In some embodiments, the recombinant Rp1 disease resistance locus has reduced number of genes compared to the Rp1 disease resistance locus in either parent. In some embodiments, the recombinant Rp1 disease resistance locus has a different combination of genes compared to the Rp1 disease resistance locus in either parent. In some embodiments, the target sequence is genic. In some embodiments, the target sequence is within an intergenic region. In some embodiments, the target sequence is selected from one or more of the group comprising SEQ ID NO: 3, SEQ ID NO: 4, SEQ ID NO: 5, SEQ ID NO: 6, SEQ ID NO: 7, SEQ ID NO: 8, and SEQ ID NO: 9. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in a different position on the homologous chromosome. In some embodiments, recombination is between homologous Rp1 disease-resistance loci. In some embodiments, recombination is between heterologous Rp1 disease-resistance loci. In some embodiments, recombination is between homoeologous Rp1 disease-resistance loci. In some embodiments, recombination is between paraologous Rp1 disease-resistance loci. In some embodiments, recombination is between identical Rp1 disease-resistance loci. In some embodiments, the homologous chromosomes are from sexually incompatible parental genomes. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the homologous chromosomes are different plant species. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of providing a soy plant with improved disease resistance, comprising: (a) providing to one or more plant cells a site-specific genome modification enzyme that introduces a genome modification in at least one target sequence in a Rpp1 disease resistance locus; (b) screening for asymmetric recombination between Rpp1 disease-resistance loci on homologous chromosomes to identify soy cells comprising a recombinant Rpp1 disease resistance locus; (c) testing soy plants obtained from the soy cells identified in step (b) and their progeny for improved disease resistance; and (d) selecting the soy plant with improved resistance to one or more diseases selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. Several embodiments relate to a method of providing a soy plant with improved disease resistance, comprising: (a) providing to one or more soy cells a site-specific genome modification enzyme that introduces a genome modification a first target sequence in a Rpp1 disease resistance locus and a second target sequence in the Rpp1 disease resistance locus, wherein the first target sequence and second target sequence are on homologous chromosomes; (b) screening for asymmetric recombination between Rpp1 disease-resistance loci on homologous chromosomes to identify plant cells comprising a recombinant Rpp1 disease resistance locus; (c) testing plants obtained from the plant cells identified in step (b) and their progeny for improved disease resistance; and (d) selecting the soy plant with improved resistance to a disease selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. Several embodiments relate to a method of providing a soy plant with improved disease resistance, comprising: (a) providing to one or more plant cells a first site-specific genome modification enzyme that introduces a genome modification a first target sequence in a Rpp1 disease resistance locus and a second site-specific genome modification enzyme that introduces a genome modification in a second target sequence in a Rpp1 disease resistance locus, wherein the first target sequence and second target sequence are on homologous chromosomes; (b) screening for asymmetric recombination between Rpp1 disease-resistance loci on homologous chromosomes to identify plant cells comprising a recombinant Rpp1 disease resistance locus; (c) testing soy plants obtained from the soy cells identified in step (b) and their progeny for improved disease resistance; and (d) selecting the soy plant with improved resistance to a disease selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombinant Rpp1 disease resistance locus has an increased number of genes compared to the Rpp1 disease resistance locus in either parent. In some embodiments, the recombinant Rpp1 disease resistance locus has reduced number of genes compared to the Rpp1 disease resistance locus in either parent. In some embodiments, the recombinant Rpp1 disease resistance locus has a different combination of genes compared to the Rpp1 disease resistance locus in either parent. In some embodiments, the target sequence is genic. In some embodiments, the target sequence is within an intergenic region. In some embodiments, the target sequence is selected from one or more of the group comprising SEQ ID NO: 12, SEQ ID NO: 13, SEQ ID NO: 14, SEQ ID NO: 15, SEQ ID NO: 16, SEQ ID NO: 17, and SEQ ID NO: 18. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in a different position on the homologous chromosome. In some embodiments, recombination is between homologous Rpp1 disease-resistance loci. In some embodiments, recombination is between heterologous Rpp1 disease-resistance loci. In some embodiments, recombination is between homoeologous Rpp1 disease-resistance loci. In some embodiments, recombination is between paraologous Rpp1 disease-resistance loci. In some embodiments, recombination is between identical Rpp1 disease-resistance loci. In some embodiments, the homologous chromosomes are from sexually incompatible parental genomes. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the homologous chromosomes are different plant species. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of providing a soy plant with improved disease resistance, comprising: (a) providing to one or more plant cells a site-specific genome modification enzyme that introduces a genome modification in at least one target sequence in a Rps1 disease resistance locus; (b) screening for asymmetric recombination between Rps1 disease-resistance loci on homologous chromosomes to identify soy cells comprising a recombinant Rps1 disease resistance locus; (c) testing soy plants obtained from the soy cells identified in step (b) and their progeny for improved disease resistance; and (d) selecting the soy plant with improved resistance to one or more diseases selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. Several embodiments relate to a method of providing a soy plant with improved disease resistance, comprising: (a) providing to one or more soy cells a site-specific genome modification enzyme that introduces a genome modification a first target sequence in a Rps1 disease resistance locus and a second target sequence in the Rps1 disease resistance locus, wherein the first target sequence and second target sequence are on homologous chromosomes; (b) screening for asymmetric recombination between Rps1 disease-resistance loci on homologous chromosomes to identify plant cells comprising a recombinant Rps1 disease resistance locus; (c) testing plants obtained from the plant cells identified in step (b) and their progeny for improved disease resistance; and (d) selecting the soy plant with improved resistance to a disease selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. Several embodiments relate to a method of providing a soy plant with improved disease resistance, comprising: (a) providing to one or more plant cells a first site-specific genome modification enzyme that introduces a genome modification a first target sequence in a Rps1 disease resistance locus and a second site-specific genome modification enzyme that introduces a genome modification in a second target sequence in a Rps1 disease resistance locus, wherein the first target sequence and second target sequence are on homologous chromosomes; (b) screening for asymmetric recombination between Rps1 disease-resistance loci on homologous chromosomes to identify plant cells comprising a recombinant Rps1 disease resistance locus; (c) testing soy plants obtained from the soy cells identified in step (b) and their progeny for improved disease resistance; and (d) selecting the soy plant with improved resistance to a disease selected from Sudden Death Syndrome (SDS), Phytophthora Root Rot, Phytophthora Stem Rot, Fusarium Root Rot, Rhizoctonia Root Rot, Charcoal Rot, Soybean Cyst Nematode (SCN), Sclerotinia Stem Rot (White Mold), Brown Stem Rot (BSR), Pod and Stem Blight, Stem Canker, Anthracnose, Green Stem Syndrome, Soybean Rust, Septoria Brown Spot, Bacterial Blight, Downy Mildew, Cercospora Leaf Blight, Frogeye Leaf Spot, Powdery Mildew, Soybean Mosaic Virus and Bean Pod Mottle Virus. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombinant Rps1 disease resistance locus has an increased number of genes compared to the Rps1 disease resistance locus in either parent. In some embodiments, the recombinant Rps1 disease resistance locus has reduced number of genes compared to the Rps1 disease resistance locus in either parent. In some embodiments, the recombinant Rps1 disease resistance locus has a different combination of genes compared to the Rps1 disease resistance locus in either parent. In some embodiments, the target sequence is genic. In some embodiments, the target sequence is within an intergenic region. In some embodiments, the target sequence is selected from one or more of the group comprising SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in a different position on the homologous chromosome. In some embodiments, recombination is between homologous Rps1 disease-resistance loci. In some embodiments, recombination is between heterologous Rps1 disease-resistance loci. In some embodiments, recombination is between homoeologous Rps1 disease-resistance loci. In some embodiments, recombination is between paraologous Rps1 disease-resistance loci. In some embodiments, recombination is between identical Rps1 disease-resistance loci. In some embodiments, the homologous chromosomes are from sexually incompatible parental genomes. In some embodiments, the homologous chromosomes are different plant species. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of providing a soy plant with improved nematode resistance, comprising: (a) providing to one or more plant cells a site-specific genome modification enzyme that introduces a genome modification in at least one target sequence in a Rhg1 soy cyst nematode resistance locus; (b) screening for asymmetric recombination between Rhg1 soy cyst nematode resistance loci on homologous chromosomes to identify soy cells comprising a recombinant Rhg1 soy cyst nematode resistance locus; (c) testing soy plants obtained from the soy cells identified in step (b) and their progeny for improved nematode resistance; and (d) selecting the soy plant with improved nematode resistance. Several embodiments relate to a method of providing a soy plant with improved nematode resistance, comprising: (a) providing to one or more soy cells a site-specific genome modification enzyme that introduces a genome modification a first target sequence in a Rhg1 soy cyst nematode resistance locus and a second target sequence in the Rhg1 soy cyst nematode resistance locus, wherein the first target sequence and second target sequence are on homologous chromosomes; (b) screening for asymmetric recombination between Rhg1 soy cyst nematode resistance loci on homologous chromosomes to identify plant cells comprising a recombinant Rhg1 soy cyst nematode resistance locus; (c) testing plants obtained from the plant cells identified in step (b) and their progeny for improved nematode resistance; and (d) selecting the soy plant with improved nematode resistance. Several embodiments relate to a method of providing a soy plant with improved nematode resistance, comprising: (a) providing to one or more plant cells a first site-specific genome modification enzyme that introduces a genome modification a first target sequence in a Rhg1 soy cyst nematode resistance locus and a second site-specific genome modification enzyme that introduces a genome modification in a second target sequence in a Rhg1 soy cyst nematode resistance locus, wherein the first target sequence and second target sequence are on homologous chromosomes; (b) screening for asymmetric recombination between Rhg1 soy cyst nematode resistance loci on homologous chromosomes to identify soy cells comprising a recombinant Rhg1 soy cyst nematode resistance locus; (c) testing soy plants obtained from the soy cells identified in step (b) and their progeny for improved nematode resistance; and (d) selecting the soy plant with improved nematode resistance. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombinant Rhg1 soy cyst nematode resistance locus has an increased number of genes compared to the Rhg1 soy cyst nematode resistance locus in either parent. In some embodiments, the recombinant Rhg1 soy cyst nematode resistance locus has reduced number of genes compared to the Rhg1 soy cyst nematode resistance locus in either parent. In some embodiments, the recombinant Rhg1 soy cyst nematode resistance locus has a different combination of genes compared to the Rhg1 soy cyst nematode resistance locus in either parent. In some embodiments, the target sequence is genic. In some embodiments, the target sequence is within an intergenic region. In some embodiments, the target sequence is selected from one or more of the group comprising SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in a different position on the homologous chromosome. In some embodiments, recombination is between homologous Rhg1 soy cyst nematode resistance loci. In some embodiments, recombination is between heterologous Rhg1 soy cyst nematode resistance loci. In some embodiments, recombination is between homoeologous Rhg1 soy cyst nematode resistance loci. In some embodiments, recombination is between paraologous Rhg1 soy cyst nematode resistance loci. In some embodiments, recombination is between identical Rhg1 soy cyst nematode resistance loci. In some embodiments, the homologous chromosomes are from sexually incompatible parental genomes. In some embodiments, the homologous chromosomes are different plant species. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

A method of generating a plant with an altered disease resistance locus from an inbred line, comprising providing a site-specific genome modification enzyme to a plant cell, wherein the site-specific genome modification enzyme introduces a genome modification at least one target sequence in a disease resistance locus thereby inducing asymmetric recombination between homologous chromosomes within the disease resistance locus and growing the plant with the altered disease resistance locus from the plant cell. A method of generating a plant with an altered disease resistance locus from an inbred line, comprising providing a first site-specific genome modification enzyme and a second site-specific genome modification enzyme to a plant cell, wherein the first site-specific genome modification enzyme introduces a genome modification at a first target sequence in a disease resistance locus and the second site-specific genome modification enzyme introduces a genome modification at a second target sequence in a disease resistance locus thereby inducing asymmetric recombination between the first and second target sequences on homologous chromosomes and growing the plant with the altered disease resistance locus from the plant cell. A method of generating a plant with an altered disease resistance locus from an inbred line, comprising providing a site-specific genome modification enzyme to a plant cell, wherein the site-specific genome modification enzyme introduces a genome modification at least two target sequences in a disease resistance locus thereby inducing deletion of a sequence in the disease resistance locus and growing the plant with the altered disease resistance locus from the plant cell. In some embodiments, the plant with the altered disease resistance locus does not require backcrossing to achieve genetic identity. In some embodiments, the plant with the altered disease resistance locus has improved disease resistance compared a plant of the inbred line without the altered disease resistance locus. In some embodiments, disease resistance locus encodes one or more nucleotide-binding site leucine-rich repeat (NBS-LRR) disease resistance proteins. In some embodiments, the plant is a corn plant and the disease resistance locus is Rp1. In some embodiments, the plant is a soybean plant and the disease resistance locus is Rpp1. In some embodiments, the plant is a soybean plant and the disease resistance locus is Rps1. In some embodiments, the plant is a soybean plant and the disease resistance locus is Rhg1. In some embodiments, the plant is a soybean plant and the disease resistance locus is Rgh4. In some embodiments, the plant with the altered disease resistance locus has improved resistance to one or more diseases selected from the group consisting of Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the altered disease resistance locus has an increased number of genes compared to the disease resistance locus in either parental genome. In some embodiments, the altered disease resistance locus has a reduced number of genes compared to the disease resistance locus in either parental genome. In some embodiments, the altered disease resistance locus has a new combination of genes compared to the disease resistance locus in either parental genome. In some embodiments, the disease resistance loci in the parental genomes are identical. In some embodiments, the target sequence is genic. In some embodiments, the target sequence is within an intergenic region. In some embodiments, the target sequence in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in the disease resistance locus on the homologous chromosome. In some embodiments, the target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus in the disease resistance locus on the homologous chromosome, where the target sequence and the genomic locus in the disease resistance locus on the homologous chromosome are in different positions in the genome. In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. In some embodiments, the disease resistance locus Rp1, and the target sequence is selected from one or more of the group consisting of SEQ ID NO: 3, SEQ ID NO: 4, SEQ ID NO: 5, SEQ ID NO: 6, SEQ ID NO: 7, SEQ ID NO: 8, and SEQ ID NO: 9. In some embodiments, the disease resistance locus Rpp1, and the target sequence is selected from one or more of the group consisting of SEQ ID NO: 12, SEQ ID NO: 13, SEQ ID NO: 14, SEQ ID NO: 15, SEQ ID NO: 16, SEQ ID NO: 17, and SEQ ID NO: 18. In some embodiments, the disease resistance locus Rps1, and the target sequence is selected from one or more of the group consisting of SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, and SEQ ID NO: 32. In some embodiments, the disease resistance locus Rhg1, and the target sequence is selected from one or more of the group consisting of SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76. Several embodiments relate to a plant, plant cell or a seed of a plant produced by according to the aforementioned methods.

Several embodiments relate to a method of enhancing recombination at selected genomic loci, comprising providing to a plant cell at least one site-specific genome modification enzyme that introduces genome modification in a first genomic locus, thereby inducing recombination between the first genomic locus and a second genomic locus, wherein the at least one site-specific genome modification enzyme does not introduce a genome modification at the second genomic locus, and selecting at least one plant cell comprising a recombination event between the first genomic locus and the second genomic locus. Several embodiments relate to a method of enhancing recombination at selected genomic loci, comprising providing to a plant cell at least one site-specific genome modification enzyme that introduces genome modification at a first genomic locus and a second genomic locus, thereby inducing recombination between the first genomic locus and the second genomic locus, and selecting at least one plant cell comprising a recombination event between the first genomic locus and the second genomic locus. Several embodiments relate to a method of enhancing recombination at selected genomic loci, comprising providing to a cell a first site-specific genome modification enzyme that introduces a genome modification at a first genomic locus and a second site-specific genome modification enzyme that introduces a genome modification at a second genomic locus, thereby inducing recombination between the first genomic locus and the second genomic locus, and selecting at least one progeny comprising a recombination event between the first genomic locus and the second genomic locus. In some embodiments the first and second genomic loci are in cis. In some embodiments, the first and second genomic loci are in trans. In some embodiments, the first and second genomic loci are homologs. In some embodiments, the first and second genomic loci are paraologs. In some embodiments, the first and second genomic loci are homeologs. In some embodiments, the first and second genomic loci are identical. In some embodiments, the first genomic locus and the second genomic locus are on homologous chromosomes. In some embodiments, the first genomic locus and the second genomic locus are on non-homologous chromosomes. In some embodiments, the first genomic locus and the second genomic locus are on homoeologous chromosomes. In some embodiments, the first and second genomic loci share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity. In some embodiments, the first genomic locus and the second genomic locus are located on homologous chromosomes. In some embodiments, the first genomic locus and the second genomic locus are located on non-homologous chromosomes. In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the recombination is asymmetric. In some embodiments, the recombination is symmetric. In some embodiments, the first target sequence and/or the second target sequence is genic. In some embodiments, the first target sequence and/or the second target sequence is within an intergenic region. In some embodiments, the first target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus containing the second target sequence. In some embodiments, the first target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus containing the second target sequence, wherein the genomic locus containing the first target sequence and the genomic locus containing the second target sequence are in corresponding positions in the genome. In some embodiments, the first target sequence is in a genomic locus that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus containing the second target sequence, wherein the genomic locus containing the first target sequence and the genomic locus containing the second target sequence are not in corresponding positions in the genome. In some embodiments, the first target sequence has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the second target sequence. In some embodiments, one or more of the first genomic locus and the second genomic locus comprise one or more genomic regions selected independently from the group consisting of a gene, an array of tandemly duplicated genes, an enhancer, a suppressor, a promoter, a termination sequence, a splice acceptor sequence, a splice donor sequence, an intron, an exon, an siRNA, and a quantitative trait locus (QTL). In some embodiments, progeny of the one plant cell comprising the recombination event between the first genomic locus and the second genomic locus exhibit resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis). In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of introgressing a genomic locus of interest into a selected germplasm, comprising generating a plant cell comprising a first parental genome comprising the genomic locus of interest and a second parental genome comprising the selected germplasm, providing to the plant cell a first site-specific genome modification enzyme that introduces genome modification in the first parental genome at a target sequence adjacent to the genomic locus of interest, thereby inducing recombination between the first parental genome and the second parental genome, and selecting at least one progeny comprising at least one recombinant chromosome comprising the selected germplasm and the genomic locus of interest. Several embodiments relate to a method of introgressing a genomic locus of interest into a selected germplasm, comprising generating a plant cell comprising a first parental genome comprising the genomic locus of interest and a second parental genome comprising the selected germplasm, providing to the plant cell a first site-specific genome modification enzyme that introduces genome modification in the first parental genome at a target sequence adjacent to the genomic locus of interest and a genome modification at a target site in the second parental genome, thereby inducing recombination between the first parental genome and the second parental genome, and selecting at least one progeny comprising at least one recombinant chromosome comprising the selected germplasm and the genomic locus of interest. Several embodiments relate to a method of introgressing a genomic locus of interest into a selected germplasm, comprising generating a plant cell comprising a first parental genome comprising the genomic locus of interest and a second parental genome comprising the selected germplasm, providing to the plant cell a first site-specific genome modification enzyme that introduces genome modification in the first parental genome at a target sequence adjacent to the genomic locus of interest and a second site-specific genome modification enzyme that introduces a genome modification in the first parental genome at a second target sequence adjacent to the genomic locus, wherein the second target sequence is on opposite side of the genome genomic locus of interest from the target sequence of the first site-specific genome modification enzyme, thereby inducing recombination between the first parental genome and the second parental genome, and selecting at least one plant cell comprising at least one recombinant chromosome comprising the selected germplasm and the genomic locus of interest. Several embodiments relate to a method of introgressing a genomic locus of interest into a selected germplasm, comprising generating a plant cell comprising a first parental genome comprising the genomic locus of interest and a second parental genome comprising the selected germplasm, providing to the plant cell a first site-specific genome modification enzyme that introduces genome modification in the first parental genome at a target sequence adjacent to the genomic locus of interest and a genome modification at a target site in the second parental genome and further introducing into the plant cell a second site-specific genome modification enzyme that introduces a genome modification in the first parental genome at a second target sequence adjacent to the genomic locus, wherein the second target sequence is on opposite side of the genome genomic locus of interest from the target sequence of the first site-specific genome modification enzyme, thereby inducing recombination between the first parental genome and the second parental genome, and selecting at least one plant cell comprising at least one recombinant chromosome comprising the selected germplasm and the genomic locus of interest. In some embodiments, the second site-specific genome modification enzyme introduces a genome modification at a target sequence in the second parental genome. In some embodiments, the recombination is asymmetric. In some embodiments, the recombination is symmetric. In some embodiments, the genomic locus of interest comprises one or more genomic regions selected independently from the group consisting of a gene, an array of tandemly duplicated genes, a multigene family, an enhancer, a suppressor, a promoter, a termination sequence, a splice acceptor sequence, a splice donor sequence, an intron, an exon, an siRNA, a sequence encoding a non-coding RNA, a microRNA, a transgene, and a quantitative trait locus (QTL). In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the target sequence is genic. In some embodiments, the target sequence is within an intergenic region. In some embodiments, the target sequence is in a genomic locus of the first parental genome that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second parental genome. In some embodiments, the target sequence is in a genomic locus of the first parental genome that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second parental genome, wherein the genomic locus of the first parental genome and the genomic locus of the second parental genome are located in corresponding positions. In some embodiments, the target sequence is in a genomic locus of the first parental genome that is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of a genomic locus of the second parental genome, wherein the genomic locus of the first parental genome and the genomic locus of the second parental genome are not located in corresponding positions, leading to asymmetric recombination. In some embodiments, the first parental genome and the second parental genome are not sexually compatible. In some embodiments, the first parental genome and the second parental genome are different species. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the first parental genome and/or the second parental genome are haploid. In some embodiments, the first parental genome and/or the second parental genome are diploid. In some embodiments, the genomic locus of interest is Rp1 disease resistance locus. In some embodiments, the genomic locus of interest is Rpp1 disease resistance locus. In some embodiments, the genomic locus of interest is Rps1 disease resistance locus. In some embodiments, the genomic locus of interest is Rhg1 disease resistance locus. In some embodiments, the genomic locus of interest is Rgh4 disease resistance locus. In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods.

Several embodiments relate to a method of removing linkage drag, comprising generating a plant cell comprising a first parental genome and a second parental genome, wherein the first parental genome comprises a genomic locus of interest linked in cis to an undesirable genomic locus, providing to the cell a first site-specific genome modification enzyme that introduces a genome modification between the genomic locus of interest and the undesirable genomic locus, thereby inducing recombination between the first parental genome and the second parental genome and unlinking the genomic locus of interest and the undesirable locus, and selecting at least one progeny comprising the genomic locus of interest. Several embodiments relate to a method of removing linkage drag, comprising generating a plant cell comprising a first parental genome and a second parental genome, wherein the first parental genome comprises a genomic locus of interest linked in cis to an undesirable genomic locus, providing to the cell a first site-specific genome modification enzyme that introduces a first genome modification between the genomic locus of interest and the undesirable genomic locus and a second genome modification on opposite side of the undesirable genomic locus from the first genome modification, thereby inducing recombination between the first parental genome and the second parental genome and removing the undesirable locus while maintaining the germplasm of the first parental genome distal to the second genome modification, and selecting at least one progeny comprising the genomic locus of interest. In some embodiments, the second site-specific genome modification enzyme introduces a genome modification at a target sequence in the second parental genome. In some embodiments, the recombination is asymmetric. In some embodiments, the recombination is symmetric. In some embodiments, the genomic locus of interest comprises one or more genomic regions selected independently from the group consisting of a gene, an array of tandemly duplicated genes, a multigene family, an enhancer, a suppressor, a promoter, a termination sequence, a splice acceptor sequence, a splice donor sequence, an intron, an exon, an siRNA, a sequence encoding a non-coding RNA, a microRNA, a transgene, and a quantitative trait locus (QTL). In some embodiments, the genome modification is a double strand break (DSB). In some embodiments, the genome modification is a single strand break. In some embodiments, the genome modification is a recombinase-mediated DNA exchange reaction. In some embodiments, the genome modification is a transposase-mediated DNA exchange reaction. In some embodiments, the genome modification occurs at the beginning of meiosis. In some embodiments, the first parental genome and the second parental genome are not sexually compatible. In some embodiments, the first parental genome and the second parental genome are different species. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the first parental genome and/or the second parental genome are haploid. In some embodiments, the first parental genome and/or the second parental genome are diploid. In some embodiments, the genomic locus of interest is Rp1 disease resistance locus. In some embodiments, the genomic locus of interest is Rpp1 disease resistance locus. In some embodiments, the genomic locus of interest is Rps1 disease resistance locus. In some embodiments, the genomic locus of interest is Rhg1 disease resistance locus. In some embodiments, the genomic locus of interest is Rhg4 disease resistance locus. In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. Several embodiments relate to a plant, plant cell or a seed of a plant produced by according to the aforementioned methods. Several embodiments relate to a method of coupling genomic loci in repulsion, comprising generating a plant cell comprising a first parental genome comprising a first genomic locus and a second parental genome comprising a second genomic locus, wherein the first genomic locus and the second genetic locus are in repulsion, providing to the cell a first site-specific genome modification enzyme that introduces a genome modification adjacent to the first genomic locus, thereby inducing recombination between the first parental genome and the second parental genome, and selecting at least one plant cell comprising the first genomic locus and the second genomic locus on the same chromosome. In some embodiments, the first genomic locus and the second genomic locus are located on homologous chromosomes. In some embodiments, the first parental genome and the second parental genome are not sexually compatible. In some embodiments, the first parental genome and the second parental genome are different species. In some embodiments, the first genomic locus of interest and/or the second genomic locus of interest comprises one or more genomic regions selected independently from the group consisting of a gene, an array of tandemly duplicated genes, an enhancer, a suppressor, a promoter, a termination sequence, a splice acceptor sequence, a splice donor sequence, an intron, an exon, an siRNA, and a quantitative trait locus (QTL). In some embodiments, the first parental genome and/or the second parental genome are haploid. In some embodiments, the first parental genome and/or the second parental genome are diploid. In some embodiments, the first parental genome is Triticum aestivum (wheat) and the second parental genome is selected from Aegilops ovate, Ae. biuncialis, Ae. triuncialis, Ae. quarrosa, Secale cereal, Triticum dicoccoides, Triticum dicoccum and Triticum durum. In some embodiments, the first parental genome is selected from Aegilops ovate, Ae. biuncialis, Ae. triuncialis, Ae. quarrosa, Secale cereal, Triticum dicoccoides, Triticum dicoccum and Triticum durum and the second parental genome is Triticum aestivum (wheat). In some embodiments, the first parental genome is Gossypium hirsutum (cotton) and the second parental genome is selected from G. sturtii, G. davidsonii, G. arboretum and G. raimondii. In some embodiments, the first parental genome is selected from G. sturtii, G. davidsonii, G. arboretum and G. raimondii and the second parental genome is Gossypium hirsutum (cotton). In some embodiments, the genomic locus of interest is Rp1 disease resistance locus. In some embodiments, the first genomic locus of interest and/or the second genomic locus of interest is Rpp1 disease resistance locus. In some embodiments, the first genomic locus of interest and/or the second genomic locus of interest is Rps1 disease resistance locus. In some embodiments, the first genomic locus of interest and/or the second genomic locus of interest Rhg1 disease resistance locus. In some embodiments, the first genomic locus of interest and/or the second genomic locus of interest Rhg4 disease resistance locus. In some embodiments, the first genomic locus of interest is Rhg1 and the second genomic locus of interest Rhg4. In some embodiments, the plant is a maize plant. In some embodiments, the plant is a soybean plant. In some embodiments, the plant is a cotton plant. In some embodiments, the plant is a wheat plant. In some embodiments, the plant is a sorghum plant. In some embodiments, the plant is a canola plant. In some embodiments, the site-specific genome modification enzyme is an endonuclease. In some embodiments, the site-specific genome modification enzyme is an endonuclease selected from a meganuclease, a zinc finger nuclease, a transcription activator-like effector nuclease (TALEN), an Argonaute, an RNA-guided endonuclease, a type I CRISPR-Cas system, type II CRISPR-Cas system or a type III CRISPR-Cas system. In some embodiments, the site-specific genome modification enzyme is a CRISPR associate protein selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease. In some embodiments, the site-specific genome modification enzyme is a recombinase. In some embodiments, the site-specific genome modification enzyme is an RNA-guided recombinase. In some embodiments, the site-specific genome modification enzyme is a fusion protein comprising a recombinase and a CRISPR associated protein. In some embodiments, the recombinase is a tyrosine recombinase attached to a DNA recognition motif, or a serine recombinase attached to a DNA recognition motif. In some embodiments, the recombinase is a Cre recombinase, a Flp recombinase, a Tnp1 recombinase, a PhiC31 integrase, an R4 integrase, or a TP-901 integrase. In some embodiments, the site-specific genome modification enzyme is a transposase attached to a DNA binding domain. In some embodiments, one or more of the first parental genome and the second parental genome are from an elite germplasm line. Several embodiments relate to a plant, plant cell or a seed of a plant produced by according to the aforementioned methods.

Several embodiments relate to a method of generating a new array of tandemly duplicated genes, comprising contacting a cell with a site-specific genome modification enzyme that cleaves at least one target sequence in a first array of tandemly duplicated genes thereby inducing asymmetric recombination with a homologous sequence of a second array of tandemly duplicated genes and selecting at least one progeny comprising a new array of tandemly duplicated genes. In some embodiments, the first and second arrays of tandemly duplicated genes are identical. In other embodiments, the first and second arrays of tandemly duplicated genes are different. In some embodiments, the asymmetric recombination generates two new arrays of tandemly duplicated genes, depending on the recombination site. In some embodiments, the asymmetric recombination results in a deletion in at least one of the tandemly duplicated genes. In some embodiments, the cell is a plant cell. In a further embodiment, the plant cell is obtained from a plant selected from an inbred plant or a hybrid plant. In other embodiments, the cell is a mammalian cell.

In one aspect, the site-specific genome modification enzyme is selected from an endonuclease, a recombinase, a transposase, a helicase, or any combination thereof. In a further aspect, the endonuclease is selected from a meganuclease, a zinc-finger nuclease, a TALEN, a nucleic acid guided endonuclease, an Argonaute, a CRISPR/Cpf1 system and a CRISPR/Cas9 system.

In one aspect, the tandemly duplicated genes encode proteins selected from NBS-LRR disease resistance proteins, pathogen recognition receptor (PRR) proteins, seed storage proteins, the cell wall component extension proteins, F-box proteins, ABC transporters, immunoglobulins, serine-threonine/tyrosine protein kinases, and ribosomal RNAs. In the case of Rhg1, the tandem repeats are composed of three genes: a putative amino acid transporter, an alpha-SNAP protein, and a wound-inducible protein.

The present disclosure also pertains to a method of altering disease resistance of a plant, comprising providing a plant cell with a site-specific genome modification enzyme that cleaves a conserved region in one or more disease resistance loci and growing the plant from the plant cell. In one embodiment, the disease resistance locus comprises an NBS-LRR class of disease resistance genes.

The present disclosure further pertains to a method of providing a plant with improved disease resistance, comprising: (a) providing to one or more plant cells a site-specific genome modification enzyme that cleaves a target sequence in a disease resistance locus; (b) screening the one or more plant cells for asymmetric recombination between disease-resistance loci on homologous chromosomes to identify plant cells comprising a recombinant disease resistance locus; (c) testing plants obtained from the plant cells identified in step (b) and their progeny for improved disease resistance; and (d) selecting a plant with improved disease resistance. In addition, the present disclosure provides a plant, plant cell, plant seed or plant part generated by this method. In some embodiments, the plant has at least one recombinant disease-resistance locus. In some embodiments, the plant has at least one deletion in at least one of the disease-resistance loci. In one aspect, the plant has improved disease resistance compared with a plant with the parental allele of the disease resistance locus or a deletion of one or more genes in the disease-resistance locus. In one embodiment, the disease resistance locus comprises an NBS-LRR class of disease resistance genes.

Furthermore, the present disclosure provides a method of detecting homologous recombination between two parental chromosomes, comprising: a. identifying restriction nuclease sites flanking a targeted locus of interest on each of the two parental chromosomes; b. providing a PCR primer specific for the first parental chromosome and another PCR primer specific for the second parental chromosome; and c. using a probe designed to specifically recognize the unique junction of the 5′-flanking region of the locus of interest on the first chromosome and the 3′-flanking region of the locus of interest on the second chromosome to identify a PCR product which indicates the occurrence of recombination.

### BRIEF DESCRIPTION OF THE DRAWINGS

FIG. 1 illustrates induced asymmetric recombination between arrays of tandemly duplicated genes. In this illustration, the gene arrays have 1, 2, . . . , n tandemly duplicated genes and asymmetric recombination occurs between gene n on the first parental chromosome and gene 1 on the second parental chromosome resulting in the formation of a new array comprising a genes from both parent chromosomes (in this illustration 1, 2, 2, n) on a first recombinant chromosome and a single new gene (illustrated as 1/n) on a second recombinant chromosome. The lightening bolts indicate sites of genome modification.

FIG. 2 illustrates induced recombination between two genomic loci arranged in trans repulsion on the two parental chromosomes (left pair) and following genome modification the loci are in cis on the progeny chromosomes (right pair). The lightening bolt indicates a site of genome modification.

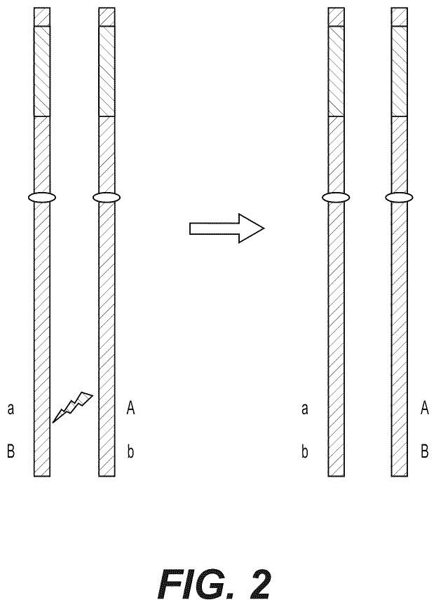

FIG. 3 illustrates a method of identifying recombination between two parental chromosomes by inverse PCR. Restriction nuclease sites (indicated by triangles) flanking a targeted genomic locus of interest are identified on each of the parental chromosomes. A PCR primer specific for the first parental chromosome (‘A’) is indicated by the black arrow. A PCR primer specific for the second parental chromosome (‘a’) is indicated by the gray arrow. An induced double-stranded break (indicated by the lightening bolt) promotes recombination between the parental chromosomes bringing both restriction endonuclease sites and primer binding sites onto the same recombinant chromosome. A TaqMan® probe specific for the unique junction of the 5′-flanking region of the targeted genomic locus of interest on the ‘A’ chromosome and the 3′-flanking region of the targeted genomic locus of interest on the ‘a’ chromosome is indicated by the bar with stars on each end. A PCR product is observed only in instances where recombination occurs.

FIG. 4 illustrates high-throughput genetic screening for transformants that underwent induced asymmetric recombination in homologous gene arrays. In this example, gene arrays containing 3 genes are present in each of the parental genomes. The assay identifies copy number variations in the R1 or BC1 generation following the induction of a double-stranded break (indicated by the lightening bolt) in target sequence located in a gene array. Genomic DNA isolated from R1 or BC1 plants is cleaved by a restriction endonuclease (triangle) that physically separates the genes. A TaqMan® probe designed to a conserved region of each gene in the array is used to detect new copy number variants.

FIG. 5 illustrates an assay strategy used to detect size shifts of PCR amplicons, where the size shift indicates the presence of recombinant paralogs.

FIGS. 6A-6B show a representative electrophoretic profile from capillary electrophoresis analysis of PCR amplicons using PCR primers EN1867 (SEQ ID NO:43) and EN1872 (SEQ ID NO:47), amplifying TS3 of the soybean Rps1 locus. Panel A shows three peaks corresponding to PCR amplicons of new length (marked as differential peaks with arrows) and a peak corresponding to a PCR amplicon of the size obtained with the control sample. Panel B shows a peak corresponding to the PCR amplicon generated with the control sample (Cas9 (−) Control).

FIGS. 7A-7B show a representative electrophoretic profile from capillary electrophoresis analysis of PCR amplicons using PCR primers EN1868 (SEQ ID NO:44) and EN1872 (SEQ ID NO:47), amplifying TS3 of the soybean Rps1 locus. Panel A shows two peaks corresponding to PCR amplicons of new length (marked as differential peaks with arrows). Panel B shows that no PCR amplicon was generated with this PCR primer pair with the control sample (Cas9 (−) Control).

FIGS. 8A-8B show a representative electrophoretic profile from capillary electrophoresis analysis of PCR amplicons using PCR primers EN1868 (SEQ ID NO:44) and EN1875 (SEQ ID NO:50), amplifying TS3 of the soybean Rps1 locus. Panel A shows one peak corresponding to a PCR amplicon of new length (marked as differential peak with arrow) and a peak corresponding to a PCR amplicon of the size obtained with the control sample. Panel B shows a peak corresponding to the PCR amplicon generated with the control sample (Cas9 (−) Control).

### DETAILED DESCRIPTION

Unless defined otherwise, all technical and scientific terms used herein have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of the present disclosure employs, unless otherwise indicated, conventional techniques of biochemistry, chemistry, molecular biology, microbiology, cell biology, genomics, plant breeding, and biotechnology, which are within the skill of the art. See Green and Sambrook, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)); Harlow and Lane, eds. (1988) ANTIBODIES, A LABORATORY MANUAL; ANIMAL CELL CULTURE (R. I. Freshney, ed. (1987)); RECOMBINANT PROTEIN PURIFICATION: PRINCIPLES AND METHODS, 18-1142-75, GE Healthcare Life Sciences; C. N. Stewart, A. Touraev, V. Citovsky, T. Tzfira eds. (2011) PLANT TRANSFORMATION TECHNOLOGIES (Wiley-Blackwell); and R. H. Smith (2013) PLANT TISSUE CULTURE. TECHNIQUES AND EXPERIMENTS (Academic Press, Inc.).

Any references cited herein are incorporated by reference in their entireties.

As used herein, the singular form “a,” “an,” and “the” include plural references unless the context clearly dictates otherwise. For example, the term “a compound” or “at least one compound” may include a plurality of compounds, including mixtures thereof.

As used herein, the term “about” indicates that a value includes the inherent variation of error for the method being employed to determine a value, or the variation that exists among experiments.

As used herein, the term “site-specific genome modification enzyme” refers to any enzyme that can modify a nucleotide sequence in a site-specific manner. In the present disclosure, site-specific genome modification enzymes include endonucleases, recombinases, transposases, helicases and any combination thereof.

As used herein, the term “recombination” refers to the process by which two DNA molecules exchange nucleotide sequences. In some embodiments, recombination occurs between two sets of parental chromosomes. In some embodiments, recombination occurs between two homologous chromosomes. In some embodiments, recombination occurs between non-homologous chromosomes. In some embodiments, recombination occurs between homoeologous chromosomes. In some embodiments, recombination results in the production of a new gene sequence, number of genes, arrangement of genes, allele or combination of alleles.

As used herein, the term “recombination event” refers to an instance of recombination between two DNA molecules.

As used herein, the term “homologous recombination” refers to the exchange of nucleotide sequences at a conserved region shared by two genomic loci. Homologous recombination includes symmetric homologous recombination and asymmetric homologous recombination. Asymmetric homologous recombination may also be referred to as unequal recombination.

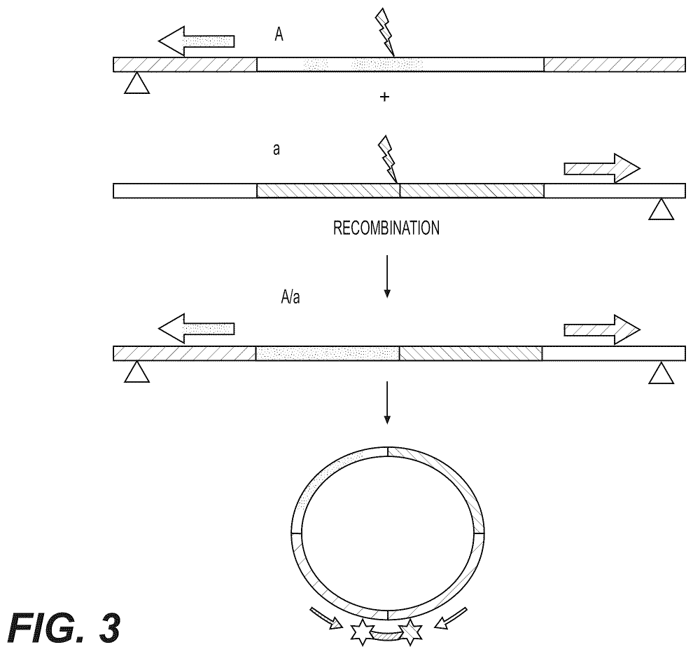

Many methods for detecting recombination are know in the art and include, but are not limited to, 1) phenotypic screening, 2) molecular marker technologies such as single nucleotide polymorphism—SNP analysis by TaqMan® or Illumina/Infinium technology, 3) Southern blot, and 4) sequencing. One example of a method for identifying recombination between two parental chromosomes by inverse PCR is illustrated in FIG. 3. In this method, restriction nuclease sites flanking a targeted gene of interest are identified on each of the two parental chromosomes. These restriction nuclease sites can be the same or different. A PCR primer specific for the first parental chromosome and another PCR primer specific for the second parental chromosome are designed. An induced double-stranded break promotes recombination between the two parental chromosomes bringing both restriction endonuclease sites and primer binding sites onto the same recombinant chromosome. A PCR product is observed only in instances where recombination occurs.

As used herein, the term “recombination rate” refers to the probability that a recombination event will occur between two genomic loci. The recombination rate may be influenced by a number of factors, including, but not limited to, the distance between two genomic loci, the chromosomal region (e.g., centromereic, telomereic) in which the loci occur, transcriptional activity, the presence of chromosomal inversions and other factors. Methods for measuring recombination include, but are not limited to, linkage analysis in mapping populations, and quantitative technologies such as quantitative PCR (qPCR) or droplet digital PCR (ddPCR), as described in the present disclosure.

As used herein, the term “genomic locus” refers to a specific location on a chromosome. A genomic locus may comprise a single nucleotide, a few nucleotides, a large number of nucleotides, a gene, a portion of a gene, a gene cluster, a multigene family or array of genes in a genomic region. In some embodiments, a genomic locus may comprise a tandemly duplicated array of genes. In some embodiments, a genomic locus may comprise a QTL. A genomic locus may be defined by a specific sequence, or a genomic locus may be defined by flanking markers. A genomic locus may also be defined by a linkage map.

As used herein, the term “target sequence” refers to a nucleotide sequence against which a site-specific genome modification enzyme binds and/or exerts cleavage, nickase, recombinase or transposase activity. A target sequence may be genic or non-genic.

As used herein, the term “gene” means a locatable region of genomic sequence corresponding to a unit of inheritance. A gene may include regulatory regions, such as promoters, enhancers, 5′-untranslated regions, intron regions, exon regions, 3′-untranslated regions, transcribed regions, and other functional sequence regions that may exist as native genes or transgenes in a plant or a mammalian genome. Depending upon the circumstances, the term “target gene” can refer to the full-length nucleotide sequence of a gene targeted for binding and/or cleavage or the nucleotide sequence of a portion of a gene targeted for binding and/or cleavage. A target gene can be an endogenous gene or a transgene.

As used herein, the term “event” refers to a genomic sequence resulting from molecular recombination of the cellular genomic DNA. The recombination includes homologous recombination, non-homologous recombination, cis-recombination, sister-chromatid exchange, multiple chromosome rearrangements, symmetric and asymmetric recombination. An event may occur in a genic sequence or the event may occur in an intergenic sequence. In some embodiments, an event may be a novel genomic sequence.

As used herein, an “elite line” is any line that has resulted from breeding and selection for superior agronomic performance.

As used herein, the term “inbred” means a line that has been bred for genetic homogeneity.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines. In some embodiments, a hybrid may be generated by crossing Triticum aestivum (wheat) with a plant selected from Aegilops ovate, Ae. biuncialis, Ae. triuncialis, Ae. quarrosa, Secale cereal, Triticum dicoccoides, Triticum dicoccum and Triticum durum. In some embodiments, a hybrid may be created by crossing Gossypium hirsutum (cotton) with a plant selected from G. sturtii, G. davidsonii, G. arboretum and G. raimondii.

As used herein, the term “marker” refers to a detectable characteristic that can be used to discriminate between organisms, genomes, chromosomes, genomic loci, genes or portions of genes. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

As used herein, the term “genetic marker” refers to a polymorphic nucleic acid sequence or nucleic acid feature that can be used to discriminate between nucleic acids. A “polymorphism” is a variation among individuals in sequence, particularly in DNA sequence, or feature, such as a transcriptional profile or methylation pattern. Useful polymorphisms may comprise, but are not limited to, one or more base changes, the insertion of one or more nucleotides or the deletion of one or more nucleotide, a single nucleotide polymorphism (SNP), a simple sequence repeat (SSR) and indels (insertions and deletions). A polymorphism may arise from random processes in nucleic acid replication, through mutagenesis, as a result of mobile genomic elements, from copy number variation and during the process of meiosis, such as unequal crossing over, genome duplication and chromosome breaks and fusions.

As used herein, the term “linkage” refers a phenomenon wherein genes on the same chromosome tend to segregate together more often than expected by chance if their transmission was independent. For example, if genomic locus A has genes “A” or “a” and genomic locus B has genes “B” or “b” and a cross between a first parental genome with AABB and a second parental genome with aabb will produce four possible gametes where the genes are segregated into AB, Ab, aB and ab. The null expectation is that there will be independent equal segregation into each of the four possible genotypes, i.e. with no linkage ¼ of the gametes will of each genotype. In this scenario, segregation of gametes into a genotypes differing from ¼ are attributed to linkage.

As used herein, “plant” refers to a whole plant, a plant cell, plant tissue or a plant seed. A cell or tissue culture derived from a plant can comprise any plant components or plant organs (e.g., leaves, stems, roots, etc.), plant tissues, seeds, plant cells, and/or progeny of the same. A progeny plant can be from any filial generation, e.g., F₁, F₂, F₃, F₄, F₅, F₆, F₇, etc. A plant cell is a biological cell of a plant, taken from a plant or derived through culture from a cell taken from a plant.

As used herein, “plant genome” refers to a nuclear genome, a mitochondrial genome, or a plastid (e.g., chloroplast) genome of a plant cell. In some embodiments, a plant genome may comprise a parental genome contributed by the male and a parental genome contributed by the female. In some embodiments, a plant genome may comprise only one parental genome.

The term “conserved region” refers to a contiguous polynucleotide sequence that is shared by two or more DNA sequences with at least 80%, at least 85%, at least 90%, at least 95%, at least 96%, at least 97%, at least 98%, at least 99%, or 100%, sequence identity. The conserved region can be at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, or more in length. As used herein, the term “identity” when used in relation to nucleic acids, describes the degree of similarity between two or more nucleotide sequences. The percentage of “sequence identity” between two sequences can be determined by comparing two optimally aligned sequences over a comparison window, such that the portion of the sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. An alignment of two or more sequences may be performed using any suitable computer program. For example, a widely used and accepted computer program for performing sequence alignments is CLUSTALW v1.6 (Thompson, et al. (1994) Nucl. Acids Res., 22: 4673-4680).

As used herein, the term “paralog” refers to genes related by duplication within a genome. In some embodiments paralogs are in the same genome. In some embodiments paralogs are in different genomes. In some embodiments paraologs are isofunctional. In some embodiments paralogs are heterofunctional.

As used in the term “homolog” refers to a gene related to a second gene by descent from a common ancestral DNA sequence. The term, homolog, may apply to the relationship between genes separated by the event of speciation (ortholog) or to the relationship between genes separated by the event of genetic duplication (paralog). In some embodiments homologs are in the same genome. In some embodiments homologs are in different genomes. In some embodiments homologs are isofunctional. In some embodiments homologs are heterofunctional.

As used in the term “homoeolog” refers a pair of genes that originated by speciation and were brought back together in the same genome by allopolyploidization.

As used in the term “homoeologous chromosome” refers to chromosomes of different species that share an ancestral origin.

As used herein, the term “tandem duplication” refers any occurrence of two identical sequences, one following the other, in a chromosome segment.

As used herein, the term “gene duplication” refers any duplications of a region of DNA that contains a gene.

A gene cluster is a group of two or more genes linked as neighbors on a chromosome. Genes in gene clusters often encode for similar polypeptides, or proteins, which collectively share a generalized function and are often located within a few thousand base pairs of each other.

A gene family is a set of several similar genes, formed by duplication of a single original gene. A gene family can comprise 2 or more genes, 3 or more genes, 4 or more genes, 5 or more genes, 6 or more genes, 7 or more genes, 8 or more genes, 9 or more genes, 10 or more genes, 15 or more genes, 20 or more genes, 25 or more genes, 30 or more genes, 35 or more genes, 40 or more genes, 45 or more genes, 50 or more genes, 55 or more genes, 60 or more genes, 65 or more genes, 70 or more genes, 75 or more genes, 80 or more genes, 90 or more genes, 100 or more genes, 150 or more genes, 200 or more genes, 250 or more genes, 300 or more genes, 350 or more genes, 400 or more genes, 450 or more genes, or 500 or more genes. In some embodiments a gene family may comprise an array of tandemly duplicated genes.

As used herein, the term “array of tandemly duplicated genes” refers to a gene cluster created by tandem duplication of chromosome segments containing one or more genes. In some embodiments, an array of tandemly duplicated genes can comprise 2 or more genes, 3 or more genes, 4 or more genes, 5 or more genes, 6 or more genes, 7 or more genes, 8 or more genes, 9 or more genes, 10 or more genes, 15 or more genes, 20 or more genes, 25 or more genes, 30 or more genes, 35 or more genes, 40 or more genes, 45 or more genes, 50 or more genes, 55 or more genes, 60 or more genes, 65 or more genes, 70 or more genes, 75 or more genes, 80 or more genes, 90 or more genes, 100 or more genes, 150 or more genes, 200 or more genes, 250 or more genes, 300 or more genes, 350 or more genes, 400 or more genes, 450 or more genes, or 500 or more genes. Examples of arrays of tandemly duplicated genes include, but are not limited to, genes that encode NBS-LRR or PRR disease resistance proteins, seed storage proteins, the cell wall component extension proteins, F-box proteins, ABC transporters, serine-threonine/tyrosine protein kinases, and ribosomal RNAs.

As used herein, the term “allele” refers to one of a number of alternative forms of the same gene or same genetic locus.

As used herein, the term “disease resistance locus” refers to a genomic region associated with disease or pathogen resistance in a plant. A disease resistance locus may comprise one or more genes, gene families, arrays of genes or QTLs encoding a protein or proteins that confer to a plant resistance to at least one disease or pathogen. In one embodiment, the disease resistance locus comprises one or more NBS-LRR disease resistance genes, also referred to as NB-LRR genes, R genes, LRR genes. In another embodiment, the disease resistance locus comprises one or more PRR disease resistance genes. The disease resistance locus may encompass a specific gene, cluster of genes, array of genes and/or gene family known to confer pathogen resistance, for example Rp1, or Rpp1, or Rps1. In another embodiment, the disease resistance locus comprises the Rgh1 locus. In another embodiment, the disease resistance locus comprises the Rgh4 locus. Alternatively, the disease resistance locus may encompass a genomic region but the actual gene/element composition conferring disease resistance is unknown.

As used herein, the term “immunoglobulin gene” refers to any gene encoding a region of an immunoglobulin heavy chain or light chain (e.g., the VH, VL, CH regions, the hinge region, the variable (V) segment, the diversity (D) segment, the joining (J) segment, or a portion thereof). The term immunoglobulin includes any immunoglobulin class, i.e., IgM, IgG, IgD, IgA and IgE, and any isotype.

As used herein, a “plant pathogen” is any organism or agent resulting in the infection of a plant or plant tissue. Common pathogens include viruses, bacteria, fungi, insects and nematodes.

As used herein, the term “quantitative trait locus” or “QTL” refers to a region of DNA that is associated with the differential expression of a phenotypic trait in at least one genetic background, e.g., in at least one breeding population. QTLs are closely linked to a gene or genes that underlie the trait in question. In some instances, the identity of the elements, genes, or set of genes underlying the trait are unknown. An example of a QTL associated with pathogen resistance is Rhg4.

As used herein, a cell or genome referred to as “haploid” has a single set of chromosomes and the reduced number of chromosomes (n) in the haploid plant is equal to that of the gamete.

As used herein, a cell or genome referred to as “diploid” has two sets of chromosomes and the chromosome number (2n) is equal to that of the zygote.

Tandem gene duplication plays a role in the accumulation of clusters of repeat genes, which in turn, contributes to the expansion of gene families. Examples of tandemly duplicated loci include those encoding NBS-LRR (nucleotide-binding site leucine-rich repeat) disease resistance proteins, pathogen recognition receptor (PRR) proteins, seed storage proteins, the cell wall component extension proteins, F-box proteins, ABC transporters, serine-threonine/tyrosine protein kinases, and ribosomal RNAs. Another example includes the Rhg1 locus which comprises at least three separate genes, and copy number variation (CNV) of this locus is associated with nematode resistance in soybean. Unequal recombination within an array of tademly duplicated genes or a multigene family can provide diversity. For example, unequal recombination can occur as often as once in every few thousand gametes in a single NBS-LRR gene cluster. However, for most gene clusters, unequal recombination occurs on orders of magnitude less frequently. From time to time, new plant pathogen biotypes emerge which require deployment of new resistant paralogs, genic variation, or altered CNV of a disease resistance locus. Currently, breeders are dependent on natural mechanisms of producing genetic diversity that rely on rare random mutation events for production of plants with resistance to the new plant pathogen biotypes.

One tandemly duplicated genomic locus of particular economic value encompasses the family of the NBS-LRR disease resistance genes. Based on the rate of unequal recombination in nature and the capabilities of current technologies to boost homologous recombination in plants, it is estimated that as high as ten percent of all transformants with custom endonucleases (for example, TALENs) would induce new NBS-LRR variants in a single gene cluster. These may encode for new resistance phenotypes against plant pathogens of agronomic importance. Following transformation, R0 plants or their progeny are phenotyped in bioassays to identify new biotic resistance traits.

In one aspect, the technology described in the present disclosure will facilitate the development of a high-throughput and inexpensive trait development platform to accelerate molecular variation in genomic loci, especially genomic loci comprising tandemly arrayed genomic regions. In one embodiment, the trait development is with genomic regions associated with biotic stress resistance. In another embodiment, the trait development is with genomic regions associated with abiotic stress resistance. In another embodiment, the trait development is with genomic regions associated with compositional quality. In another embodiment, the trait development is with genomic regions associated with stature. In another embodiment, the trait development is with genomic regions associated with maturity group. In another embodiment, the trait development is with genomic regions of cis-chromosome exchange. In another embodiment, the trait development is with sister chromosomal arm exchange. In another embodiment, the trait development is with chromosomal arm exchange between non-homologous chromosomes. In another embodiment, the trait development is with chromosomal arm exchange between homoeologous chromosomes. In yet another embodiment, the trait development is with multiple genomic region exchange across chromosomes within a single nucleus.

The compositions and methods described herein relate to the use of site-specific genome modification enzymes to generate novel alleles by stimulating recombination at selected target sequences in genomic loci. In some embodiments, the compositions and methods described herein may be used to generate novel variations in clusters of genes present in the plant or animal genomes. In some embodiments, the gene cluster may comprise an array of tandemly duplicated genes. In some embodiments, the gene cluster may comprise a gene family. In some embodiments, the gene cluster may be a disease resistance locus. In several embodiments, the compositions and methods described herein stimulate cis-recombination of a selected genomic locus from one germplasm line to a second germplasm line. In several embodiments, the compositions and methods described herein stimulate sister chromosome exchange. In several embodiments, the compositions and methods described herein stimulate exchange of genomic loci between homologous chromosomes. In several embodiments, the compositions and methods described herein stimulate exchange of genomic loci between non-homologous chromosomes. In several embodiments, the compositions and methods described herein stimulate exchange of genomic loci between homoeologous chromosomes. In several embodiments, the compositions and methods described herein stimulate unequal recombination between selected target sequences in a genomic locus. In several embodiments, the compositions and methods described herein stimulate multiple genomic exchange events within a single cell.

Several embodiments relate to a method of generating new alleles of a genomic locus, comprising contacting a cell with a site-specific genome modification enzyme that cleaves at least one target sequence in a genomic locus, thereby inducing recombination and selecting at least one progeny comprising a new allele of the genomic locus. In one aspect, the present disclosure provides method of generating new alleles of a genomic locus, comprising contacting a cell with a site-specific genome modification enzyme that cleaves at least one target sequence in an array of genes in a genomic locus, thereby inducing recombination with a second array of genes in the genomic locus and selecting at least one progeny comprising a new allele of the genomic locus. In some embodiments, the first and second arrays of genes are identical. In some embodiments, the first and second arrays of genes are homologous. In some embodiments, the first and second arrays of genes are homeologous. In some embodiments, the first and second arrays of genes are arrays of tandemly duplicated genes. In other embodiments, the first and second arrays of genes are heterologous. In some embodiments, two new alleles are generated as a result of the asymmetric recombination depending on the recombination site. In some embodiments, the asymmetric recombination results in deletion in at least one of the alleles.

In another aspect, the present disclosure provides a method of inducing recombination or increasing recombination rate between at least two target genomic DNA sequences in a plant cell or a mammal cell, comprising transforming the plant cell or the mammal cell with a site-specific genome modification enzyme that specifically induces a genome modification at least one target sequence inside a DNA region that is conserved between said at least two target DNA sequences.

In some embodiments, the cell is a plant cell. In a further embodiment, the plant cell is obtained from a plant selected from an inbred plant and a hybrid plant. In other embodiments, the cell is a mammalian cell. In a further embodiment, the mammalian cell is a human cell or, for example, a cell from a rodent (e.g., a mouse, rat, hamster, guinea pig), a rabbit, a pig, a non-human primate (e.g., monkey, chimpanzee, macaque) species, or any other mammals.

Several embodiments relate to a method of altering disease resistance of a plant or a mammal, comprising transforming the plant or the mammal with a site-specific genome modification enzyme that specifically cleaves a conserved region in one or more genomic loci. In some embodiments, the present disclosure provides a method of altering disease resistance of a plant, comprising providing a plant cell with a site-specific nuclease that cleaves a conserved region in one or more disease resistance loci and growing the plant from the plant cell.

The present disclosure further provides a method of providing a plant with improved disease resistance, comprising: (a) providing a site-specific genome modification enzyme that cleaves a target sequence in a genomic locus to one or more plant cells; (b) screening the one or more plant cells for a recombination event to identify plant cells comprising a recombinant genomic locus; (c) testing plants obtained from the plant cells identified in step (b) and their progeny for improved disease resistance; and (d) selecting a plant with improved disease resistance. In some embodiments, the targeted genomic locus comprises tandemly duplicated genomic regions. In some embodiments, the targeted genomic locus is comprised of identical arrays of tandemly duplicated sequence. In some embodiments, the targeted genomic locus is comprised of heterologous arrays of tandemly duplicated sequence. In some embodiments, the recombination is in an inbred plant. In some embodiments, the recombination is in a hybrid plant. In some embodiments, the recombination is on the same chromosome. In some embodiments the recombination is between two chromosomes. In some embodiments the chromosomes are homologous. In some embodiments the chromosomes are non-homologous. In some embodiments the chromosomes are homoeologous. In certain embodiments, the targeted genomic locus is associated with disease resistance. In certain embodiments the disease resistance locus includes genes encoding NBS-LRR genes. In certain embodiments the disease resistance locus is associated with resistance to soy cyst nematode, for example, the Rhg1 locus or the Rhg4 locus.

In some embodiments, the recombination of the targeted genomic locus is asymmetric recombination. In some embodiments, the recombination of the targeted genomic locus is symmetric recombination. In some embodiments, the site-specific genome modification enzyme increases the recombination rate at the targeted genomic locus by at least 2 fold, by at least 3 fold, by at least 4 fold, by at least 5 fold by at least 6 fold, by at least 7 fold, by at least 8 fold, by at least 9 fold, or at least 10 fold compared with the naturally occurring recombination rate of the targeted genomic locus.

### Site-Specific Genome Modification Enzymes

Several embodiments relate to promoting recombination by providing a site-specific genome modification enzyme. As used herein, the term “site-specific enzyme” refers to any enzyme that can modify a nucleotide sequence in a site-specific manner. In some embodiments, recombination is promoted by providing a single-strand break inducing agent. In some embodiments, recombination is promoted by providing a double-strand break inducing agent. In some embodiments, recombination is promoted by providing a strand separation inducing reagent. In one aspect, the site-specific genome modification enzyme is selected from an endonuclease, a recombinase, a transposase, a helicase or any combination thereof.

In one aspect, the endonuclease is selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof).

Non-limiting examples of recombinase include a tyrosine recombinase attached to a DNA recognition motif provided herein is selected from the group consisting of a Cre recombinase, a Gin recombinase a Flp recombinase, and a Tnp1 recombinase. In an aspect, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Site-specific genome modification enzymes, such as meganucleases, ZFNs, TALENs, Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof), RNA-guided nucleases (non-limiting examples of RNA-guided nucleases include the CRISPR associated nucleases, such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof) and engineered RNA-guided nucleases (RGNs), induce a genome modification such as a double-stranded DNA break (DSB) or single-strand DNA break at the target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of exogenous sequences by homologous recombination.

In one aspect of the present disclosure, site-specific genome modification enzymes are selected to induce a genome modification in one, a few, or many individual target sequences in genomic loci. In another aspect of the present disclosure, site-specific genome modification enzymes are selected to induce a genome modification in a region of the genome based upon the detection of tandem duplicated sequences identified through quantitative PCR methods. In yet another aspect of the present disclosure, site-specific genome modification enzymes are selected to induce a genome modification in a region of the genome known to be associated with copy number variation. The genome modifications stimulate DNA repair that can lead to alterations to the genomic loci by mechanisms such as removal of genomic regions, insertion/deletion mutations (indels) of a genomic region, indels within a genic region that disrupt the gene function, gene conversion, unequal recombination between non-parallel copies of tandemly duplicated arrays, recombination between homologous chromosomes, recombination between non-homologous chromosomes, recombination between homoeologous chromosomes, and other types of rearrangements. After exposure to the site-specific genome modification enzyme, the resulting alterations can be identified in various ways including phenotypic screens, sequencing, or molecular methods to identify novel variation. Because of the abundance of tandemly duplicated loci, site-specific genome modification enzymes could be designed that cut numerous copies, and expressed in plants to stimulate many different alterations. Site-specific genome modification enzymes may be expressed in plants such that one or more genome modifications occur within a genomic locus, and resulting progeny screened for molecular changes. Subsequently, the progeny with confirmed molecular rearrangements are screened phenotypically for novel phenotypes, such as improved yield, improved compositional quality, improved resistance to abiotic stress, altered stature, and resistance to specific plant pathogens.

### ZFNs

Zinc finger nucleases (ZFNs) are synthetic proteins characterized by an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger oo-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

### TALENs

Transcription activator-like effectors (TALEs) can be engineered to bind practically any DNA sequence. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs. The transcription activator-like effector (TALE) DNA binding domain can be fused to a functional domain, such as a recombinase, a nuclease, a transposase or a helicase, thus conferring sequence specificity to the functional domain.

Transcription activator-like effector nucleases (TALENs) are artificial restriction enzymes generated by fusing the transcription activator-like effector (TALE) DNA binding domain to a nuclease domain. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site. In some embodiments, the nuclease is selected from a group consisting of PvuII, MutH, TevI, FokI, AwI, MlyI, SbfI, SdaI, StsI, CleDORF, Clo051, and Pept071. When FokI is fused to a TALE domain each member of the TALEN pair binds to the DNA sites flanking a target site, the FokI monomers dimerize and cause a DSB at the target site.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain, and the number of bases between the two individual TALEN binding sites are parameters for achieving high levels of activity. PvuII, MutH, and TevI cleavage domains are useful alternatives to FokI and FokI variants for use with TALEs. PvuII functions as a highly specific cleavage domain when coupled to a TALE (see Yank et al. 2013. PLoS One. 8: e82539). MutH is capable of introducing strand-specific nicks in DNA (see Gabsalilow et al. 2013. Nucleic Acids Research. 41: e83). TevI introduces double-stranded breaks in DNA at targeted sites (see Beurdeley et al., 2013. Nature Communications. 4: 1762).

The relationship between amino acid sequence and DNA recognition of the TALE binding domain allows for designable proteins. Software programs such as DNA Works can be used to design TALE constructs. Other methods of designing TALE constructs are known to those of skill in the art. Doyle et al. (2012) TAL Effector-Nucleotide Targeter (TALE-NT) 2.0: tools for TAL effector design and target prediction. Nucleic Acids Res. 40(W1):W117-W122; Cermak (2011). Efficient design and assembly of custom TALEN and other TAL effector-based constructs for DNA targeting. Nucleic Acids Res. 39(12):e82.

### Meganucleases

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14-40 bp).

The engineering of meganucleases is more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

### Argonaute

The Argonaute protein family is a DNA-guided endonuclease. The Argonaute isolated from Natronobacterium gregoryi has been reported to be suitable for DNA-guided genome editing in human cells (Gao, et al. DNA-guided genome editing using the Natronobacterium gregoryi Argonaute. Nature Biotechnology 34:768-773 (2016). Argonaute endonucleases from other species have been identified, (non-limiting examples of Argonaute proteins include Thermus thermophilus Argonaute (TtAgo), Pyrococcus furiosus Argonaute (PfAgo), Natronobacterium gregoryi Argonaute (NgAgo), homologs thereof, or modified versions thereof). Each of these unique Argonaute endonucleases have associated a sequence encoding DNA guide.

### CRISPR

The CRISPR (clustered regularly interspaced short palindromic repeats)/Cas (CRISPR-associated) system is an alternative to synthetic proteins whose DNA-binding domains enable them to modify genomic DNA at specific sequences (e.g., ZFN and TALEN). Specificity of the CRISPR/Cas system is based on an RNA-guide that use complementary base pairing to recognize target DNA sequences. In some embodiments, the site-specific genome modification enzyme is a CRISPR/Cas system. In an aspect, a site-specific genome modification enzyme provided herein can comprise any RNA-guided Cas nuclease (non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof); and, optionally, the guide RNA necessary for targeting the respective nucleases.

CRISPR/Cas systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5′-NGG-3′ but less frequently NAG. Specificity is provided by the so-called “seed sequence” approximately 12 bases upstream of the PAM, which must match between the RNA and target DNA. Cpf1 acts in a similar manner to Cas9, but Cpf1 does not require a tracrRNA.

### Target Genes

The present disclosure can be applied to any genomic locus to generate genetic variation or recombination. In one embodiment, the genomic locus has tandemly duplicated copies of a genomic sequence. In some embodiments, the duplicated copies of genomic sequence are organized in gene clusters, for example as tandem arrays. Examples of such genomic regions include, but are not limited to genomic loci encompassing genes encoding NBS-LRR (nucleotide-binding site leucine-rich repeat) disease resistance proteins, pathogen recognition receptor (PRR) proteins, seed storage proteins, the cell wall component extension proteins, F-box proteins, ABC transporters, immunoglobulins, serine-threonine/tyrosine protein kinases, and ribosomal RNAs. In another aspect, the genomic region may be associated with a locus where copy number variation (CNV) is associated with resistance, such as resistance to soy cyst nematode (for example, Rhg1). In one aspect, the present disclosure is used to target and modify NBS-LRR disease resistance loci in order to generate new NBS-LRR variants that confer improved disease resistance to the plant. In another aspect, the present disclosure is used to target and modify loci with genes encoding immunoglobulins in order to generate new immunoglobulin variants in a mammal cell.

In one aspect, the present disclosure provides a method of generating a new array of tandemly duplicated genes, comprising contacting a cell with a site-specific genome modification enzyme that modifies the genome at least one target sequence in a first array of tandemly duplicated genes, thereby inducing recombination with a second array of tandemly duplicated genes and selecting at least one progeny comprising a new array of tandemly duplicated genes. In some embodiments, the new array of tandemly duplicated genes is produced by asymmetric recombination. In some embodiments, the target sequence is selected based on homology of surrounding sequence to a sequence in the second array of tandemly duplicated genes. In some embodiments, the target sequence and the sequence in the second array of tandemly duplicated genes are at different positions within the array of tandemly duplicated genes.

In some embodiments, the target sequence is in a genic region of the genome. In other embodiments, the target sequence is in an intergenic region of the genome.

In one embodiment, the target sequence for a site-specific genome modification enzyme is within a genic region of the selected genomic locus. In another embodiment, the target sequence for site-specific genome modification enzymes is in an inter-genic region of the selected genomic locus. In some embodiments, the selected genomic locus may comprise a QTL. In some embodiments, the selected genomic locus may comprise two or more tandemly arrayed gene units. In one embodiment the tandemly arrayed gene units are on the same chromosome. In another embodiment, the tandemly arrayed gene units are on different chromosomes. In some embodiments, the tandemly arrayed gene units are on homologous chromosomes. In some embodiments, the tandemly arrayed gene units are on non-homologous chromosomes. In some embodiments, the tandemly arrayed gene units are on homoeologous chromosomes. In another embodiment, the tandemly arrayed gene units are paralogs. In some embodiments, the tandemly arrayed gene units are homologs. In some embodiments, the tandemly arrayed gene units are homoeologs. In another embodiment, the tandemly arrayed gene units are different, more specifically one gene unit is not the same as another gene unit.

To promote asymetric recombination in a selected genomic locus or to promote recombination between to different genomic loci, or to promote recombination between homologous genomic loci, or to promote recombination between paralogous genomic loci, or to promote recombination between homoeologous genomic loci, or to promote recombination between genomic loci on homologous chromosomes, or to promote recombination between genomic loci on non-homologous chromosomes, or to promote recombination between genomic loci on homoeologous chromosomes, the of target sequences for the site-specific genome modification enzymes are selected in a way that the sequence of surrounding genomic regions are highly similar with a sequence in a genomic locus selected for recombination (the selected genomic locus may, for example, be in the same genomic locus as the target sequence, a different genomic locus as the target sequence, a different position with the genomic locus as the target sequence, in a paralogous genomic locus, in a homologous genomic locus, in a homoeologous genomic locus, on a homologous chromosome, on a homoeologous chromosome or on a paralogous chromosome) for at least 50 bp, at least 100 bp, at least 150 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1,000 bp. In some embodiments, the a sequence in the selected genomic locus and the sequence of genomic regions surrounding the target sequence may have at least 85% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity, over at least 50 bp, at least 100 bp, at least 150 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1,000 bp. In some embodiments, the selected genomic locus may contain a target sequence for the same site-specific genome modification enzyme or a different site-specific genome modification enzyme. This high level of homology in the genomic regions surrounding the target sequence facilitates recombination with the selected genomic locus. In some embodiments, the high level of homology in the genomic regions surrounding the target sequence facilitates asymmetric recombination. In some embodiments, due to polymorphisms within the selected genomic locus, the newly assembled genomic locus promotes the formation of novel copy number variants and/or novel genes. With genomic regions or loci included in the regions of homology, there is a higher probability for an increase in the number of variations created by random matches of homologous genome regions. If the target sequence is present in multiple genome regions, that may lead to development of new variants not only by unequal recombination, but also by deletion. The further apart two target sequences are from each other within the same genomic locus, the chances of deletion decrease, thus giving more opportunity for unequal recombination.

The plant immune system has two distinct, yet highly interconnected pathways to recognize and defend against pathogenic attacks. Some essential surface molecules of pathogenic cells, such as cell wall or flagellum components can be recognized by a variety of transmembrane proteins, mostly kinases. These pathogenic signals are collectively called pathogen-associated molecular patterns (PAMPs), while their receptors are called pathogen recognition receptors (PRRs). The defense mechanism against them is called PAMP-triggered immunity (PTI) that is regarded as the first line of molecular defense in plants. However, some pathogens have sophisticated mechanisms to get around this first line of defense and are able to actively transport effector molecules into the plant cells that are to re-program cell functions to the benefit of the pathogen. These effectors trigger the other major defense pathway, the effector-triggered immunity (ETI). Recognition proteins of ETI constitute a large superfamily with a conserved domain structure: they include a nucleotide-binding site (NBS) and a leucine-rich repeat (LRR) domain. These large, abundant, NBS-LRR proteins are involved in the detection of diverse pathogens, including bacteria, viruses, fungi, nematodes, insects and oomycetes. A major difference between PTI and ETI is that while the PAMP receptors are fairly conserved across large taxa, the NBS-LRR proteins are highly variable even within a species. This exceptional variability is a reflection of an intense arms race between plants and their pathogenic environment that keeps the NBS-LRR genes under strong diversifying selection pressure.

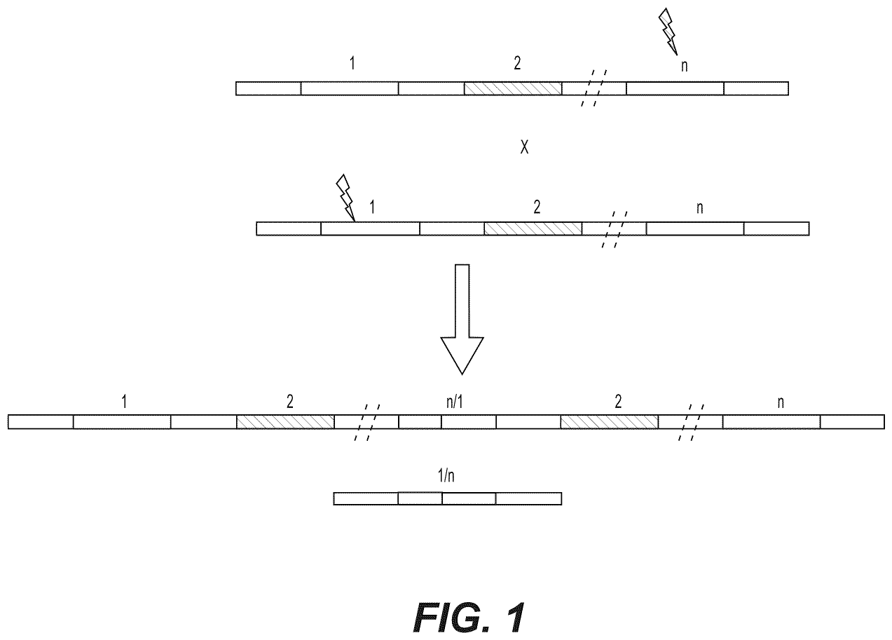

A typical plant species may have a few hundred NBS-LRR genes that are often organized in large clusters of tandem-duplicated gene units. For example, rice carries about 580 NBS-LRR genes. Most of them are distributed into tandem duplicated clusters. There are about 130 such clusters dispersed across the rice genome. The number of genes per cluster varies from two to eighteen, with a higher frequency at the lower end of the continuum. This genomic organization facilitates frequent asymmetric alignments between tandemly arrayed gene copies in meiocytes during meiotic cell division, in somatic cells during mitotic cell division, or in somatic cells during DNA repair. Asymmetric recombination may occur between identical alleles that can give rise to new alleles and allele-combinations of newly recombined gene units within the gene array as illustrated in FIG. 1. The unequal alignment also allows cross-over events between non-parallel gene copies that can give rise to new alleles and allele-combinations. Unequal (asymmetric) recombination as described above is widely viewed as the major mechanism contributing to the exceptional diversity of NBS-LRR loci. In nature, unequal recombination can occur as often as once in every few thousand gametes in a single NBS-LRR gene cluster. However, for most NBS-LRR gene clusters, they occur by orders of magnitude less frequently.

In one aspect, the present disclosure describes methods to accelerate the rate of novel allele development, particularly unequal recombination in NBS-LRR genomic loci using site-specific genome modification enzymes to induce genome modifications in the chromosomal DNA. These genome modifications can give rise to increased rates of asymmetric recombination, symmetric recombination, or production of indels, deletions, or inversions which in turn can give rise to new disease recognition specificities.

In some embodiments, the NBS-LRR disease resistance genes encode proteins that confer resistance to one or more diseases selected from various fungal rusts disease of maize or wheat; Fusarium diseases of various species, such as soy, maize and wheat; Goss's wilt, gray leaf spot and rust in maize, Asian soy rust; root-knot nematodes in soy and cotton; reniform nematode in cotton; and stem and leaf rust in wheat. In another embodiment, the NBS-LRR disease resistance genes encode proteins that confer resistance to one or more maize diseases selected from; Anthracnose Stalk Rot (Colletotrichum graminicola), Aspergillus Ear Rot (Aspergillus spp.), Common Rust of Corn (Puccinia sorghi), Diplodia Ear Rot (Diplodia frumenti, D. maydis), Diplodia Leaf Streak (Diplodia macrospora), Diplodia Stalk Rot (Diplodia frumenti, D. maydis), Eyespot (Aureobasidium zeae), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Head Smut (Sphacelotheca reiliana), Northern Corn Leaf Blight (Exserohilum turcicum), Pythium (Pythium spp.), Southern Leaf Blight (Cochliobolus heterostrophus). In another embodiment, the NBS-LRR disease resistance genes encode proteins that confer resistance to one or more soybean diseases selected from: Fusarium root rot (Fusarium spp.), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Stem Canker (Dioporthe phaseolorum). In another embodiment, the NBS-LRR disease resistance genes encode proteins that confer resistance to one or more cotton diseases selected from: Black root rot (Thielaviopsis basicola), Boll rot (Fusarium spp., Colletotrichum spp., Phytophthora spp., Rhizoctonia solani), Leaf spot (Alternaria spp., Cercospora gossypina, Rhizoctonia solani), Powdery mildew (Oidiopsis gossypii), Cotton rust (Puccinia spp., Phakopsora gossypii), Reniform nematode (Rotylenchulus reniformis), Root-knot nematode (Meloidogyne incognita), Alternaria leaf spot (Alternaria macrospora), Fusarium wilt (Fusarium oxysporurn f sp. vasinfectum), Verticillium wilt (Verticillium dahlia). In another embodiment, the NBS-LRR disease resistance genes encode proteins that confer resistance to one or more wheat diseases selected from: Fusarium head blight (Fusarium graminearum), Fusarium seedling blight (Fusarium spp., Septoria nodorum), Fusarium Leaf Blotch (Monographella nivalis), Leaf Rust (Puccinia triticina), Stem Rust (Puccinia graminis), Yellow Rust (Puccinia striiformis), Powdery Mildew (Blumeria graminis), Septoria Tritici Blotch (Septoria tritici), Septoria Nodorum Blotch (Septoria nodorum), Hessian Fly (Mayetiola destructor).

In some embodiments, the NBS-LRR disease resistance gene encode proteins that confer resistance to one or more diseases caused by viruses including, but are not limited pepper mottle virus, pepper mild mottle virus, cucumber mosaic virus, tomato yellow leaf curl virus, cucumber green mottle mosaic virus, potato virus Y, zucchini yellow mosaic virus, turnip mosaic virus, and rice stripe virus.

In some embodiments, a new allele of a disease resistance locus generated as described herein confers improved resistance to one or more diseases selected from various fungal rusts disease of maize or wheat; Fusarium diseases of various species, such as soy, maize and wheat; Goss's wilt, gray leaf spot and rust in maize, Asian soy rust; root-knot nematodes in soy and cotton; reniform nematode in cotton; and stem and leaf rust in wheat compared to a parent allele. In some embodiments, a new allele of a disease resistance locus generated as described herein confers improved resistance to one or more maize diseases selected from; Anthracnose Stalk Rot (Colletotrichum graminicola), Aspergillus Ear Rot (Aspergillus spp.), Common Rust of Corn (Puccinia sorghi), Diplodia Ear Rot (Diplodia frumenti, D. maydis), Diplodia Leaf Streak (Diplodia macrospora), Diplodia Stalk Rot (Diplodia frumenti, D. maydis), Eyespot (Aureobasidium zeae), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Head Smut (Sphacelotheca reiliana), Northern Corn Leaf Blight (Exserohilum turcicum), Pythium (Pythium spp.), Southern Leaf Blight (Cochliobolus heterostrophus) compared to a parent allele. In some embodiments, a new allele of a disease resistance locus generated as described herein confers improved resistance to one or more soybean diseases selected from: Fusarium root rot (Fusarium spp.), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Stem Canker (Diaporthe phaseolorum) compared to a parent allele. In some embodiments, a new allele of a disease resistance locus generated as described herein confers improved resistance to one or more cotton diseases selected from: Black root rot (Thielaviopsis basicola), Boll rot (Fusarium spp., Colletotrichum spp., Phytophthora spp., Rhizoctonia solani), Leaf spot (Alternaria spp., Cercospora gossypina, Rhizoctonia solani), Powdery mildew (Oidiopsis gossypii), Cotton rust (Puccinia spp., Phakopsora gossypii), Reniform nematode (Rotylenchulus reniformis), Root-knot nematode (Meloidogyne incognita), Alternaria leaf spot (Alternaria macrospora), Fusarium wilt (Fusarium oxysporurn f. sp. vasinfectum), Verticillium wilt (Verticillium dahlia) compared to a parent allele. In some embodiments, a new allele of a disease resistance locus generated as described herein confers improved resistance to one or more wheat diseases selected from: Fusarium head blight (Fusarium graminearum), Fusarium seedling blight (Fusarium spp., Septoria nodorum), Fusarium Leaf Blotch (Monographella nivalis), Leaf Rust (Puccinia triticina), Stem Rust (Puccinia graminis), Yellow Rust (Puccinia striiformis), Powdery Mildew (Blumeria graminis), Septoria Tritici Blotch (Septoria tritici), Septoria Nodorum Blotch (Septoria nodorum), Hessian Fly (Mayetiola destructor) compared to a parent allele.

In some embodiments, a new allele of a disease resistance locus generated as described herein confers improved resistance to one or more diseases caused by viruses including, but not limited to pepper mottle virus, pepper mild mottle virus, cucumber mosaic virus, tomato yellow leaf curl virus, cucumber green mottle mosaic virus, potato virus Y, zucchini yellow mosaic virus, turnip mosaic virus, and rice stripe virus compared to a parent allele.

In some embodiments, a new allele of a disease resistance locus generated as described herein confers improved resistance to one or more diseases selected from the group consisting of Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis) compared to a parent allele.

Additional selected viral or fungal disease inducing organisms can be selected from the list including: Acremonium, Alfamovirus, Allexivirus, Alternaria, Alternaria alternata, Ampelovirus, Aspergillus, Aspergillus oryzae, Aspergillus versicolor, Aureobasidium pullulans, Begomovirus, Bipolaris, Bipolaris sorokiniana (Helminthosporium blight), Bremia, Bremia lactucae, Bymovirus, Capillovirus, Carlavirus, Carmovirus, Caulimovirus, Cladosporium, Cladosporium herbarum, Closterovirus, Comovirus, Crinivirus, Cucumovirus, Cytorhabdovirus, Erisphe necator, Erysiphe, Fabavirus, Flexiviridae, Foveavirus, Furovirus, Geminivirus, Hordeivirus, Ilarvirus, Luteovirus, Maculavirus, Magnaporthe grisea (Gray Leaf Spot), Microdochium, Microdochium nivale (Pink Snow Mold), Nepovirus, Penicillium, Phoma, Phytoreovirus, Podosphaero macularis, Polerovirus, Pomovirus, Potexvirus, Potyvirus, Pyricularia, Rhizoctonia, Rhizoctonia oryzae (Rhizoctonia Sheath Spot), Sadwavirus, Sclerotinia, Septoria, Septoria apiicola, Spatherotheca fuliginea, Sphaerotheca, Stachybotrys, Stachybotrys chartarum, Taastrupvirus, Tenuivirus, Tobamovirus, Tobravirus, Tombusvirus, Tospovirus, Trichophyton, Trichophyton rubrum, Trichovirus.

In one aspect of the present disclosure, the plant is corn and the NBS-LRR is Rp1. In another aspect, the plant is soy and the NBS-LRR is Rpp1. In another aspect, the plant is soy and the NBS-LRR is Rps1. In yet another aspect, the plant is soy and the tandemly duplicated array is the Rhg1 locus conferring nematode resistance. In yet another aspect, the plant is soy and the disease resistance locus is the Rhg4 locus.

Soybean rust (SBR) is one of the most destructive diseases of soy. Several resistance loci have been identified in various germplasms against SBR, among which Rpp1 located on chromosome 18 is the most effective. The locus has been mapped to a 1 cM interval (Kim et al. 2012 Theor. Appl. Genet. 125:1339-1352). Physically, the corresponding chromosome segment harbors, among other genes, a few tandem repeat arrays of NBS-LRR genes, which are the most likely candidates for Rpp1. Unfortunately, new SBR biotypes that are virulent against Rpp1 have recently emerged that requires deployment of new resistant alleles of Rpp1, and those of other resistance loci, into commercial germplasms. Currently, breeders have to rely on natural mechanisms of genetic diversity at these loci, and then selection for new soybean varieties that were developed by these rare random mutations. Alternatively, transgenic approaches unrelated to the system described here may provide soybean events with resistance to SBR. Using the method proposed in this disclosure, the NBS-LRR gene families found in the Rpp1 region would be specifically targeted by site-specific genome modification enzymes to trigger fast development of new allelic configurations. These individual events will be genotyped by the high-throughput molecular method proposed below. Individuals comprising recombination events will be phenotypically analyzed for disease resistance.

Rp1 is a major resistance locus against the fungal rust disease of maize. It is located on chromosome 10 and is composed of tandem duplicated NBS-LRR gene units. Their copy number is highly variable among corn genotypes suggesting that meiotic rearrangements, described in this disclosure, occur frequently in the region. As a result, there are several haplotypes of Rp1, each of which is effective against a number of rust biotypes. On the other hand, given the typical high rate of development of new virulent races of pathogens, the current collection of Rp1 haplotypes widely used in commercial corn germplasms will probably be broken down soon by new pathogens. Targeted recombination in the Rp1 locus as described in this disclosure can develop new allele configurations at the pace or even faster than development of new virulent pathogens and thus breeders can keep rust under control by deployment of these new genes into commercial germplasms.

One of the most destructive root and stem rot diseases of soybean in the United States is caused by the Oomycete, pathogen Phytophthora sojae. Although soy cultivars exist with resistance to the causative P. sojae agent, there are documented shifts in susceptibility and virulence, indicating the need to continue to develop soy with diversity in the resistance loci in order to prevent wide crop loss.

Soy cyst nematode (SCN) is the most economically damaging pathogen of soybeans in the United States (Cook, 2012). The quantitative trait locus, Rhg1, in soybean was localized to chromosome 18 and found to confer resistance to SCN, though the molecular basis of the SCN resistance is unclear. The SCN resistance locus has been narrowed to a 31-kb region comprising three separate genes. It has been reported that that tandem repeats of 3 or 10 copies of the Rhg1 locus correlates to SCN resistance.

In one aspect of the present disclosure, site-specific genome modification enzymes, for example, endonucleases, designed for conserved regions of NBS-LRR gene clusters can significantly increase the recombination rate of the NBS-LRR gene clusters. Recombination frequencies were observed to increase about 1000-fold when double-strand breaks were introduced into plant genomes by custom endonucleases. Proportionally, one can assume that the rate of unequal recombinations can increase at a similar rate upon targeted nuclease activity. Assuming natural rates of 1/10,000 to 1/100,000 of unequal cross-overs, which was observed for multiple gene families, introduction of targeted double-strand breaks into new NBS-LRR alleles can result in a 1000-fold increase in frequency. Specifically, this may result in an increased frequency which occurs one per a few tens of transformants or a few hundreds of transformants. This would vastly expand the genotypic variation in NBS-LRR genes and thus their recognition specificities against various diseases.

In some embodiments, new NBS-LRR alleles can confer resistance against diseases that could not be achieved with conventional breeding methods. In some embodiments, new alleles can broaden the recognition capacities of existing disease resistance loci against wider ranges of disease causing biotypes.

In some embodiments, the target genes are immunoglobulin genes. In some embodiments, the target sequence is on a gene encoding part of a heavy chain (e.g., VH, CH1, CH2, CH3, CH4, the hinge region, or a portion thereof). In other embodiments, the target sequence is on a gene encoding part of a light chain (e.g., VL and CL, or a portion thereof). In some embodiments, the target sequence is on a gene encoding a variable (V) segment, a diversity (D) segment, a joining (J) segment, or a portion or any combination thereof. In some embodiments, the target sequence is in an intergenic region flanking the immunoglobulin genes. Human immunoglobulin genes are known to exist as tandem arrays of gene segments that undergo somatic recombination and hypermutation during B-lymphocyte development. These processes enable the generation of a vast repertoire of immunoglobulin molecules capable of recognizing and eliminating infectious agents.

### General Strategies for Generating New Arrays of Tandemly Duplicated Genes Using Site-Specific Genome Modification Enzymes

The following illustrates the general strategies for generating new alleles of genomic loci (such as disease resistance loci) using site-specific genome modification enzymes, such as site-specific endonucleases. The NBS-LRR disease resistance loci is a non-limiting example.

In one embodiment, a DNA sequence analysis is first carried out in a plant to identify short sequence motifs that are conserved by two or more targeted genomic loci. In some embodiments, the targeted genomic loci comprise tandem duplicated genes, gene families or gene clusters. In certain embodiments, the plant is selected from corn, soy, cotton, wheat, and canola. In certain embodiments, the targeted genomic loci comprise NBS-LRR genes. In some embodiments, the short sequence motifs are at least 50 bp, at least 100 bp, at least 150 bp, at least 200 bp, at least 250 bp, at least 300 bp, at least 350 bp, at least 400 bp, at least 450 bp, at least 500 bp, at least 600 bp, at least 700 bp, at least 800 bp, at least 900 bp, at least 1000 bp, at least 1100 bp, at least 1200 bp, at least 1300 bp, at least 1400 bp, at least 1500 bp, at least 1600 bp, at least 1700 bp, at least 1800 bp, at least 1900 bp, or at least 2000 bp in length. In some embodiments, the two or more targeted genomic loci share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of sequence identity in the identified short sequence motif. Site-specific genome modification enzymes may be designed to target the identified short sequence motif.

In some embodiments, similarity searches and subsequent design of site-specific genome modification enzymes focus on the LRR domains as opposed to the NBS domains of the NBS LRR locus. Since the LRR domains are directly involved in pathogen recognition, new recognition specificities are more likely to be generated if the LRR domains are rearranged internally and not just “swapped” as an intact domain to another NBS domain. Both corn and soy are assumed to carry several tens of conserved LRR signatures. Soybean also contains several islands of disease resistance that could be exploited, thus creating more genetic diversity in regions known to have associations with disease resistance. Cotton and wheat, being true allopolyploid species, have probably even more of such sequences.

Several custom site-specific genome modification enzymes are designed to target the identified short sequence motif. In some embodiments, the site-specific genome modification enzyme is an endonuclease. In some embodiments, the site-specific genome modification enzyme is a recombinase. In some embodiments, the site-specific genome modification enzyme is a transposase. In some embodiments, the site-specific genome modification enzyme is a helicase. In some embodiments, the nuclease is a transposase. In certain embodiments, the endonuclease is selected from a meganuclease, a ZFN, a TALEN, and a CRIPR/Cas system.

In some embodiments site-specific genome modification enzyme are tested for activity prior to transformation using a quantitative platform. One example of a quantitative platform that can be used in the present disclosure includes, but is not limited to, a protoplast-based oligo integration assay followed by digital PCR to detect the oligo integration. Efficacious site-specific genome modification enzyme are then advanced to large-scale stable transformation experiments in plants.

A method of detecting homologous recombination in the protoplast assay is illustrated in FIG. 3. In this method, target sequences, such as restriction nuclease sites, flanking a targeted gene of interest are identified on each of the two parental chromosomes. In some embodiments, the target sequences are the same. In other embodiments, the target sequences are different. In some embodiments, the target sequences are modified by the same site-specific genome modification enzyme. In other embodiments, the target sequences are modified by different site-specific genome modification enzymes. A PCR primer specific for the first parental chromosome and another PCR primer specific for the second parental chromosome are designed as indicated by the arrows. An induced double-stranded break promotes recombination between the two parental chromosomes brings both target sequences and primer binding sites onto the same recombinant chromosome. A TaqMan® probe is designed to specifically recognize the unique junction of the 5′-flanking region of the gene or array on the first chromosome and the 3′-flanking region of the gene or array on the second chromosome. A PCR product is observed only in instances where recombination occurs.

In certain embodiments, the plant is selected from: alfalfa, aneth, apple, apricot, artichoke, arugula, asparagus, avocado, banana, barley, beans, beet, blackberry, blueberry, brassica, broccoli, brussel sprouts, cabbage, canola, cantaloupe, carrot, cassava, cauliflower, celery, cherry, cilantro, citrus, clementine, coffee, corn, cotton, cucumber, Douglas fir, eggplant, endive, escarole, eucalyptus, fennel, figs, gourd, grape, grapefruit, honey dew, jicama, kiwifruit, lettuce, leeks, lemon, lime, Loblolly pine, mango, maize, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, raspberry, rice, rye, sorghum, Southern pine, soybean, spinach, squash, strawberry, sugarbeet, sugarcane, sunflower, sweet potato, sweetgum, sweet corn, tangerine, tea, tobacco, tomato, turf, a vine, watermelon, wheat, yams, and zucchini plants.

In certain embodiments, the plant is selected from: Alstroemeria (e.g., Alstoemeria brasiliensis), aster, azalea (e.g., Rhododendron sp.), begonias (e.g., Begonia sp.), bellflower, bouganvillea, cactus (e.g., Cactaceae schlumbergera truncata), camellia, carnation (e.g., Dianthus caryophyllus), chrysanthemums (e.g., Chrysanthemum sp.), clematis (e.g., Clematis sp.), cockscomb, columbine, cyclamen (e.g., Cyclamen sp.), daffodils (e.g., Narcissus sp.), false cypress, freesia (e.g., Freesia refracta), geraniums, gerberas, gladiolus (e.g., Gladiolus sp.), holly, hibiscus (e.g., Hibiscus rosasanensis), hydrangea (e.g., Macrophylla hydrangea), juniper, lilies (e.g., Lilium sp.), magnolia, miniroses, orchids (e.g., members of the family Orchidaceae), petunias (e.g., Petunia hybrida), poinsettia (e.g., Euphorbia pulcherima), primroses, rhododendron, Rosaceae, roses (e.g., Rosa sp.), snapdragons (e.g., Antirrhinum sp.), shrubs, trees such as forest (broad-leaved trees and evergreens, such as conifers) and tulips (e.g., Tulipa sp.).

In some embodiments, the plant is corn, maize, soybean, cotton, wheat, sorghum, or canola. In some embodiments, the plant is an inbred line or hybrid line.

In some embodiments, inbred lines are used for transformation. In other embodiments, hybrid lines are used for transformation. In hybrids, once genome modifications are made, not only unequal, but even regular homologous recombination would create new alleles at genomic loci, for example, heterozygous NBS-LRR gene clusters. Thus, where transformation is not strictly genotype-dependent transformation of hybrids with polymorphic disease resistance genes would produce the maximum frequency of novel variation. In cases where only certain lines are easily transformed, such as maize, near isogenic lines can be created that vary for disease resistance gene clusters but have similar transformation properties. Hybridization of such near isogenic lines will produce transformable lines that are diverse for disease resistance genes.

In some embodiments, the site-specific genome modification enzyme is stably transformed in the plant. In other embodiments, the site-specific genome modification enzyme is transiently transformed in the plant. In some embodiments, the site-specific genome modification enzyme is constitutively expressed in the plant. In other embodiment, the site-specific genome modification enzyme is expressed in the plant under the control of a tissue specific promoter. In other embodiment, the site-specific genome modification enzyme is expressed in the plant under the control of a regulatable promoter known in the art. In some embodiments, the regulatable promoter is a chemically inducible promoter.

In some embodiments, the site-specific genome modification enzyme is expressed in the plant under the control of a heat shock promoter, a tissue specific promoter, or a chemically inducible promoter.

The R0 plants or their selfed and/or backcrossed progenies are then phenotyped in bio-assays, for example, for new disease resistance phenotypes. High-throughput bio-assays for resistance against a panel of diseases (e.g. tests on leaf punches or very young seedlings) allow screening through thousands of plants in a fairly short period.

In some embodiments, a leaf disc assay is used for early, high-throughput screening. In some embodiments, leaf discs are collected and placed in a petri dish and inoculated with a pathogen using protocols known in the art. The dish is incubated at appropriate conditions to promote the pathogen growth and visual inspection is used to determine the resistance of the plant disc to damage by the pathogen.

In another embodiment, a leaf inoculation assay is used for high-throughput phenotypic screening, where a leaf is removed from the selected plant and is placed in a petri dish, inoculated with the pathogen, and the dish incubated at appropriate conditions to promote pathogen growth. Visual inspection is used to determine the resistance of the leaf to damage by the pathogen.

In yet another embodiment, individual plants are grown in pots in a greenhouse, inoculated with a pathogen and the phenotypic parameters of the plant growth are monitored. In some embodiments, the plant growth parameters which are tracked are plant height, canopy development, root architecture, fresh weight, stalk strength, and any other plant health parameter. Collection of the plant growth parameter data is done manually or with the systems of an automated greenhouse. In yet another embodiment, individual seeds from plants are evaluated in an agar medium or in a rolled filter assay to evaluate root and hypocoytl elongation (Chon et al, 2000). The agar or rolled paper towel may be imbibed with the test pathogen.

Several imaging systems optimized for field or green house conditions are used for quick and accurate phenotyping of massive amounts of plants in a fraction of the time that manual methods require. Examples include 2D or 3D imaging of whole plants or selected organs (e.g. leaves) using a variety of technologies, such as fluorescence, thermal or infrared cameras in automated or traditional greenhouse, or field settings.

Plants having passed the high-throughput leaf disc assay, leaf inoculation assay, root or hypocotyls elongation assay, and green house assay, are then screened for biotic stress in field trials. For this, seeds from the plants with the identified new locus are planted in the field in complete block design, the plants may or may not be inoculated with a pathogen and plant growth parameters are scored and yield is determined. Plants with resistance to biotic stress are used in a breeding program to introgress the new genotype into elite germplasm.

In some embodiments, constitutive expression of the site-specific genome modification enzyme is used because it may generate a steady level of stochastic alterations. In cases where it is possible to generate many transformants, constitutive expression may be used because each event will experience independent cutting and subsequent stochastic repair outcomes. Therefore, as an alternative to regulating the site-specific genome modification enzyme activity, many independent events can be screened. However, in other cases regulation of the timing of exposure of the genome to the custom nucleases (e.g. TALENs) may maximize the frequency of desired alterations.

Timing of site-specific genome modification enzyme activity should be done in a way that produces and allows recovery of many independent alterations. Correct timing of site-specific genome modification enzyme activity prevents “fixing” of one or a few alterations such as indels early in a cell lineage that then prevent subsequent cutting at that gene thereby minimizing the amount of variation that would be recovered at that location.

Certain tissue or cell types may have different relative levels of the various DNA repair pathways so that activation in a certain tissue or cell type may lead to a higher abundance of desired types of DNA repair and resulting alterations. For example, endonuclease activity at the beginning of meiosis may maximize the likelihood of recombination events between genes in clusters or between diverse clusters from different parents.

In some embodiments, regulation of site-specific genome modification enzyme activity is accomplished by delivery of the site-specific genome modification enzyme as gene cassettes with regulatable promoters. Such promoters will be known by those skilled in the art and include heat shock promoters, tissue specific promoters, chemically inducible promoters, or other environmentally regulated promoters. Additional methods to regulate site-specific genome modification enzyme activity include RNA stability, protein stability, protein localization, ligand inducible protein activation, conditional intein disruption and other methods.

Exposure, and therefore modification, of the genome to the site-specific genome modification enzyme at the beginning of meiosis may be optimal to stimulate meiotic recombination and unequal crossovers. Also, alterations at this stage would produce many independent gametes with different alterations that can be recovered in the next generation. Other options for timing of site-specific genome modification enzyme activity could be early in plant development such that each seed has a chance of producing different alterations or at a time when meristem branching is occurring such as during tassel development in corn.

In some embodiments, the conserved region in the target genomic locus is cleaved at the beginning of meiosis.

Besides unequal homologous recombination, several other types of mutations can also occur once double-strand breaks are created. For example, a mechanism known as synthesis-dependent strand annealing can lead to asymmetric gene conversions that, like asymmetric homologous recombination would also create new functional variants of the genes involved. Another mechanism, non-homologous end joining would create point mutations or short indels that would either cause amino-acid changes around the DSB, or render NBS-LRR genes non-functional by frame-shift mutations or by introducing a stop codon.

If DSBs occur concomitantly in two, tandemly arrayed NBS-LRR genes in the same chromosome, the fragment between the cuts being relatively short, may be eliminated as opposed to re-integrated into the genome. In this case, the chromosomal ends outside of the deleted fragment would be re-ligated by either single-strand annealing or non-homologous end joining. The net outcome of this scenario would be a new NBS-LRR allele and the loss of two or more large gene fragments. Gene conversion can also lead to the replacement of all or part of a gene with sequence from a related gene. The effect of this could be the generation of novel alleles by replacing portions of the coding regions from one gene with another or even the entire coding region such that regulatory portions of the first gene are now controlling a copy of the second gene.

Of these many possible DNA repair mechanisms, the ones creating new functional alleles, for example, unequal homologous recombination, are important for developing new disease resistant specificities. However, mutations rendering NBS-LRR genes non-functional can paradoxically also be very useful in developing new resistance phenotypes against certain types of pathogens. NBS-LRR genes trigger a pathway of programmed cell death (PCD) that kills the infected cells in plants, thus creating local lesions around the sites of pathogenic attack. There is a growing body of evidence that some necrotroph or hemibiotroph pathogens can corrupt this process to the benefit of the pathogen. Necrotroph or hemibiotroph pathogens capable of excreting host-specific toxins can stimulate NBS-LRR genes to an extent that PCD would destroy large segments of plant tissues instead of creating small lesions. These debilitated tissues can in turn be invaded by the pathogens. In all known cases of this type of pathogenesis, functional NBS-LRR genes caused disease susceptibility, while deletion or truncation of the causal NBS-LRR genes resulted in resistance. In some extreme cases, for example, “milo” disease for sorghum, such deletions caused total and irreversible immunity, so the disease ceased to be an economic problem. This mechanism of pathogenesis has been postulated for many important diseases including the sudden death syndrome (SDS) in soy caused by Fusarium species.

The present disclosure also provides a plant that is generated by the methods disclosed herein. In some embodiments, the site-specific genome modification enzyme modifies at least two genomic loci. In some embodiments, the site-specific genome modification enzyme modifies at least two of the disease-resistance genes or other tandemly duplicated genes. In one aspect, the plant has at least one recombinant genomic locus or other tandemly genomic locus. In one aspect, the plant has at least one deletion in one of the selected genomic loci. In one aspect, the plant has at least one recombinant disease-resistance gene or other tandemly duplicated gene. In one aspect, the plant has at least one deletion in one of the disease-resistance gene or other tandemly duplicated gene. In another aspect, the plant has improved disease resistance compared with a plant without the recombinant genomic locus or without the deletion in one of the selected genomic loci. In another aspect, the plant has improved disease resistance compared with a plant without a duplication in one of the selected genomic loci. In another aspect, the plant has improved disease resistance compared with a plant without the recombinant disease resistance gene or without the deletion in one of the disease-resistance gene. In one aspect, the plant does not have the site-specific genome modification enzyme in its genome.

In one aspect, the only exogenous gene transformed in the methods described in the present disclosure is the site-specific genome modification enzyme and any necessary selectable markers. Because the transgene will be segregated away after causing alterations to the genome, it will not be part of the final product. Events with multiple copies of the transgene and events with integrations into endogenous genes are normally discarded. However, in some embodiments they can be used as long as the site-specific genome modification enzyme is efficacious. This would allow running a high-throughput transformation and screening platform for rapid identification of new traits, such as, new disease resistance traits. This would also allow for the creation of improved disease resistance traits, wherein the improved disease resistance traits maintain robust resistance in the face of changing pathogen populations or wherein the improved traits provide resistance to a broader spectrum of pathogen races. In addition, it would save the cost of a large portion of event characterization.

The following Examples are presented for the purposes of illustration and should not be construed as limitations.

### EXAMPLES

### Example 1: Double-Strand Break Target Site Selection

To accelerate recombination in a selected genomic locus, comprehensive sequence analysis is done across a selected genomic locus in one or more selected germplasm lines to identify polymorphisms among both the genic and intergenic regions of the selected genomic locus. The sequencing is done by methods known to one skilled in the art. Non-limiting examples of sequencing methods include BAC clone sequencing, deep sequencing, “shot-gun” sequencing, random sequencing, direct sequencing, next-generation sequencing methods, etc. Bioinformatic tools are then used to identify specific target sequences for site-specific genome modification enzymes, such as a meganuclease, a zinc-finger nuclease, a zinc finger recombinase, an Argonaute, a TALEN, a TALER, an RNA-guided nuclease or CRISPR associated protein.

The target sequences are selected, in part, based on the sequence of the surrounding genomic region, such that the genomic regions surrounding the target sequence have a high level of homology over at least 100 bp with other regions within the selected genomic locus for. This high level of homology in genomic regions surrounding the target sequence facilitates unequal cross-over. Due to polymorphisms within the selected genomic locus, the newly assembled genomic locus promotes the formation of novel variants. Where the regions of homology within the genomic locus include multiple tandemly duplicated genes or multiple member of a multigene family, there is a higher probability for an increase in the number of variations created by random matches of homologous regions. If the target sequence is present in multiple places within the genomic locus, new variants may be formed not only by unequal recombination, but also by deletion. Where two target sequences are located at an increasing distance from each other, the chances of deletion between the target sequences decrease, thus giving more opportunity for unequal recombination.

In addition to using the sequencing data to select genome modification enzyme target sequences within the genomic locus of interest, the sequencing data may further be used to select germplasm to use for transformation. For example, sequencing data may be used to inform the selection of a specific elite germplasm or hybrid germplasm to use for transformation of the site-specific genome modification enzymes. Identification of germplasm for recombination at the selected genomic locus can be accomplished by re-sequenced across a set of individual plants representing varying germplasms and identifying polymorphisms among both the genic and intergenic regions of the selected genomic locus.

### Example 2: Protoplast Assay

A protoplast assay is used as a rapid assay for testing nuclease induced recombination within the selected genomic locus. Protoplasts are prepared from leaf mesophyll cells from the selected germplasm of corn plants identified as described in Example 1 and are heterozygous at the selected genomic locus. Similarly, protoplasts are prepared from cotyledon of soy plants from the selected germplasm identified as described in Example 1 and are heterozygous at the selected genomic locus. For the protoplast assay, individual plants which have polymorphism on the flanking sides of the selected genomic locus are chosen to facilitate screening by one or more PCR assay configurations.

Site-specific genome modification enzymes chosen and/or designed for the specific target sequences(s) identified as described in Example 1 are cloned into expression cassettes with plant-specific expression elements. Expression elements include enhancers, promoters, introns, 5′-untranslated leader sequences (5′-UTR), and 3′-untranslated polyadenylation sequences (3′UTR). The expression cassettes are further codon optimized using methods known in the art, for example the expression cassettes for use in corn use monocot codon optimization, and the expression cassettes for use in soybean use dicot codon optimization. The expression cassettes are incorporated into transformation vectors useful for protoplast assays.

The protoplasts are transformed using standard protoplast transformation protocols (for example, electroporation or polyethylene glycol (PEG)) with a transformation vector containing at least one expression cassette encoding a site-specific genome modification enzyme. In one embodiment, the site-specific genome modification enzyme is a CRISPR/Cas9 nuclease with at least one guide RNA targeting one or more of the selected target sequences in the genomic locus. In another embodiment, the site-specific genome modification enzyme is a TALEN. In another embodiment, the site-specific genome modification enzyme is a CRISPR associated protein linked to a recombinase. In another embodiment, the site-specific genome modification enzyme is an Argonaute. In another embodiment, the site-specific genome modification enzyme is a recombinase. In yet another embodiment, the site-specific genome modification enzyme is a meganuclease. In another embodiment, the site-specific genome modification enzyme is a TALE recombinase.

The transformation vector containing the site-specific genome modification enzyme expression cassette is transformed into the protoplasts. The design of the protoplast assay is such that the site-specific genome modification enzyme introduces a genome modification, for example, a double strand break (DSB), single-strand break, a transposase-mediated DNA exchange reaction or a recombinase-mediated DNA exchange reaction, in the selected genomic locus to promote recombination. The transformed protoplasts are harvested 48 to 72 hours later and genomic DNA is extracted. In one PCR assay configuration, restriction nuclease sites are chosen in the flanking regions of the genes in a way that when the fragments, following digestion and self-ligation, will create a unique template for a TaqMan® probe. This unique template is not present by self-ligation of either of the parental chromosomes, but is specific for the template generated by self-ligation of the recombinant chromosome (see FIG. 3). This TaqMan® probe is used for quantification of recombinant chromosomes. This assay does not require polymorphism in the genomic locus of interest between the parents giving rise to the protoplast germplasm, only in their wider genetic environment.

A method of detecting recombination in the protoplast assay is illustrated in FIG. 3. In the figure, restriction nuclease sites are represented by the triangles below the 5′-flank of the “A” paralog, the 3′-flank of the ‘a’ paralog, and at both the 5′-flank and 3′-flank of the new recombined “A/a” paralog. Following restriction nuclease digestion of the genomic DNA, the genomic fragments are allowed to self-ligate to from circular molecules. When homologous recombination occurs at the locus, then the 5′-flank of the “A” paralog and the 3′-flank of the “a” paralog are joined. These rare recombination events are detected using inverse PCR with drop digital PCR (ddPCR) technology. Specifically, one PCR primer is designed to be unique to the 5′-flank of the “A” paralog, the second PCR primer is designed to be unique to the 3′-flank of the “a” paralog, and the PCR probe is designed to be unique to the junction created by self-ligation of the 5′-flank of the “A” paralog and the 3′-flank of the “a” paralog. A PCR product is observed only in instances where recombination occurs.

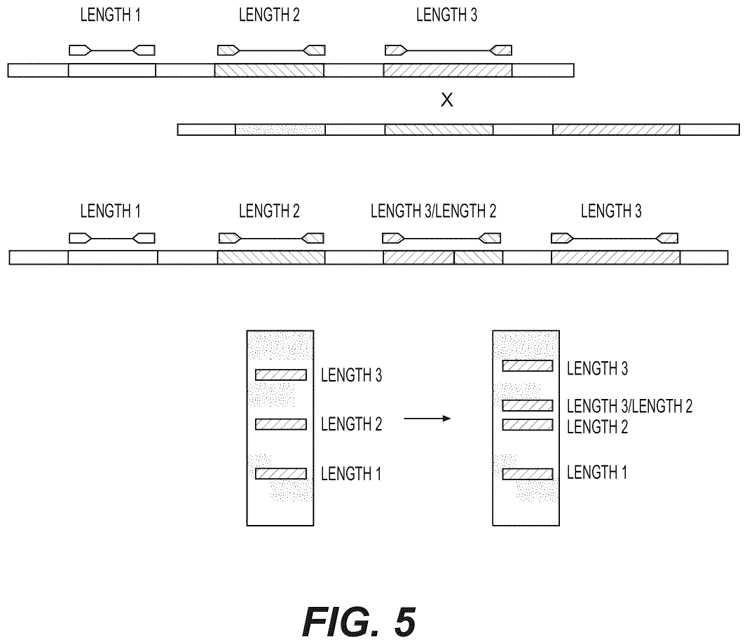

Another PCR assay configuration to detect homologous recombination is presented in FIG. 5. In this assay, PCR primer combinations are selected to amplify one or more individual genomic regions within the selected genomic locus such that the individual PCR amplicons differ in length. Upon recombination, PCR amplicons of a new size are generated with the same primer pairs. The PCR amplicons are resolved by either gel electrophoresis or by capillary electrophoresis.

### Example 3: Protoplast Assay to Detect Recombination in Soybean Rps1 Locus

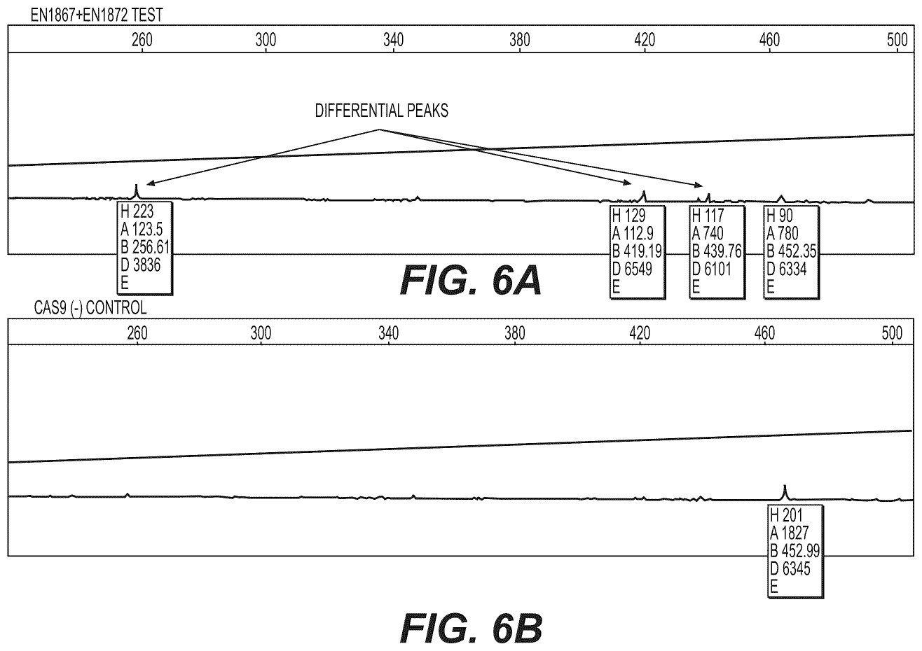

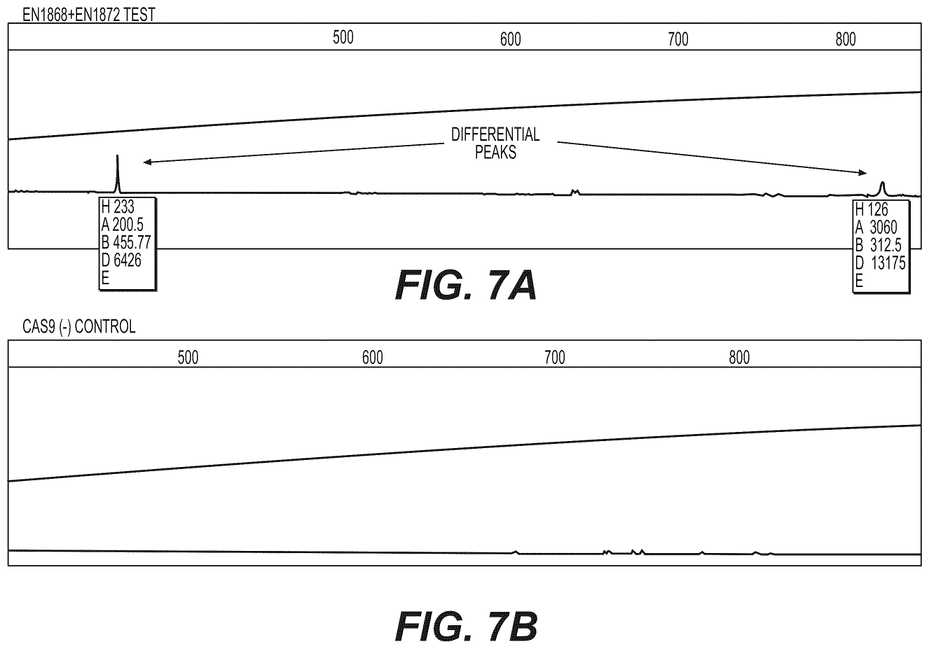

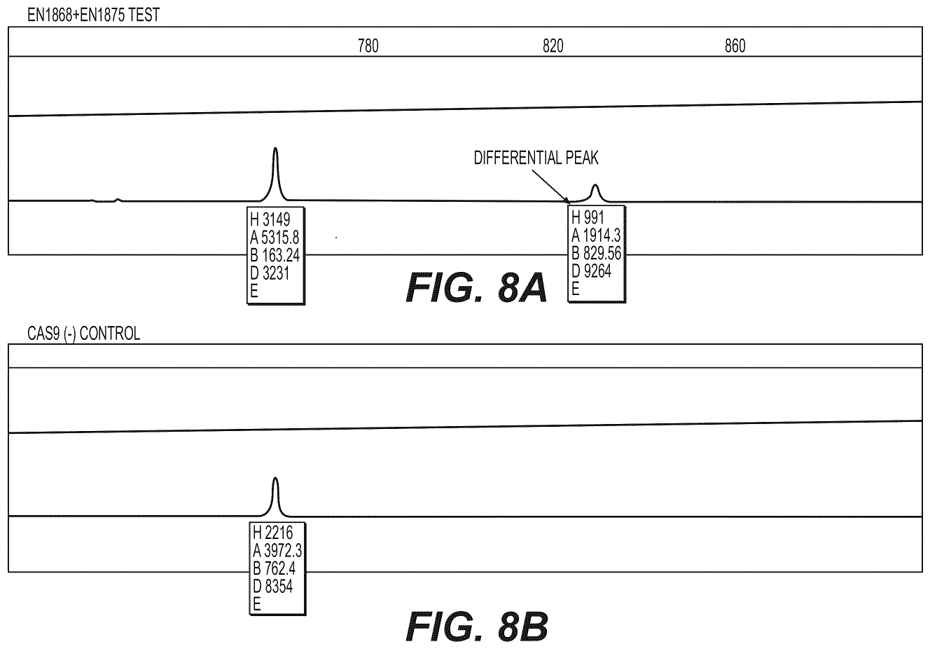

Cotyledon protoplasts of the soy germplasm A3555 were co-transformed with a vector containing an expression cassette for a soy codon-optimized Cas9 (SEQ ID NO:77) and one or two single guide RNA (sgRNA) constructs driven by a soy U6i (SEQ ID NO:80) or U6c (SEQ ID NO:81) promoter. The sgRNA were designed to one of the 6 identified RpsI target sequences (TS) represented by SEQ ID NO:21 through SEQ ID NO:32. For each of the six RpsI target sequences, there were two variants differing by 1 nucleotide (see Table 4). A Renilla luciferase construct was used as a transformation control. Following a two-day incubation period, total genomic DNA was isolated from the transformed protoplasts. Recombinant Rps1 paralogs were identified using a PCR assay as described in Example 1 that utilizes sequence length-polymorphism among individual Rps1 paralogs. While Rps1 paralogs are fairly conserved, there is significant length polymorphism among them due to multiple short indels distributed along their lengths. Therefore, PCR amplicons between two conserved primers will vary in size over a broad range. For example, the PCR amplicons generated between two highly conserved regions in 21 separate Rps1 paralogs of the soybean Williams 82 (W82) reference soy genome each have a unique PCR amplicon length. To assay the DNA extracted from the soybean protoplasts of this Example, 35 PCR primers (SEQ ID NO:33-67) were used in PCR amplification of the DNA extracted from the transformed soy protoplasts. These PCR primers represented 163 different primer pairs, with 73 unique primer combinations (Unique Primer Combo) as detailed in Table 1. Using capillary electrophoresis of the PCR reactions, 24 novel amplicons were identified. Examples of sequence length variation results are illustrated by the electrophoretic profiles from capillary electrophoresis analysis of PCR amplicons presented in FIGS. 6, 7, and 8. The sequence length variants are expected to represent recombinant RpsI loci. Novel RpsI variants are identified by sequence analysis of the PCR amplicons.

TABLE 1
Rps1 protoplast assay primer matrix.

| | | Primer 1 | | Primer 1 | | Unique |
|---|---|---|---|---|---|---|
| PCR | | SEQ | | SEQ ID | Target | Primer |
| Mix # | Primer 1 | ID NO | Primer 2 | NO | Sequence | Combo |
| 1 | EN1890 | 61 | EN1858 | 35 | TS1 | 1 |
| 2 | EN1892 | 62 | EN1858 | 35 | TS1 | 2 |
| 3 | EN1893 | 63 | EN1858 | 35 | TS1 | 3 |
| 4 | EN1894 | 64 | EN1858 | 35 | TS1 | 4 |
| 5 | EN1896 | 65 | EN1858 | 35 | TS1 | 5 |
| 6 | EN1897 | 66 | EN1858 | 35 | TS1 | 6 |
| 7 | EN1890 | 61 | EN1859 | 36 | TS1 | 7 |
| 8 | EN1892 | 62 | EN1859 | 36 | TS1 | 8 |
| 9 | EN1893 | 63 | EN1859 | 36 | TS1 | 9 |
| 10 | EN1894 | 64 | EN1859 | 36 | TS1 | 10 |
| 11 | EN1896 | 65 | EN1859 | 36 | TS1 | 11 |
| 12 | EN1897 | 66 | EN1859 | 36 | TS1 | 12 |
| 13 | EN1898 | 67 | EN1859 | 36 | TS1 | 13 |
| 14 | EN1890 | 61 | EN1860 | 37 | TS1 | 14 |
| 15 | EN1892 | 62 | EN1860 | 37 | TS1 | 15 |
| 16 | EN1893 | 63 | EN1860 | 37 | TS1 | 16 |
| 17 | EN1894 | 64 | EN1860 | 37 | TS1 | 17 |
| 18 | EN1896 | 65 | EN1860 | 37 | TS1 | 18 |
| 19 | EN1897 | 66 | EN1860 | 37 | TS1 | 19 |
| 20 | EN1898 | 67 | EN1860 | 37 | TS1 | 20 |
| 21 | EN1856 | 33 | EN1864 | 41 | TS2 | 21 |
| 22 | EN1857 | 34 | EN1864 | 41 | TS2 | 22 |
| 23 | EN1861 | 38 | EN1864 | 41 | TS2 | 23 |
| 24 | EN1862 | 39 | EN1864 | 41 | TS2 | 24 |
| 25 | EN1863 | 40 | EN1864 | 41 | TS2 | 25 |
| 26 | EN1866 | 42 | EN1871 | 46 | TS3 | 29 |
| 27 | EN1866 | 42 | EN1872 | 47 | TS3 | 30 |
| 28 | EN1866 | 42 | EN1873 | 48 | TS3 | 31 |
| 29 | EN1866 | 42 | EN1874 | 49 | TS3 | 32 |
| 30 | EN1866 | 42 | EN1875 | 50 | TS3 | 33 |
| 31 | EN1866 | 42 | EN1876 | 51 | TS3 | 34 |
| 32 | EN1866 | 42 | EN1877 | 52 | TS3 | 35 |
| 33 | EN1866 | 42 | EN1878 | 53 | TS3 | 36 |
| 34 | EN1866 | 42 | EN1879 | 54 | TS3 | 37 |
| 35 | EN1866 | 42 | EN1880 | 55 | TS3 | 38 |
| 36 | EN1866 | 42 | EN1881 | 56 | TS3 | 39 |
| 37 | EN1866 | 42 | EN1882 | 57 | TS3 | 40 |
| 38 | EN1866 | 42 | EN1883 | 58 | TS3 | 41 |
| 39 | EN1866 | 42 | EN1884 | 59 | TS3 | 42 |
| 40 | EN1866 | 42 | EN1885 | 60 | TS3 | 43 |
| 41 | EN1867 | 43 | EN1871 | 46 | TS3 | 44 |
| 42 | EN1867 | 43 | EN1872 | 47 | TS3 | 45 |
| 43 | EN1867 | 43 | EN1873 | 48 | TS3 | 46 |
| 44 | EN1867 | 43 | EN1874 | 49 | TS3 | 47 |
| 45 | EN1867 | 43 | EN1875 | 50 | TS3 | 48 |
| 46 | EN1867 | 43 | EN1876 | 51 | TS3 | 49 |
| 47 | EN1867 | 43 | EN1877 | 52 | TS3 | 50 |
| 48 | EN1867 | 43 | EN1878 | 53 | TS3 | 51 |
| 49 | EN1867 | 43 | EN1879 | 54 | TS3 | 52 |
| 50 | EN1867 | 43 | EN1880 | 55 | TS3 | 53 |
| 51 | EN1867 | 43 | EN1881 | 56 | TS3 | 54 |
| 52 | EN1867 | 43 | EN1882 | 57 | TS3 | 55 |
| 53 | EN1867 | 43 | EN1883 | 58 | TS3 | 56 |
| 54 | EN1867 | 43 | EN1884 | 59 | TS3 | 57 |
| 55 | EN1867 | 43 | EN1885 | 60 | TS3 | 58 |
| 56 | EN1868 | 44 | EN1871 | 46 | TS3 | 59 |
| 57 | EN1868 | 44 | EN1872 | 47 | TS3 | 60 |
| 58 | EN1868 | 44 | EN1873 | 48 | TS3 | 61 |
| 59 | EN1868 | 44 | EN1874 | 49 | TS3 | 62 |
| 60 | EN1868 | 44 | EN1875 | 50 | TS3 | 63 |
| 61 | EN1868 | 44 | EN1876 | 51 | TS3 | 64 |
| 62 | EN1868 | 44 | EN1877 | 52 | TS3 | 65 |
| 63 | EN1868 | 44 | EN1878 | 53 | TS3 | 66 |
| 64 | EN1868 | 44 | EN1879 | 54 | TS3 | 67 |
| 65 | EN1868 | 44 | EN1880 | 55 | TS3 | 68 |
| 66 | EN1868 | 44 | EN1881 | 56 | TS3 | 69 |
| 67 | EN1868 | 44 | EN1882 | 57 | TS3 | 70 |
| 68 | EN1868 | 44 | EN1883 | 58 | TS3 | 71 |
| 69 | EN1868 | 44 | EN1884 | 59 | TS3 | 72 |
| 70 | EN1868 | 44 | EN1885 | 60 | TS3 | 72 |
| 71 | EN1866 | 42 | EN1871 | 46 | TS4 | 29 |
| 72 | EN1866 | 42 | EN1872 | 47 | TS4 | 30 |
| 73 | EN1866 | 42 | EN1873 | 48 | TS4 | 31 |
| 74 | EN1866 | 42 | EN1874 | 49 | TS4 | 32 |
| 75 | EN1866 | 42 | EN1875 | 50 | TS4 | 33 |
| 76 | EN1866 | 42 | EN1876 | 51 | TS4 | 34 |
| 77 | EN1866 | 42 | EN1877 | 52 | TS4 | 35 |
| 78 | EN1866 | 42 | EN1878 | 53 | TS4 | 36 |
| 79 | EN1866 | 42 | EN1879 | 54 | TS4 | 37 |
| 80 | EN1866 | 42 | EN1880 | 55 | TS4 | 38 |
| 81 | EN1866 | 42 | EN1881 | 56 | TS4 | 39 |
| 82 | EN1866 | 42 | EN1882 | 57 | TS4 | 40 |
| 83 | EN1866 | 42 | EN1883 | 58 | TS4 | 41 |
| 84 | EN1866 | 42 | EN1884 | 59 | TS4 | 42 |
| 85 | EN1866 | 42 | EN1885 | 60 | TS4 | 43 |
| 86 | EN1867 | 43 | EN1871 | 46 | TS4 | 44 |
| 87 | EN1867 | 43 | EN1872 | 47 | TS4 | 45 |
| 88 | EN1867 | 43 | EN1873 | 48 | TS4 | 46 |
| 89 | EN1867 | 43 | EN1874 | 49 | TS4 | 47 |
| 90 | EN1867 | 43 | EN1875 | 50 | TS4 | 48 |
| 91 | EN1867 | 43 | EN1876 | 51 | TS4 | 49 |
| 92 | EN1867 | 43 | EN1877 | 52 | TS4 | 50 |
| 93 | EN1867 | 43 | EN1878 | 53 | TS4 | 51 |
| 94 | EN1867 | 43 | EN1879 | 54 | TS4 | 52 |
| 95 | EN1867 | 43 | EN1880 | 55 | TS4 | 53 |
| 96 | EN1867 | 43 | EN1881 | 56 | TS4 | 54 |
| 97 | EN1867 | 43 | EN1882 | 57 | TS4 | 55 |
| 98 | EN1867 | 43 | EN1883 | 58 | TS4 | 56 |
| 99 | EN1867 | 43 | EN1884 | 59 | TS4 | 57 |
| 100 | EN1867 | 43 | EN1885 | 60 | TS4 | 58 |
| 101 | EN1868 | 44 | EN1871 | 46 | TS4 | 59 |
| 102 | EN1868 | 44 | EN1872 | 47 | TS4 | 60 |
| 103 | EN1868 | 44 | EN1873 | 48 | TS4 | 61 |
| 104 | EN1868 | 44 | EN1874 | 49 | TS4 | 62 |
| 105 | EN1868 | 44 | EN1875 | 50 | TS4 | 63 |
| 106 | EN1868 | 44 | EN1876 | 51 | TS4 | 64 |
| 107 | EN1868 | 44 | EN1877 | 52 | TS4 | 65 |
| 108 | EN1868 | 44 | EN1878 | 53 | TS4 | 66 |
| 109 | EN1868 | 44 | EN1879 | 54 | TS4 | 67 |
| 110 | EN1868 | 44 | EN1880 | 55 | TS4 | 68 |
| 111 | EN1868 | 44 | EN1881 | 56 | TS4 | 69 |
| 112 | EN1868 | 44 | EN1882 | 57 | TS4 | 70 |
| 113 | EN1868 | 44 | EN1883 | 58 | TS4 | 71 |
| 114 | EN1868 | 44 | EN1884 | 59 | TS4 | 72 |
| 115 | EN1868 | 44 | EN1885 | 60 | TS4 | 73 |
| 116 | EN1866 | 42 | EN1871 | 46 | TS5 | 29 |
| 117 | EN1866 | 42 | EN1872 | 47 | TS5 | 30 |
| 118 | EN1866 | 42 | EN1873 | 48 | TS5 | 31 |
| 119 | EN1866 | 42 | EN1874 | 49 | TS5 | 32 |
| 120 | EN1866 | 42 | EN1875 | 50 | TS5 | 33 |
| 121 | EN1866 | 42 | EN1876 | 51 | TS5 | 34 |
| 122 | EN1866 | 42 | EN1877 | 52 | TS5 | 35 |
| 123 | EN1866 | 42 | EN1878 | 53 | TS5 | 36 |
| 124 | EN1866 | 42 | EN1879 | 54 | TS5 | 37 |
| 125 | EN1866 | 42 | EN1880 | 55 | TS5 | 38 |
| 126 | EN1866 | 42 | EN1881 | 56 | TS5 | 39 |
| 127 | EN1866 | 42 | EN1882 | 57 | TS5 | 40 |
| 128 | EN1866 | 42 | EN1883 | 58 | TS5 | 41 |
| 129 | EN1866 | 42 | EN1884 | 59 | TS5 | 42 |
| 130 | EN1866 | 42 | EN1885 | 60 | TS5 | 43 |
| 131 | EN1867 | 43 | EN1871 | 46 | TS5 | 44 |
| 132 | EN1867 | 43 | EN1872 | 47 | TS5 | 45 |
| 133 | EN1867 | 43 | EN1873 | 48 | TS5 | 46 |
| 134 | EN1867 | 43 | EN1874 | 49 | TS5 | 47 |
| 135 | EN1867 | 43 | EN1875 | 50 | TS5 | 48 |
| 136 | EN1867 | 43 | EN1876 | 51 | TS5 | 49 |
| 137 | EN1867 | 43 | EN1877 | 52 | TS5 | 50 |
| 138 | EN1867 | 43 | EN1878 | 53 | TS5 | 51 |
| 139 | EN1867 | 43 | EN1879 | 54 | TS5 | 52 |
| 140 | EN1867 | 43 | EN1880 | 55 | TS5 | 53 |
| 141 | EN1867 | 43 | EN1881 | 56 | TS5 | 54 |
| 142 | EN1867 | 43 | EN1882 | 57 | TS5 | 55 |
| 143 | EN1867 | 43 | EN1883 | 58 | TS5 | 56 |
| 144 | EN1867 | 43 | EN1884 | 59 | TS5 | 57 |
| 145 | EN1867 | 43 | EN1885 | 60 | TS5 | 58 |
| 146 | EN1868 | 44 | EN1871 | 46 | TS5 | 59 |
| 147 | EN1868 | 44 | EN1872 | 47 | TS5 | 60 |
| 148 | EN1868 | 44 | EN1873 | 48 | TS5 | 61 |
| 149 | EN1868 | 44 | EN1874 | 49 | TS5 | 62 |
| 150 | EN1868 | 44 | EN1875 | 50 | TS5 | 63 |
| 151 | EN1868 | 44 | EN1876 | 51 | TS5 | 64 |
| 152 | EN1868 | 44 | EN1877 | 52 | TS5 | 65 |
| 153 | EN1868 | 44 | EN1878 | 53 | TS5 | 66 |
| 154 | EN1868 | 44 | EN1879 | 54 | TS5 | 67 |
| 155 | EN1868 | 44 | EN1880 | 55 | TS5 | 68 |
| 156 | EN1868 | 44 | EN1881 | 56 | TS5 | 69 |
| 157 | EN1868 | 44 | EN1882 | 57 | TS5 | 70 |
| 158 | EN1868 | 44 | EN1883 | 58 | TS5 | 71 |
| 159 | EN1868 | 44 | EN1884 | 59 | TS5 | 72 |
| 160 | EN1868 | 44 | EN1885 | 60 | TS5 | 73 |
| 161 | EN1861 | 38 | EN1869 | 45 | TS6 | 26 |
| 162 | EN1862 | 39 | EN1869 | 45 | TS6 | 27 |
| 163 | EN1863 | 40 | EN1869 | 45 | TS6 | 28 |

### Example 4: Recombination at the Target Locus in Planta

To achieve recombination in planta, site-specific genome modification enzymes designed for specific target sequence(s) identified in the protoplast assay are cloned into transformation vectors containing expression cassettes with plant-specific expression elements and codon optimization for plant expression, as described in Example 2. The expression cassettes are incorporated into Agrobacterium transformation vectors. Plant transformation methods using Agrobacterium are known in the art. Alternatively, transformation vectors are introduced to the plant cells by biolistic transformation methods, which are also well known in the art. Following transformation, stable plants are selected using methods well known in the art.

Expression cassettes containing a tissue-specific promoter and/or a chemically inducible promoter, such as an alcohol inducible promoter, may be used to regulate expression of the site-specific genome modification enzyme. Where a chemically inducible promoter is used, expression of the site-specific genome modification enzyme can be induced at a desired growth stage after application of the chemical inducer. Optionally, expression cassettes with both a tissue-specific and chemically inducible promoter may be used so that expression of the site-specific genome modification enzyme occurs only in the desired tissue (for example, pollen) at the time and growth stage of application of the chemical inducer.

In planta targeting of selected genomic loci may be conducted such that the site-specific genome modification enzyme induces a genome modification, for example, a double strand break (DSB), a single-strand break, a transposase-mediated DNA exchange reaction or a recombinase-mediated DNA exchange reaction, within at least one target sequence within the genomic locus of interest. Introduction of a genome modification will increase the rate of recombination within the genomic locus of interest. Recombination events may be symmetric or unequal and may occur between conserved or divergent regions within the genomic locus of interest. Plant cells used for transformation are obtained from plants identified as described in Example 1. Further, the germplasm of the plant selected for transformation may be based, at least in part, on advantageous regeneration properties. Plant cells selected for transformation may be either homozygous or heterozygous at the genomic locus of interest. For example, corn plants comprising parental genomes which are non-identical at the Rp1 locus or soybean plants comprising parental genomes which are non-identical at the Rpp1 locus may be selected. Within a genomic locus of interest, whether the genomic locus of interest is homozygous or heterozygous, target sequences for site-specific genome modification enzymes may be selected to induce recombination between non-identical regions of the genomic locus of interest.

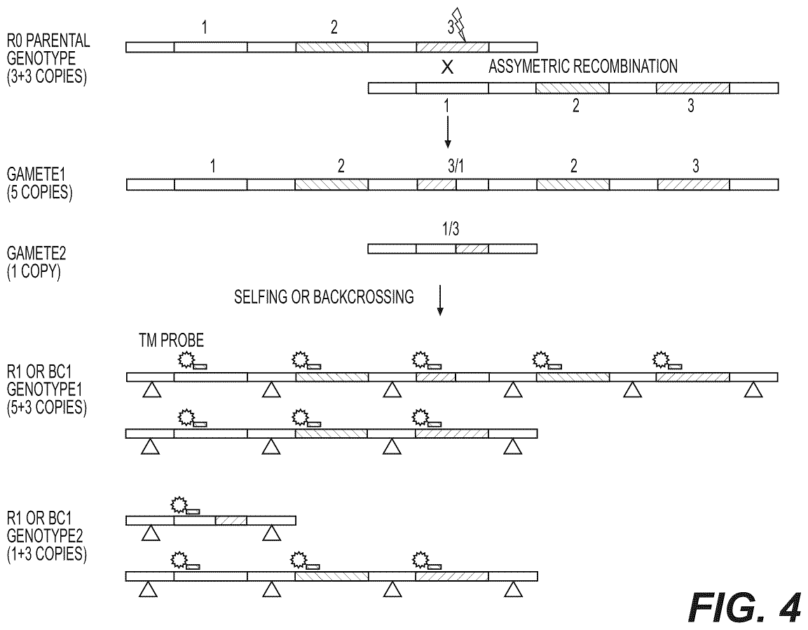

Following transformation and selection of stable plants, leaf samples are obtained for each plant, genomic DNA is extracted, and a high-throughput assay using the Droplet Digital™ PCR (ddPCR) technology is performed to identify individual plants with recombination at the selected genomic locus. The design of the PCR assay is illustrated in FIG. 4. Unequal recombination events are identified by detection of altered gene copy numbers in the genomic locus. In the example illustrated in FIG. 4, each parental genotype contributes three paralogs, so the cells selected for transformation have six paralogs (see top “R0 Parental” in FIG. 4). In events where recombination occurs, one possible example is illustrated where, following unequal recombination, one gamete has five paralogs in the genomic locus and one gamete has one paralog in the genomic locus (see “gamete 1” and “gamete 2” in FIG. 4). Following backcrossing, the genotype of the progeny from ‘gamete 1’ will have eight paralogs at the genomic locus; five paralogs on the allele from gamete 1 and three paralogs from the wild-type parent allele. Similarly, the genotype of the progeny from backcrossing ‘gamete 2’ will have four paralogs at the genomic locus; 1 paralog on the allele from gamete 1 and three paralogs from the wild-type parent allele. Because the PCR probe is designed to a common region in all paralogs within the locus, the ddPCR assay result reflects the number of paralogs. The variation in number paralogs from the parental paralog number is used to detect recombination events at the locus. For example, if the parental alleles each have three paralogs in the genomic locus (as illustrated in FIG. 4), then upon homologous asymmetric recombination, resulting gametes may have five paralogs, four paralogs, three paralogs, two paralogs, or one paralog at the locus. Upon either back-crossing or selfing, these gametes will result in plants with altered copy number, which is detected by the ddPCR technology.

### Example 5: Phenotypic Screening of Plants with Recombination to Identify Novel Disease Resistance Trait

The plants identified in Example 3 with molecularly confirmed recombination within the selected genomic locus are used for phenotypic screening to identify the plants with new or enhanced tolerance to biotic stress.

For early, high-throughput screening, a leaf disc assay is used. In these assays, leaf discs are collected and placed in a petri dish and inoculated with a pathogen using protocols known in the art. The dish is incubated at appropriate conditions to promote the pathogen growth and visual inspection is used to determine the resistance of the plant disc to damage by the pathogen.

Another high-throughput screening assay to identify plants with enhanced tolerance to a pathogen is the leaf inoculation assay wherein a leaf is removed from the selected plant and is placed in a petri dish, inoculated with the pathogen, and the dish incubated at appropriate conditions to promote pathogen growth. Visual inspection is used to determine the resistance of the leaf to damage by the pathogen.

Another high-throughput screening assay to identify plants with enhanced disease tolerance is a root elongation and hypocotyl elongation assay. In this assay, seed are plated either on agar or in rolled-filter paper and incubated in conditions to allow for root and hypotocytl elongation. The agar or filter paper are inoculated with the pathogen of interest. The length of the root and hypocotyl are measured as an indication of disease tolerance.

Another phenotypic screening assay to identify plants with enhanced pathogen tolerance is to grow individual plants in pots in a greenhouse, inoculation of the plant with a pathogen and monitoring phenotypic parameters of the plant growth. The plant growth parameters which are tracked are plant height, canopy development, root architecture, fresh weight, stalk strength, and any other plant health parameter. Collection of the plant growth parameter data is done manually or with the systems of an automated greenhouse.

Several imaging systems optimized for field or green house conditions are used for quick and accurate phenotyping of massive amounts of plants in a fraction of the time that manual methods require. Examples include 2D or 3D imaging of whole plants or selected organs (e.g. leaves) using a variety of technologies, such as fluorescence, thermal or infrared cameras in automated or traditional greenhouse, or field settings.

Plants having passed the high-throughput leaf disc assay or leaf inoculation assay, and green house assay, are then screened for biotic stress in field trials. For this, seeds from the plants with the identified recombinant alleles are planted in the field in complete block design, the plants may or may not be inoculated with a pathogen and plant growth parameters are scored and yield is determined. Plants with resistance to biotic stress are used in a breeding program to introgress the new allele into elite germplasm.

### Example 6. Accelerated Recombination in Rp1 Rust Resistance Locus of Corn

The Rp1 rust resistance locus of corn is one example of an NBS-LRR gene cluster of high agronomic value. In the development of new NBS-LRR alleles, recombination events can be either between matching copies of the NBS-LRR cluster or between mismatched copies of the NBS-LRR cluster. The B73 corn genome was re-sequenced in the Rp1 locus. Using the publicly available Rp1 gene models for annotation, altogether, 16 Rp1 paralogs were identified in the Rp1 locus on chromosome 10. Of the 16 Rp1 paralogs, 14 were clustered together, and the two other paralogs, while also located on chromosome 10, were separated by a larger chromosomal segment.

To identify target sequences useful for Streptococcus pyogenes Cas9 mediated double strand break (DSB) induced recombination in the Rp1 locus, the genomic loci encompassing each of the 16 identified Rp1 paralogs were analyzed using bioinfomatic tools. Two representative examples of Rp1 paralogs are presented as SEQ ID NO:1 and SEQ ID NO:2. The Rp1 paralogs were aligned using the Clustal W algorithm for multi-sequence alignment (Higgins et al. 1994). Regions of sequence which were conserved across most Rp1 paralogs were searched for both ‘optimal’ (G(N)₁₈GNGG; SEQ ID NO:78) and ‘minimal’ ((N₂₁)GG; SEQ ID NO:79) CRISPR/Cas9 target sequences (TS). The regions including such target sequences were further prioritized by their degree of conservation to maximize cutting efficiency: as a rule of thumb, at least half of all paralogs had to be cleavable by one, or at most two, homologous guide-RNA (gRNA) constructs. Among the regions that fulfilled all of the above criteria, the regions located in the C-terminal leucine-rich repeat (LRR) domain of the Rp1 paralogs were given preference for targeting to induce double strand breaks with the CRISPR/Cas9 nuclease. The LRR domains tend to be more divergent among paralogs than the NBS domains of NBS-LRR class of genes. Moreover, most determinants of resistance specificities are located in the LRR domain. For this reason, unequal recombination in the LRR domains is expected to accelerate generation of novel disease resistance alleles at a higher frequency than unequal recombination in the NBS domains.

From this analysis, 5 separate consensus sequences were identified. For target sequences (TS) 1 and 2, there were two variants of the consensus sequence that differed by 1 nucleotide. The consensus Rp1 target sequences are presented as SEQ ID NOs:3-9. For the two representative Rp1 paralogs (SEQ ID NO:1 and SEQ ID NO:2), the location of the different CRISPR/Cas9 target sequences is presented in Table 2.

TABLE 2
Position of Rp1 CRISPR/Cas9 target
sequences in representative Rp1 paralogs.

| | Rp1 | | | | |
|---|---|---|---|---|---|
| | Target | | TS SEQ | Start | End |
| Rp1 | Sequence | Target Sequence (TS) | ID NO | (bp) | (bp) |
| SEQ ID | TS-1a | GCATCTTCAAATTATTGAAAGTGG | 3 | 801 | 824 |
| NO: 2 | TS2-b | AATCTAGCACATATCCTGGGTGG | 6 | 389 | 411 |
| | TS-3 | CCTTCTTTAGAGCTAGCACGTGG | 7 | 1204 | 1226 |
| | TS-4 | GGCTCTTTTGCCATGAGCAGAGG | 8 | 878 | 900 |
| SEQ ID | TS-1b | GCATCTTCAAATCATTGAAAGTGG | 4 | 2766 | 2789 |
| NO: 1 | TS-2a | GATCTGATACATATCCTGGGTGG | 5 | 2351 | 2373 |
| | TS-3 | CCTTCTTTAGAGCTAGCACGTGG | 7 | 3169 | 3191 |
| | TS-4 | GGCTCTTTTGCCATGAGCAGAGG | 8 | 2843 | 2865 |
| | TS-5 | GCTTTAGCTATTTGCCTTGGTGG | 9 | 2962 | 2984 |

Across the 16 separate Rp1 paralogs evaluated for the presence of conserved CRISPR/Cas9 target sequences, 12 Rp1 paralogs had a CRISPR/Cas9 target sequence for TS-1 (TS-1a: SEQ ID NO:3 or TS-1b: SEQ ID NO:4); 10 of the 16 Rp1 paralogs had a CRISPR/Cas9 target sequence for TS-2 (TS-2a: SEQ ID NO:5, TS-2b: SEQ ID NO:6); 8 of the 16 Rp1 paralogs had a CRISPR/Cas9 target sequence for TS-3 (SEQ ID NO:7); 10 of the 16 Rp1 paralogs had a CRISPR/Cas9 target sequence for TS-4 (SEQ ID NO:8), and 8 of the 16 Rp1 paralogs had a CRISPR/Cas9 target sequence for TS-5 (SEQ ID NO:9).

Plant transformation vectors were designed to deliver CRISPR/Cas9 nuclease components. The Cas9 sequence is derived from Streptococcus pyogenes and the nucleotide sequence was codon optimized for monocot expression. Further, the Cas9 sequence contains a nuclear targeting sequence at both the 5′ and 3′ ends of the protein (SEQ ID NO:77). In the same transformation vector, there are one or two guide-RNA (gRNA) encoding cassettes: two gRNA cassettes for Rp1 TS-1 (TS-1a and TS-1b); two gRNA cassettes for Rp1 TS-2 (TS-2a and TS-2b); and one gRNA cassette for each vector designed to target Rp1 TS-3, or TS-4, or TS-5. The transformation vectors also contain a selection cassette conferring tolerance to glyposate. The transformation vectors are introduced into plants as described in Example 4.

DNA extraction methods for corn tissue are known in the art. Methods to identify the Rp1 locus, are known in the art by use of genomic markers. Methods of deep sequencing are known in the art and indicates that a region of DNA is sequenced multiple times (hundreds to thousands of times) to detect single nucleotide polymorphism (SNPs) with high accuracy. Non-limiting examples of methods to perform sequencing include Illumina® sequencing platform (Illumina, San Diego, CA), Roche 454 sequencing system (Roche, Branford, CT), single molecule, real-time (SMRT®) sequencing technology (Pacific Biosciences, Menlo Park, CA) sequencing platforms, and others known in the art.

In addition to the molecular screening of R0 and R1 plants, R1 or additional inbred or hybrid progeny plants are screened for disease resistance as described in Example 5.

### Example 7: Accelerated Recombination in Rpp1 Soybean Rust Resistance Locus

The Rpp1 resistance locus of soybean is one example of an NBS-LRR gene cluster of high agronomic value. In the development of new NBS-LRR alleles, recombination events can be either “equal” for example, between corresponding regions of the NBS-LRR cluster, or “unequal” for example, between non-corresponding regions of the NBS-LRR cluster. The genome of soybean from different germplasms was sequenced in the Rpp1 locus. Using the publicly available Rpp1 gene models for annotation, altogether, three Rpp1 paralogs were identified in the Rpp1 locus.

To identify target sites useful for Streptococcus pyogenes Cas9 mediated double strand break (DSB) induced recombination in the Rpp1 locus, the genomic regions encompassing each of the identified Rpp1 paralogs were analyzed using bioinfomatic tools. Two representative examples of Rpp1 paralogs identified in Williams 82 germplasm (W82) are presented as SEQ ID NO:10 and SEQ ID NO:11. The Rpp1 paralogs were aligned using the Clustal W algorithm for multi-sequence alignment (Higgins et al. 1994). Regions of sequence which were conserved across most Rpp1 paralogs were searched for both ‘optimal’ (G(N)₁₈GNGG; SEQ ID NO:57) and ‘minimal’ ((N₂₁)GG; SEQ ID NO:58) CRISPR/Cas9 target sequences (TS). The regions including such target sequences were further prioritized by the degree of their conservation to maximize cutting efficiency as described in Example 6.

From this analysis, 5 separate consensus sequences were identified. For target sequences (TS) 2 and 5, there were two variants of the consensus sequence that differed by 1 nucleotide. The consensus Rpp1 target sequences are presented as SEQ ID NOs: 12-18. For the two representative Rpp1 paralogs (SEQ ID NO:10 and SEQ ID NO:11), the location of the different CRISPR/Cas9 target sequences is presented in Table 3. Note that the target sequence (TS) position for TS-4 (SEQ ID NO:16, TS-5a (SEQ ID NO:17, and TS-5b (SEQ ID NO:18) are on the reverse strand as indicated by the start and end position of the respective SEQ ID NO:10 and SEQ ID NO:11.

TABLE 3
Position of Rpp1 CRISPR/Cas9 target
sequences in representative Rpp1 paralogs.

| | Rpp1 | | | | |
|---|---|---|---|---|---|
| | Target | | TS SEQ | Start | End |
| Rpp1 | Sequences | Target Sequence (TS) | ID NO | (bp) | (bp) |
| SEQ ID | TS-1 | GTGGGATCTTCTGGAGGATGAGG | 12 | 837 | 859 |
| NO: 10 | TS-2a | GTGGGTTGTTAAATGGAAAGGGG | 13 | 1661 | 1683 |
| | TS-3 | GGAATGGACAGCTGATCTGGAGG | 15 | 1872 | 1894 |
| | TS-4 | GCTTGTAGATCTCCCAGTGGAGG | 16 | 2171 | 2149 |
| | TS-5a | AAATAGATAAAATAGGTTTGAGG | 17 | 2489 | 2467 |
| SEQ ID | TS-1 | GTGGGATCTTCTGGAGGATGAGG | 12 | 1317 | 1339 |
| NO: 11 | TS2-b | GTGGGTTGTTAGATGGAAAGAGG | 14 | 2102 | 2124 |
| | TS-3 | GGAATGGACAGCTGATCTGGAGG | 15 | 2313 | 2335 |
| | TS-4 | GCTTGTAGATCTCCCAGTGGAGG | 16 | 2612 | 2590 |
| | TS-5b | AAATAGATAAAATAGATTTGAGG | 18 | 2930 | 2908 |

Across the 3 separate Rpp1 paralogs evaluated for the presence of conserved CRISPR/Cas9 target sequences, all 3 Rpp1 paralogs had a CRISPR/Cas9 target sequence for TS-1 (TS-1: SEQ ID NO:12), TS-3 (SEQ ID NO:15) and TS-4 (SEQ ID NO:16). One paralog had a CRISPR/Cas9 target sequence for both TS-2a (SEQ ID NO:13) and TS-2b (SEQ ID NO:14); two of the Rpp1 paralogs had a CRISPR/Cas9 target sequence for TS-5a (SEQ ID NO:17), and one of the paralogs a CRISPR/Cas9 target sequence for TS-5b (SEQ ID NO:18).

Plant transformation vectors were constructed to deliver CRISPR/Cas9 nuclease components. The Cas9 sequence is derived from Streptococcus pyogenes and the nucleotide sequence was codon optimized for monocot expression, and 13 amino acid changes were made to reduce allergenicity (E24D, D54G, V143A, T191I, G205R, K234R, T310S, T593A, E630G, H723Q, V743I, R753, L847I). Further, the Cas9 sequence contains a nuclear targeting sequence at both the 5′ and 3′ ends of the protein (SEQ ID NO:77). In the same transformation vector, there are one or two guide-RNA (gRNA) encoding cassettes: two gRNA cassettes for Rpp1 TS-2 (TS-2a and TS-2b); two gRNA cassettes for Rpp1 TS-5 (TS-5a and TS-5b); and one gRNA cassette for each vector designed to target Rpp1 TS-1, or TS-3, or TS-4. The transformation vectors also contain a selection cassette conferring tolerance to glyposate.

Plants identified with a genotype with polymorphisms suitable for recombination are crossed to create hybrid populations. The inbred parental germplasm or the F1 hybrid plants, or progeny heterozygous for the entire region of interest, are used as starting material for plant transformation. The plant transformation vectors are introduced into corn cells using methods known in the art, for example with Agrobacterium transformation or biolistic transformation. The transformed callus is selected with glyphosate using methods known in the art. The transformation vectors are introduced into plants as described in Example 4.

DNA extraction methods for corn tissue are known in the art. Methods to identify the Rp1 locus, are known in the art by use of genomic markers. Methods of deep sequencing are known in the art and indicates that a region of DNA is sequenced multiple times (hundreds to thousands of times) to detect single nucleotide polymorphism (SNPs) with high accuracy. Non-limiting examples of methods to perform sequencing include Illumina® sequencing platform (Illumina, San Diego, CA), Roche 454 sequencing system (Roche, Branford, CT), single molecule, real-time (SMRT®) sequencing technology (Pacific Biosciences, Menlo Park, CA) sequencing platforms, and others known in the art.

In addition to the molecular screening of R0 and R1 plants, R1 or additional inbred or hybrid progeny plants are screened for disease resistance as described in Example 5.

### Example 8: Accelerated Recombination in Rps1 Soybean Pytophthora Resistance Locus

The Rps1 Pytophthora resistance locus of soybean is one example of an NBS-LRR gene cluster of high agronomic value. In the development of new NBS-LRR genotypes, recombination events can be either “equal” for example, between corresponding regions of the NBS-LRR cluster, or “unequal” for example, between non-corresponding regions of the NBS-LRR cluster. The genome of soybean from different germplasms was sequenced in the Rps1 locus. Using the publicly available Rps1 gene models for annotation, altogether, 23 Rps1 paralogs were identified in the Rps1 soybean Pytophthora resistance locus.

From this analysis, 6 separate consensus sequences were identified, each with two variants of the consensus sequence that differed by 1 nucleotide. The consensus Rps1 target sequences are presented as SEQ ID NOs: 21-32. For the two representative Rps1 paralogs (SEQ ID NO:19 and SEQ ID NO:20), the location of the different CRISPR/Cas9 target sequences are presented in Table 4. Note that the target sequence (TS) position for TS-6a (SEQ ID NO:31) and TS-6b (SEQ ID NO:32) are on the reverse strand as indicated by the start and end position of the respective SEQ ID NO:19 and SEQ ID NO:20.

TABLE 4
Position of Rps1 CRISPR/Cas9 target
sites in representative Rps1 paralogs.

| | Rps1 | | | | |
|---|---|---|---|---|---|
| | Target | | TS SEQ | Start | End |
| Rps1 | Sequence | Target Sequence (TS) | ID NO | (bp) | (bp) |
| SEQ ID | TS-1a | GATCTAGCCACATCACTCGGTGG | 21 | 916 | 938 |
| NO: 19 | TS-2a | CCAAAGTGATGAAGCGTTGGAGG | 23 | 1557 | 1579 |
| | TS-3a | GGAATATCTTTTGGTTTCAGGGG | 25 | 2526 | 2548 |
| | TS-4a | ATTGAGTCCTTTCCAAAACGGGG | 27 | 2689 | 2711 |
| | TS-5a | TGGAGATGTTGGACTGCACAGGG | 29 | 2903 | 2925 |
| | TS-6a | GCAAACTTCCCTCTAGTTTGGGG | 31 | 2068 | 2046 |
| SEQ ID | TS-1b | GATCTAGCATTATACCTTGGTGG | 22 | 1375 | 1397 |
| NO: 20 | TS-2b | CAGAAGCAATGAAGCATTGGAGG | 24 | 1926 | 1948 |
| | TS-3b | GGAATCTCTTTTGGTTTCAGGGG | 26 | 2886 | 2908 |
| | TS-4b | ATTGAGTCGTTTCCAGAAGGGGG | 28 | 2953 | 2975 |
| | TS-5b | CTGGAGATGTTGGACTGCACGGGG | 30 | 3166 | 3189 |
| | TS-6b | GCAAATCTCCCCTTAGTTTGGGG | 32 | 2428 | 2406 |

Across the 23 Rps1 paralogs with sequence available, 21 were evaluated for the presence of conserved CRISPR/Cas9 target sequences and 7 Rps1 paralogs had a CRISPR/Cas9 target sequence for TS-1a (SEQ ID NO:21); 8 Rps1 paralogs had a CRISPR/Cas9 target sequence for TS-2a (SEQ ID NO:23); 6 Rps1 paralogs had a CRISPR/Cas9 target sequence for TS-3a (SEQ ID NO:25); 7 Rps1 paralogs had a CRISPR/Cas9 target sequence for TS-4a (SEQ ID NO:27); 1 Rps1 paralogs had a CRISPR/Cas9 target sequence for TS-5a (SEQ ID NO:29); 10 Rps1 paralogs had a CRISPR/Cas9 target sequence for TS-6a (SEQ ID NO:31); 5 Rps1 paralogs had a CRISPR/Cas9 target sequence for TS-1b (SEQ ID NO:22); 4 Rps1 paralogs had a CRISPR/Cas9 target sequence for TS-2b (SEQ ID NO:24); 7 Rps1 paralogs had a CRISPR/Cas9 target sequence for TS-3b (SEQ ID NO:26); 8 Rps1 paralogs had a CRISPR/Cas9 target sequence for TS-4b (SEQ ID NO:28); 12 Rps1 paralogs had a CRISPR/Cas9 target sequence for TS-5b (SEQ ID NO:30); and 4 Rps1 paralogs had a CRISPR/Cas9 target sequence for TS-6b (SEQ ID NO:32).

Plant transformation vectors were constructed to deliver CRISPR/Cas9 nuclease components as described in Example 6.

Plants identified with a genotype with polymorphisms suitable for recombination are transformed as described in Example 6.

DNA extraction methods and methods of identifying the Rp1 locus by use of genomic markers are known in the art. Deep sequencing is used to identify recombination events in the Rp1 locus.

In addition to the molecular screening of R0 and R1 plants, R1 or additional inbred or hybrid progeny plants are screened for disease resistance as described in Example 5.

### Example 9: Accelerated Recombination in Rhg1 Soybean Cyst Nematode Locus

The Rhg1 locus mediates nematode resistance in soybean and is one example of a disease resistance gene cluster locus of high agronomic value. The Rhg1 locus has been shown to contain three separate genes, and that susceptible soybean varieties contain only one copy of the Rhg1 locus. In contrast, at least one soybean germplasm line with resistance against soybean cyst nematode has an array of ten tandemly duplicated copies of the Rhg1 locus.

To identify regions of the Rhg1 locus to target for accelerated recombination, the publicly available genomic sequence for the Rhg1 locus in the Williams 82 germplasm (SEQ ID NO:68) was used to identify target sequences for a double-strand break inducing CRISPR/Cas9 nuclease. From this analysis eight target sequences (TS) were identified, TS1 (SEQ ID NO: 69); TS2 (SEQ ID NO: 70); TS3 (SEQ ID NO: 71); TS4 (SEQ ID NO: 72); TS5 (SEQ ID NO: 73); TS6 (SEQ ID NO: 74); TS7 (SEQ ID NO: 75); and TS8 (SEQ ID NO: 76). Six of the CRISPR/Cas9 target sequences are located within a genic region of the Rgh1 locus, and two of the CRISPR/Cas9 target sequences are located in an intergenic region of the Rhg1 locus (see Table 5).

TABLE 5
Rhg1 target sites and
their coordinates in target sequences

| | | | | | Genic (G) |
|---|---|---|---|---|---|
| SEQ | Target | | Start | End | Intergenic |
| ID NO | Sequence | Sequence | (bp) | (bp) | (I) |
| 69 | Rhg1_TS1 | ggcaaggcacactgcggatgagg | 4685 | 4707 | G |
| 70 | Rhg1_TS2 | gtacgctggcgtcatgagggagg | 6546 | 6524 | G |
| 71 | Rhg1_TS3 | ggcggccggagacatgccggagg | 6783 | 6805 | G |
| 72 | Rhg1_TS4 | ggaaattgctgaattgtacgagg | 10130 | 10152 | G |
| 73 | Rhg1_TS5 | gatcttaggctctttgaacgagg | 15140 | 15118 | G |
| 74 | Rhg1_TS6 | gggaagcttgcgatcgggtgcgg | 20233 | 20211 | I |
| 75 | Rhg1_TS7 | gaatcggaaggagttgtcggcgg | 20597 | 20575 | G |
| 76 | Rhg1_TS8 | ggctgattctaccgcgaccgtgg | 22792 | 22770 | I |

Plant transformation vectors are constructed to deliver CRISPR/Cas9 nuclease components as described in Example 1.

Plants identified with a genotype with polymorphisms suitable for recombination are transformed using methods known in the art and as described in Example 6.

DNA extraction methods and methods for identifying the Rhg1 locus are known in the art. Deep sequencing is used to identify modifications within the Rhg1 locus.

In addition to the molecular screening of R0 and R1 plants, R1 or additional inbred or hybrid progeny plants are screened for disease resistance as described in Example 5.

What is claimed is:

1. A method of generating a new gene array, comprising contacting a cell with a first site-specific genome modification enzyme that introduces a genome modification in at least one target sequence in a first gene array, thereby inducing recombination with a second gene array, and selecting at least one progeny comprising a new array of genes.

2. The method of claim 1, wherein the first site-specific genome modification enzyme introduces a genome modification in at least one target sequence in the second gene array.

3. The method of claim 1 or 2, further comprising contacting the cell with a second site-specific genome modification enzyme that introduces a genome modification in at least one target sequence in the second gene array.

4. The method of any one of claims 1-3, wherein the first and second gene arrays are arrays of tandemly duplicated genes; or wherein the first and second gene arrays are multigene families.

5. The method of any one of claims 1-4, wherein genes within the first and second gene arrays are paralogs.

6. The method of any one of claims 1-5, wherein the first and second gene arrays are homologous; wherein the first and second gene arrays are heterologous; wherein the first and second gene arrays are homoeologous; wherein the first and second gene arrays are paraologous; wherein the first and second gene arrays are identical; or wherein the first and second gene arrays are not identical.

7. The method of any one of claims 1-6, wherein the genome modification is a double strand break (DSB), a single strand break, a transposase-mediated DNA exchange reaction or a recombinase-mediated DNA exchange reaction.

8. The method of any one of claims 1-7, wherein the recombination between the first gene array and the second gene array is asymmetric.

9. The method of claim 1, 2, or 3, wherein the least one target sequence is within a gene.

10. The method of claim 1, 2, or 3, wherein the least one target sequence is within an intergenic region.

11. The method of claim 2, or 3, wherein the at least one target sequence in the first gene array is within an intergenic region and the at least one target sequence in the second gene array is within a genic region; wherein the at least one target sequence in the first gene array is within a genic region and the at least one target sequence in the second gene array is within an intergenic region; wherein the at least one target sequence in the first gene array is within an intergenic region and the at least one target sequence in the second gene array is within an intergenic region; wherein the at least one target sequence in the first gene array is within a genic region and the at least one target sequence in the second gene array is within a genic region; wherein the at least one target sequence in the first gene array and the at least one target sequence in the second gene array are the same; wherein the at least one target sequence in the first gene array and the at least one target sequence in the second gene array are different; or wherein the at least one target sequence in the first gene array has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the at least one target sequence of the second gene array.

12. The method of any of claims 1-11, wherein a genomic locus comprising the target sequence for the site-specific genome modification enzyme in the first gene array is homologous to at least about 100 bp, at least about 150 bp, at least about 200 bp, at least about 250 bp, at least about 300 bp, at least about 350 bp, at least about 400 bp, at least about 450 bp, at least about 500 bp, at least about 600 bp, at least about 700 bp, at least about 800 bp, at least about 900 bp, or at least about 1000 bp of the second gene array.

13. The method of claim 12, wherein the regions of homology are in corresponding positions in the first and second gene arrays; or wherein the regions of homology are in different positions in the first and second gene arrays.

14. The method of any of claims 1-13, wherein the new gene array has an increased number of genes compared to the first gene array or the second gene array; or wherein the new gene array has a reduced number genes compared to the first gene array or the second gene array.

15. The method of any of claims 1-14, wherein said cell is a plant cell.

16. The method of claim 15, wherein said plant cell is obtained from an inbred or a hybrid plant.

17. The method of any one of claims 1-16, wherein the new gene array, the first gene array and the second gene array encode proteins selected from: NBS-LRR disease resistance proteins, pathogen recognition receptor (PRR) proteins, seed storage proteins, cell wall component extension proteins, F-box proteins, ABC transporters, and serine-threonine/tyrosine protein kinases.

18. The method of any one of claims 1-17, wherein the new gene array encodes one or more proteins that confer resistance to at least one disease selected from the group consisting of Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis).

19. The method of claim 18, wherein the new gene array provides improved resistance to at least one disease compared to the first gene array or the second gene array.

20. A method of providing a plant with improved disease resistance, comprising:
a. providing to one or more plant cells a site-specific genome modification enzyme that introduces a genome modification at least one target sequence in a disease resistance locus;
b. screening for asymmetric recombination between disease-resistance loci on homologous chromosomes to identify plant cells comprising a recombinant disease resistance locus;
c. testing plants obtained from the plant cells identified in step (b) and their progeny for improved disease resistance; and
d. selecting the plant with improved disease resistance.

21. A method of generating a plant from an inbred line with an altered disease resistance locus compared to a disease resistance locus in a parental genome, comprising providing a site-specific genome modification enzyme to a plant cell, wherein the site-specific genome modification enzyme introduces a genome modification at least one target sequence in one or more disease resistance loci thereby inducing asymmetric recombination between the disease resistance locus in the first parental genome and the disease resistance locus in the second parental genome and growing the plant with the altered disease resistance locus from the plant cell.

22. The method of claim 20 or 21, wherein the site-specific genome modification enzyme introduces a genome modification in a second target sequence in the disease resistance locus.

23. The method of claim 20 or 21, further comprising contacting the plant cell with a second site-specific genome modification enzyme that introduces a genome modification at a different target sequence in the disease resistance locus.

24. The method of any one of claims 20-23, wherein the site-specific genome modification enzyme induces one or more of: a double strand break (DSB), a single strand break, a transposase-mediated DNA exchange reaction and a recombinase-mediated DNA exchange reaction.

25. The method of claim 24, wherein the site-specific genome modification enzyme introduces a double-strand break (DSB) at least twice in the disease resistance loci thereby resulting in a deletion of a sequence in the disease resistance locus.

26. The plant of claim 21, wherein the plant has improved disease resistance compared a plant of the inbred line without the altered disease resistance locus.

27. The method of claim 20 or 21, wherein the disease resistance locus encodes one or more nucleotide-binding site leucine-rich repeat (NBS-LRR) disease resistance proteins.

28. The method of claim 20 or 21, wherein the plant is corn and the disease resistance locus is Rp1; wherein said plant is soy and the disease resistance locus is Rpp1; wherein said plant is soy and the disease resistance locus is Rps1; or wherein the plant is soy and the disease resistance locus is Rhg1.

29. The method of claim 20 or 21, wherein said disease resistance locus confers resistance to one or more diseases selected from Anthracnose Stalk Rot (Colletotrichum graminicola), Fusarium Ear Rot (Fusarium verticillioides), Fusarium Stalk Rot (Fusarium spp.), Gibberella Ear Rot (Gibberella moniliformis), Gibberella Stalk Rot (Gibberella zeae), Goss's Wilt and Leaf Blight (Clavibacter michiganensis), Gray Leaf Spot (Cercospora zeae-maydis, C. zeina), Northern Corn Leaf Blight (Exserohilum turcicum), Sudden death syndrome (Fusarium solani f.sp. glycines), Asian soybean rust (Phakopsora pachyrhizi), Phytophthora root and stem rot (Phytophthora sojae), Root-knot Nematode (Meloidogyne spp.), Soybean Cyst Nematode (Heterodera glycines), Reniform nematode (Rotylenchulus reniformis), Root-knot nematode (Meloidogyne incognita), Fusarium wilt (Fusarium oxysporurn f. sp. vasinfectum), Verticillium wilt (Verticillium dahlia), Fusarium head blight (Fusarium graminearum), Fusarium seedling blight (Fusarium spp., Septoria nodorum), Fusarium Leaf Blotch (Monographella nivalis), and Stem Rust (Puccinia graminis).

30. The method of claim 20 or 21, wherein the disease resistance locus comprises one or more selected independently from the group consisting of a gene, an array of tandemly duplicated genes, a family of genes, an enhancer, a suppressor, a promoter, a termination sequence, a splice acceptor sequence, a splice donor sequence, an intron, an exon, an siRNA, and a quantitative trait locus (QTL).

31. The method of claim 21, wherein one or more of the first parental genome and the second parental genome are haploid.

32. The method of claim 21, wherein one or more of the first parental genome and the second parental genome are diploid.

33. The method of any one of claims 1-32, wherein the site-specific genome modification enzyme is selected from an endonuclease, a recombinase, a transposase, a helicase or any combination thereof.

34. The method of claim 33, wherein the endonuclease is selected from a meganuclease, a zinc finger nuclease, a transcription activator-like effector nuclease (TALEN), an Argonaute, a DNA-guided recombinase, a DNA-guided endonuclease, an RNA-guided recombinase, an RNA-guided endonuclease, a type I CRISPR-Cas system, type II CRISPR-Cas system and a type III CRISPR-Cas system.

35. The method of claim 33, wherein the endonuclease is selected from the group comprising Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease.

36. The method of any one of claims 1-32, wherein the site-specific genome modification enzyme is dCas9-recombinase fusion protein.

37. The method of claim 33 or 36, wherein the recombinase is a tyrosine recombinase attached to a DNA recognition motif, or a serine recombinase attached to a DNA recognition motif.

38. The method of claim 37, wherein the tyrosine recombinase attached to a DNA recognition motif is selected from the group consisting of a Cre recombinase, a Flp recombinase, and a Tnp1 recombinase.

39. The method of claim 37 wherein the serine recombinase attached to a DNA recognition motif is selected from the group consisting of a PhiC31 integrase, an R4 integrase, and a TP-901 integrase.

40. The method of claim 33, wherein said transposase is a DNA transposase attached to a DNA binding domain.

41. A plant, plant cell or a seed of a plant produced by the method of any one of claims 1-41.
